(19) United States
(12) Patent Application Publication
(10) Pub. No.: US 2011/0070232 A1
(43) Pub. Date: Mar. 24, 2011

(54) Combination Therapy with an Antitumor Alkaloid

(75) Inventors: Doreen LePage, Cambridge, MA (US); Pablo Manuel Aviles Marin, Madrid (ES); Maria Jose Guillen Navarro, Madrid (ES)

(73) Assignee: Pharma Mar, S.A., Madrid (ES)

(21) Appl. No.: 12/992,812

(22) PCT Filed: May 18, 2009
(86) PCT No.: PCT/US09/44334

§ 371 Date: Nov. 15, 2010

Related U.S. Application Data
(60) Provisional application No. 61/053,726, filed on May 16, 2008.

(51) Int. Cl. A61K 31/4995; A61K 31/519; A61K 31/7068; A61K 39/395; A61K 33/24; A61K 31/517; A61K 31/505; A61K 31/704; A61P 35/00

(52) U.S. Cl. 424/133.1; 514/250; 514/49; 424/649; 514/34

(57) ABSTRACT

The present invention relates to combinations of PM00104 with other anticancer drugs, and the use of these combinations in the treatment of cancer.

### FIELD OF THE INVENTION

The present invention relates to the combination of PM00104 with other anticancer drugs, in particular other anticancer drugs selected from antitumor platinum coordination complexes, antimetabolites, mitotic inhibitors, anthracyclines, topoisomerase I and/or II inhibitors, antitumor monoclonal antibodies, mTOR inhibitors, and tyrosine kinase inhibitors.

### BACKGROUND OF THE INVENTION

Cancer develops when cells in a part of the body begin to grow out of control. Although there are many kinds of cancer, they all arise from out-of-control growth of abnormal cells. Cancer cells can invade nearby tissues and can spread through the bloodstream and lymphatic system to other parts of the body. There are several main types of cancer. Carcinoma is a malignant neoplasm, which is an uncontrolled and progressive abnormal growth, arising from epithelial cells. Epithelial cells cover internal and external surfaces of the body, including organs, lining of vessels and other small cavities. Sarcoma is cancer arising from cells in bone, cartilage, fat, muscle, blood vessels, or other connective or supportive tissue. Leukemia is cancer that arises in blood-forming tissue such as the bone marrow, and causes large numbers of abnormal blood cells to be produced and enter the bloodstream. Lymphoma and multiple myeloma are cancers that arise from cells of the immune system.

In addition, cancer is invasive and tends to infiltrate the surrounding tissues and give rise to metastases. It can spread directly into surrounding tissues and also may be spread through the lymphatic and circulatory systems to other parts of the body.

Many treatments are available for cancer, including surgery and radiation for localised disease, and chemotherapy. However, the efficacy of available treatments for many cancer types is limited, and new, improved forms of treatment showing clinical benefits are needed. This is especially true for those patients presenting with advanced and/or metastatic disease and for patients relapsing with progressive disease after having been previously treated with established therapies which become ineffective or intolerable due to acquisition of resistance or to limitations in administration of the therapies due to associated toxicities.

Since the 1950s, significant advances have been made in the chemotherapeutic management of cancer. Unfortunately, more than 50% of all cancer patients either do not respond to initial therapy or experience relapse after an initial response to treatment and ultimately die from progressive metastatic disease. Thus, the ongoing commitment to the design and discovery of new anticancer agents is critically important.

The ideal antitumor drug would kill cancer cells selectively, with a wide index relative to its toxicity towards non-cancer cells, and would also retain its efficacy against cancer cells, even after prolonged exposure to the drug. Unfortunately, none of the current chemotherapies with known agents posses an ideal profile. Most posses very narrow therapeutic indexes and, in addition, cancerous cells exposed to slightly sublethal concentrations of a chemotherapeutic agent may develop resistance to such an agent, and quite often cross-resistance to several other antitumor agents.

PM00104 is an alkaloid related to jorumycin and renieramycins, and also to safracin and saframycin compounds. Jorumycin is a natural compound isolated from the skin and from the mucus of the Pacific nudibranch Jorunna funebris (Fontana A., et al., Tetrahedron (2000), 56, 7305-8). In addition, the family of renieramycins is disclosed as being isolated from sponges and tunicates (James M. F. et al. J. Am. Chem. Soc. (1982), 104, 265-269; Oku N., et al. Journal Natural Products (2003), 66, 1136-9). Safracin and saframycin compounds are disclosed in Manzanares I., et al. Curr. Med. Chem. Anti-Cancer Agents (2001), 1, 257-276, as well as in WO 00/18233 and WO 01/87894.

PM00104 has demonstrated significant in vitro activity against solid and non-solid tumour cell lines as well as significant in vivo activity in several xenografted human cell lines in mice, such as breast and prostate. Preliminary insights into the mechanism of action of PM00104 suggested cell cycle changes, DNA binding properties and transcriptional inhibition. This compound has the following chemical structure:

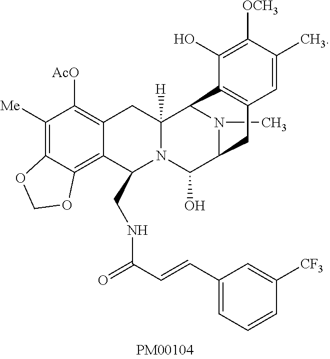

For further details of PM00104 see WO 01/87894. Additionally, the reader is referred to WO 2007/052076 and WO 2008/135792 which are incorporated herein by specific reference, for pharmaceutical compositions and administration dosages and schedules of PM00104.

Since cancer is a leading cause of death in animals and humans, several efforts have been and are still being undertaken in order to obtain a safe and effective therapy to be administered to patients suffering from a cancer. The problem to be solved by the present invention is to provide anticancer therapies that are useful in the treatment of cancer.

### SUMMARY OF THE INVENTION

The present invention establishes that PM00104 potentiates the antitumor activity of other anticancer agents, in particular other anticancer drugs selected from antitumor platinum coordination complexes, antimetabolites, mitotic inhibitors, anthracyclines, topoisomerase I and/or II inhibitors, antitumor monoclonal antibodies, mTOR inhibitors, and tyrosine kinase inhibitors, and therefore PM00104 and other anticancer agents can be successfully used in combination therapy for the treatment of cancer.

Thus, this invention is directed to pharmaceutical compositions, kits, methods for the treatment of cancer using combination therapies, and uses of PM00104 in the manufacture of a medicament for combination therapy.

In accordance with one aspect of this invention, we provide effective combination therapies for the treatment of cancer based on PM00104, or a pharmaceutically acceptable salt thereof, and using another anticancer drug.

In another embodiment, the invention encompasses a method of treating cancer comprising administering to a patient in need of such treatment a therapeutically effective amount of PM00104, or a pharmaceutically acceptable salt thereof, and a therapeutically effective amount of another anticancer drug, administered prior, during, or after administering PM00104. The two drugs may form part of the same composition, or be provided as a separate composition for administration at the same time or at a different time.

In another aspect, the invention encompasses a method of increasing or potentiating the therapeutic efficacy of an anticancer drug in the treatment of cancer, which comprises administering to a patient in need thereof a therapeutically effective amount of PM00104, or a pharmaceutically acceptable salt thereof. PM00104 is administered prior, during, or after administering the other anticancer drug.

In another embodiment, the invention encompasses the use of PM00104, or a pharmaceutically acceptable salt thereof, for the manufacture of a medicament for the treatment of cancer, in combination therapy with another anticancer drug.

In a further aspect, the invention encompasses a pharmaceutical composition comprising PM00104, or a pharmaceutically acceptable salt thereof, and/or another anticancer drug, and a pharmaceutically acceptable carrier or excipient, to be used in combination therapy for the treatment of cancer.

The invention also encompasses a kit for use in the treatment of cancer which comprises a dosage form of PM00104, or a pharmaceutically acceptable salt thereof, and/or a dosage form of another anticancer drug, and instructions for the use of both drugs in combination.

In one preferred aspect, the present invention is concerned with synergistic combinations of PM00104, or a pharmaceutically acceptable salt thereof, with another anticancer drug.

### BRIEF DESCRIPTION OF THE FIGURES

FIG. 1-3. Inhibitory effects of PM00104 and cisplatin combinations in Hs746T (FIG. 1), AGS (FIG. 2), and HGC-27 (FIG. 3) cells.

FIG. 4-6. Inhibitory effects of PM00104 and SN38 combinations in Hs746T (FIG. 4), AGS (FIG. 5), and HGC-27 (FIG. 6) cells.

FIG. 7-9. Inhibitory effects of PM00104 and 5-FU combinations in Hs746T (FIG. 7), AGS (FIG. 8), and HGC-27 (FIG. 9) cells.

FIG. 10-12. Inhibitory effects of PM00104 and doxorubicin combinations in Hs746T (FIG. 10), AGS (FIG. 11), and HGC-27 (FIG. 12) cells.

FIG. 13-15. Inhibitory effects of PM00104 and docetaxel combinations in Hs746T (FIG. 13), AGS (FIG. 14), and HGC-27 (FIG. 15) cells.

FIG. 16-18. Inhibitory effects of PM00104 and oxaliplatin combinations in Hs746T (FIG. 16), AGS (FIG. 17), and HGC-27 (FIG. 18) cells.

FIG. 19-20. Inhibitory effects of PM00104 and gemcitabine (Gemzar®) combinations in 5637 (FIG. 19) and UM-UC-3 (FIG. 20) cells.

FIG. 21-22. Inhibitory effects of PM00104 and cisplatin combinations in 5637 (FIG. 21) and UM-UC-3 (FIG. 22) cells.

FIG. 23-26. Inhibitory effects of PM00104 and gemcitabine (Gemzar®) combinations in BxPC-3 (FIG. 23), PANC-1 (FIG. 24), MIA PaCa-2 (FIG. 25), and SW1990 (FIG. 26) cells.

FIG. 27. Tumor volume evaluation (mean±SEM) of MIA PaCa-2 tumors in mice treated with control, PM00104 (0.9 mg/kg/day), gemcitabine (140 mg/kg/day) or PM00104 plus gemcitabine.

FIG. 28. Tumor volume evaluation (mean±SEM) of MIA PaCa-2 tumors in mice treated with control, PM00104 (0.9 mg/kg/day), erlotinib (100 mg/kg/day) or PM00104 plus erlotinib.

FIG. 29. Tumor volume evaluation (mean±SEM) of MIA PaCa-2 tumors in mice treated with control, PM00104 (0.9 mg/kg/day), erlotinib (50 mg/kg/day) or PM00104 plus erlotinib.

FIG. 30. Tumor volume evaluation (mean±SEM) of BxPC-3 tumors in mice treated with control, PM00104 (0.9 mg/kg/day), gemcitabine (180 mg/kg/day) or PM00104 plus gemcitabine.

FIG. 31. Tumor volume evaluation (mean±SEM) of BxPC-3 tumors in mice treated with control, PM00104 (0.9 mg/kg/day), erlotinib (50 mg/kg/day) or PM00104 plus erlotinib.

FIG. 32. Tumor volume evaluation (mean±SEM) of BxPC-3 tumors in mice treated with control, PM00104 (0.9 mg/kg/day), erlotinib (30 mg/kg/day) or PM00104 plus erlotinib.

FIG. 33. Tumor volume evaluation (mean±SEM) of BxPC-3 tumors in mice treated with control, PM00104 (0.9 mg/kg/day), erlotinib (15 mg/kg/day) or PM00104 plus erlotinib.

FIG. 34. Tumor volume evaluation (mean±SEM) of UM-UC-3 tumors in mice treated with control, PM00104 (0.9 mg/kg/day), cisplatin (5 mg/kg/day) or PM00104 plus cisplatin.

FIG. 35. Tumor volume evaluation (mean±SEM) of UM-UC-3 tumors in mice treated with control, PM00104 (0.9 mg/kg/day), gemcitabine (180 mg/kg/day) or PM00104 plus gemcitabine.

FIG. 36. Tumor volume evaluation (mean±SEM) of UM-UC-3 tumors in mice treated with control, PM00104 (0.9 mg/kg/day), paclitaxel (15 mg/kg/day) or PM00104 plus paclitaxel.

FIG. 37. Tumor volume evaluation (mean±SEM) of Hs746T tumors in mice treated with control, PM00104 (0.9 mg/kg/day), cisplatin (5 mg/kg/day) or PM00104 plus cisplatin.

FIG. 38. Tumor volume evaluation (mean±SEM) of Hs746T tumors in mice treated with control, PM00104 (0.9 mg/kg/day), paclitaxel (10 mg/kg/day) or PM00104 plus paclitaxel.

FIG. 39. Tumor volume evaluation (mean±SEM) of Hs746T tumors in mice treated with control, PM00104 (0.9 mg/kg/day), 5-FU (50/100 mg/kg/day) or PM00104 plus 5-FU.

FIG. 40. Tumor volume evaluation (mean±SEM) of Hs746T tumors in mice treated with control, PM00104 (0.9 mg/kg/day), irinotecan (20 mg/kg/day) or PM00104 plus irinotecan.

FIG. 41. Tumor volume evaluation (mean±SEM) of Hs746T tumors in mice treated with control, PM00104 (0.9 mg/kg/day), doxorubicin (6 mg/kg/day) or PM00104 plus doxorubicin.

FIG. 42. Tumor volume evaluation (mean±SEM) of Hs746T tumors in mice treated with control, PM00104 (0.9 mg/kg/day), docetaxel (16 mg/kg/day) or PM00104 plus docetaxel.

FIG. 43. Tumor volume evaluation (mean±SEM) of Hs746T tumors in mice treated with control, PM00104 (0.9 mg/kg/day), docetaxel (8 mg/kg/day) or PM00104 plus docetaxel.

FIG. 44. Tumor volume evaluation (mean±SEM) of Hs746T tumors in mice treated with control, PM00104 (0.9 mg/kg/day), oxaliplatin (8 mg/kg/day) or PM00104 plus oxaliplatin.

FIG. 45. Tumor volume evaluation (mean±SEM) of Hs746T tumors in mice treated with control, PM00104 (0.9 mg/kg/day), oxaliplatin (4 mg/kg/day) or PM00104 plus oxaliplatin.

FIG. 46. Tumor volume evaluation (mean±SEM) of MRI-H-254 tumors in mice treated with control, PM00104 (0.9 mg/kg/day), 5-FU (100 mg/kg/day) or PM00104 plus 5-FU.

FIG. 47. Tumor volume evaluation (mean±SEM) of MRI-H-254 tumors in mice treated with control, PM00104 (0.9 mg/kg/day), 5-FU (50 mg/kg/day) or PM00104 plus 5-FU.

FIG. 48. Tumor volume evaluation (mean±SEM) of MRI-H-254 tumors in mice treated with control, PM00104 (0.9 mg/kg/day), docetaxel (16 mg/kg/day) or PM00104 plus docetaxel.

FIG. 49. Tumor volume evaluation (mean±SEM) of MRI-H-254 tumors in mice treated with control, PM00104 (0.9 mg/kg/day), docetaxel (8 mg/kg/day) or PM00104 plus docetaxel.

FIG. 50. Tumor volume evaluation (mean±SEM) of MRI-H-254 tumors in mice treated with control, PM00104 (0.9 mg/kg/day), oxaliplatin (8 mg/kg/day) or PM00104 plus oxaliplatin.

FIG. 51. Tumor volume evaluation (mean±SEM) of MRI-H-254 tumors in mice treated with control, PM00104 (0.9 mg/kg/day), oxaliplatin (4 mg/kg/day) or PM00104 plus oxaliplatin.

FIG. 52. Tumor volume evaluation (mean±SEM) of MRI-H-254 tumors in mice treated with control, PM00104 (0.9 mg/kg/day), doxorubicin (6 mg/kg/day) or PM00104 plus doxorubicin.

FIG. 53. Tumor volume evaluation (mean±SEM) of MRI-H-254 tumors in mice treated with control, PM00104 (0.45 mg/kg/day), doxorubicin (6 mg/kg/day) or PM00104 plus doxorubicin.

FIG. 54. Tumor volume evaluation (mean±SEM) of MRI-H-254 tumors in mice treated with control, PM00104 (0.23 mg/kg/day), doxorubicin (6 mg/kg/day) or PM00104 plus doxorubicin.

FIG. 55. Tumor volume evaluation (mean±SEM) of MRI-H-254 tumors in mice treated with control, PM00104 (0.9 mg/kg/day), paclitaxel (12.5 mg/kg/day) or PM00104 plus paclitaxel.

FIG. 56. Tumor volume evaluation (mean±SEM) of MRI-H-254 tumors in mice treated with control, PM00104 (0.45 mg/kg/day), paclitaxel (12.5 mg/kg/day) or PM00104 plus paclitaxel.

FIG. 57. Tumor volume evaluation (mean±SEM) of MRI-H-254 tumors in mice treated with control, PM00104 (0.23 mg/kg/day), paclitaxel (12.5 mg/kg/day) or PM00104 plus paclitaxel.

FIG. 58. Tumor volume evaluation (mean±SEM) of MRI-H-254 tumors in mice treated with control, PM00104 (0.9 mg/kg/day), cisplatin (5 mg/kg/day) or PM00104 plus cisplatin.

FIG. 59. Tumor volume evaluation (mean±SEM) of MRI-H-254 tumors in mice treated with control, PM00104 (0.9 mg/kg/day), cisplatin (3 mg/kg/day) or PM00104 plus cisplatin.

FIG. 60. Tumor volume evaluation (mean±SEM) of MRI-H-254 tumors in mice treated with control, PM00104 (0.9 mg/kg/day), irinotecan (18 mg/kg/day) or PM00104 plus irinotecan.

FIG. 61. Tumor volume evaluation (mean±SEM) of MRI-H-254 tumors in mice treated with control, PM00104 (0.9 mg/kg/day), irinotecan (10 mg/kg/day) or PM00104 plus irinotecan.

FIG. 62. Tumor volume evaluation (mean±SEM) of MRI-H-254 tumors in mice treated with control, PM00104 (0.9 mg/kg/day), paclitaxel (25 mg/kg/day) or PM00104 plus paclitaxel.

FIG. 63. Tumor volume evaluation (mean±SEM) of MRI-H-254 tumors in mice treated with control, PM00104 (0.9 mg/kg/day), paclitaxel (12.5 mg/kg/day) or PM00104 plus paclitaxel.

FIG. 64. Tumor volume evaluation (mean±SEM) of HepG2 tumors in mice treated with control, PM00104 (0.9 mg/kg/day), sorafenib (60 mg/kg/day) or PM00104 plus sorafenib.

FIG. 65. Tumor volume evaluation (mean±SEM) of HepG2 tumors in mice treated with control, PM00104 (0.9 mg/kg/day), sorafenib (30 mg/kg/day) or PM00104 plus sorafenib.

FIG. 66. Tumor volume evaluation (mean±SEM) of HepG2 tumors in mice treated with control, PM00104 (0.6 mg/kg/day), sorafenib (60 mg/kg/day) or PM00104 plus sorafenib.

FIG. 67. Tumor volume evaluation (mean±SEM) of HepG2 tumors in mice treated with control, PM00104 (0.6 mg/kg/day), sorafenib (30 mg/kg/day) or PM00104 plus sorafenib.

FIG. 68. Tumor volume evaluation (mean±SEM) of PLC/PRF/5 tumors in mice treated with control, PM00104 (0.9 mg/kg/day), sorafenib (60 mg/kg/day) or PM00104 plus sorafenib.

FIG. 69. Tumor volume evaluation (mean±SEM) of PLC/PRF/5 tumors in mice treated with control, PM00104 (0.9 mg/kg/day), sorafenib (30 mg/kg/day) or PM00104 plus sorafenib.

FIG. 70. Tumor volume evaluation (mean±SEM) of PLC/PRF/5 tumors in mice treated with control, PM00104 (0.45 mg/kg/day), sorafenib (60 mg/kg/day) or PM00104 plus sorafenib.

FIG. 71. Tumor volume evaluation (mean±SEM) of PLC/PRF/5 tumors in mice treated with control, PM00104 (0.45 mg/kg/day), sorafenib (30 mg/kg/day) or PM00104 plus sorafenib.

FIG. 72. Tumor volume evaluation (mean±SEM) of SK-OV-3 tumors in mice treated with control, PM00104 (0.9 mg/kg/day), bevacizumab (5 mg/kg/day) or PM00104 plus bevacizumab.

FIG. 73. Tumor volume evaluation (mean±SEM) of SK-OV-3 tumors in mice treated with control, PM00104 (0.9 mg/kg/day), bevacizumab (2.5 mg/kg/day) or PM00104 plus bevacizumab.

FIG. 74-76. Inhibitory effects of PM00104 in combination with gemcitabine in lung cancer cell lines: A-549 (FIG. 74), NCI-H460 (FIG. 75), and NCI-H23 (FIG. 76) cells.

FIG. 77-79. Inhibitory effects of PM00104 in combination with paclitaxel in lung cancer cell lines: A-549 (FIG. 77), NCI-H460 (FIG. 78), and NCI-H23 (FIG. 79) cells.

FIG. 80-82. Inhibitory effects of PM00104 in combination with cisplatin in lung cancer cell lines: A-549 (FIG. 80), NCI-H460 (FIG. 81), and NCI-H23 (FIG. 82) cells.

FIG. 83-85. Inhibitory effects of PM00104 in combination with gemcitabine in breast cancer cell lines: MDA-MB-231 (FIG. 83), BT-474 (FIG. 84), and MCF-7 (FIG. 85) cells.

FIG. 86-88. Inhibitory effects of PM00104 in combination with paclitaxel in breast cancer cell lines: MDA-MB-231 (FIG. 86), BT-474 (FIG. 87), and MCF-7 (FIG. 88) cells.

FIG. 89-91. Inhibitory effects of PM00104 in combination with doxorubicin in breast cancer cell lines: MDA-MB-231 (FIG. 89), BT-474 (FIG. 90), and MCF-7 (FIG. 91) cells.

FIG. 92-94. Inhibitory effects of PM00104 in combination with 5-fluorouracil (5-FU) in colon cancer cell lines: HCT-116 (FIG. 92), LoVo (FIG. 93), and HT-29 (FIG. 94) cells.

FIG. 95-97. Inhibitory effects of PM00104 in combination with oxaliplatin in colon cancer cell lines: HCT-116 (FIG. 95), LoVo (FIG. 96), and HT-29 (FIG. 97) cells.

FIG. 98-100. Inhibitory effects of PM00104 in combination with irinotecan in colon cancer cell lines: HCT-116 (FIG. 98), LoVo (FIG. 99), and HT-29 (FIG. 100) cells.

FIG. 101. Tumor volume evaluation (mean±SEM) of NCI-H460 tumors in mice treated with control, PM00104 (0.9 mg/kg/day), bevacizumab (5 mg/kg/day) or PM00104 plus bevacizumab.

FIG. 102. Tumor volume evaluation (mean±SEM) of NCI-H460 tumors in mice treated with control, PM00104 (0.9 mg/kg/day), bevacizumab (2.5 mg/kg/day) or PM00104 plus bevacizumab.

FIG. 103. Tumor volume evaluation (mean±SEM) of NCI-H460 tumors in mice treated with control, PM00104 (0.9 mg/kg/day), temsirolimus (20 mg/kg/day) or PM00104 plus temsirolimus.

FIG. 104. Tumor volume evaluation (mean±SEM) of NCI-H460 tumors in mice treated with control, PM00104 (0.9 mg/kg/day), temsirolimus (10 mg/kg/day) or PM00104 plus temsirolimus.

FIG. 105. Tumor volume evaluation (mean±SEM) of NCI-H460 tumors in mice treated with control, PM00104 (0.9 mg/kg/day), gemcitabine (180 mg/kg/day) or PM00104 plus gemcitabine.

FIG. 106. Tumor volume evaluation (mean±SEM) of NCI-H460 tumors in mice treated with control, PM00104 (0.9 mg/kg/day), gemcitabine (90 mg/kg/day) or PM00104 plus gemcitabine.

FIG. 107. Tumor volume evaluation (mean±SEM) of CaLu-6 tumors in mice treated with control, PM00104 (0.9 mg/kg/day), gemcitabine (180 mg/kg/day) or PM00104 plus gemcitabine.

FIG. 108. Tumor volume evaluation (mean±SEM) of CaLu-6 tumors in mice treated with control, PM00104 (0.9 mg/kg/day), gemcitabine (90 mg/kg/day) or PM00104 plus gemcitabine.

FIG. 109. Tumor volume evaluation (mean±SEM) of CaLu-6 tumors in mice treated with control, PM00104 (0.9 mg/kg/day), pemetrexed (125 mg/kg/day) or PM00104 plus pemetrexed.

FIG. 110. Tumor volume evaluation (mean±SEM) of CaLu-6 tumors in mice treated with control, PM00104 (0.9 mg/kg/day), pemetrexed (100 mg/kg/day) or PM00104 plus pemetrexed.

FIG. 111. Tumor volume evaluation (mean±SEM) of NCI-H460 tumors in mice treated with control, PM00104 (0.9 mg/kg/day), pemetrexed (125 mg/kg/day) or PM00104 plus pemetrexed.

FIG. 112. Tumor volume evaluation (mean±SEM) of NCI-H460 tumors in mice treated with control, PM00104 (0.9 mg/kg/day), pemetrexed (100 mg/kg/day) or PM00104 plus pemetrexed.

FIG. 113. Tumor volume evaluation (mean±SEM) of H-Meso-1 tumors in mice treated with control, PM00104 (0.9 mg/kg/day), pemetrexed (100 mg/kg/day) or PM00104 plus pemetrexed.

FIG. 114. Tumor volume evaluation (mean±SEM) of H-Meso-1 tumors in mice treated with control, PM00104 (0.45 mg/kg/day), pemetrexed (100 mg/kg/day) or PM00104 plus pemetrexed.

### DETAILED DESCRIPTION OF THE INVENTION

We found that PM00104 greatly enhances the anticancer activity of other anticancer drugs when these anticancer drugs are combined with PM00104. Thus, the present invention is directed to provide an efficacious treatment of cancer based on the combination of PM00104, or a pharmaceutically acceptable salt thereof, with another anticancer drug.

In another aspect, the invention relates to synergistic combinations employing PM00104, or a pharmaceutically acceptable salt thereof, and another anticancer drug. Such synergistic combinations can be obtained by application of the methodology described herein, including those illustrated in Examples 1 to 24 and analyzing the results for synergistic combinations.

In the present application, by “cancer” it is meant to include tumors, neoplasias, and any other malignant disease having as cause malignant tissue or cells.

The term “treating”, as used herein, unless otherwise indicated, means reversing, alleviating, inhibiting the progress of, attenuating the symptoms or pathological basis of the disease, or preventing the disorder or condition to which such term applies, or one or more symptoms of such disorder or condition. The term “treatment”, as used herein, unless otherwise indicated, refers to the act of treating as “treating” is defined immediately above.

The term “combination” as used throughout the specification, is meant to encompass the administration to a patient suffering from cancer of the referred therapeutic agents in the same or separate pharmaceutical formulations, and at the same time or at different times. If the therapeutic agents are administered at different times they should be administered sufficiently close in time to provide for the potentiating or synergistic response to occur.

In another aspect, the invention is directed to the use of PM00104, or a pharmaceutically acceptable salt thereof, for the manufacture of a medicament for an effective treatment of cancer by combination therapy employing PM00104, or a pharmaceutically acceptable salt thereof, with another anticancer drug.

In a further aspect, the present invention is directed to a method of treating cancer comprising administering to a patient in need of such treatment a therapeutically effective amount of PM00104, or a pharmaceutically acceptable salt thereof, in combination with a therapeutically effective amount of another anticancer drug.

Depending on the type of tumor and the development stage of the disease, anticancer effects of the methods of treatment of the present invention include, but are not limited to, inhibition of tumor growth, tumor growth delay, regression of tumor, shrinkage of tumor, reduction of tumor size and/or tumor markers, increased time to regrowth of tumor on cessation of treatment, slowing of disease progression, and prevention of metastasis. It is expected that when a method of treatment of the present invention is administered to a patient, such as a human patient, in need of such treatment, said method of treatment will produce an effect, as measured by, for example, the extent of the anticancer effect, the response rate, the time to disease progression, or the survival rate. In particular, the methods of treatment of the invention are suited for human patients, especially those who are relapsing or refractory to previous chemotherapy. First line therapy is also envisaged.

As mentioned above, PM00104 is an alkaloid related to the marine compounds jorumycin and renieramycins, and also to safracin and saframycin compounds, having the following structure:

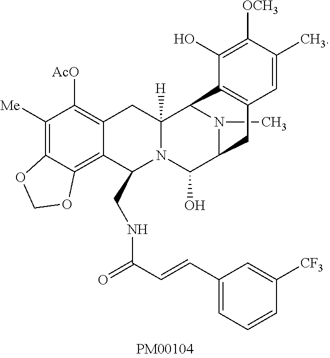

The term “PM00104” is intended here to cover any pharmaceutically acceptable salt, solvate, hydrate, prodrug, or any other compound which, upon administration to the patient is capable of providing (directly or indirectly) the compound as described herein. The preparation of salts, solvates, hydrates, and prodrugs can be carried out by methods known in the art.

Pharmaceutically acceptable salts can be synthesized from the parent compound, which contains a basic or acidic moiety, by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, trifluoroacetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium and ammonium salts, and organic alkali salts such as, for example, ethylenediamine, ethanolamine, N,N-dialkylenethanolamine, triethanolamine and basic aminoacids salts.

Any compound that is a prodrug of PM00104 is within the scope and spirit of the invention. The term “prodrug” is used in its broadest sense and encompasses those derivatives that are converted in vivo to PM00104. The prodrug can hydrolyze, oxidize, or otherwise react under biological conditions to provide PM00104. Examples of prodrugs include, but are not limited to, derivatives and metabolites of PM00104 that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well-known methods, such as those described by Burger “Medicinal Chemistry and Drug Discovery 6th ed. (Donald J. Abraham ed., 2001, Wiley) and “Design and Applications of Prodrugs” (H. Bundgaard ed., 1985, Harwood Academic Publishers).

In addition, any drug referred to herein may be in crystalline form either as free compound or as solvates (e.g. hydrates) and it is intended that both forms are within the scope of the present invention. Methods of solvation are generally known within the art.

PM00104 for use in accordance of the present invention may be prepared following the synthetic process disclosed in WO 01/87894, which is incorporated herein by reference.

Pharmaceutical compositions of PM00104 that can be used include solutions, suspensions, emulsions, lyophilised compositions, etc., with suitable excipients for intravenous administration. Preferably, PM00104 may be supplied and stored as a sterile lyophilized product, comprising PM00104 and excipients in a formulation adequate for therapeutic use. In particular a formulation comprising sucrose and a phosphate salt buffered to an adequate pH is preferred. Further guidance on PM00104 formulations is given in WO 2007/052076 which is incorporated herein by reference in its entirety.

Administration of PM00104, or pharmaceutical compositions thereof, or of pharmaceutical compositions comprising the compound is preferably by intravenous infusion. Infusion times of up to 72 hours can be used, more preferably between 1 and 24 hours, with either about 1, about 3 or about 24 hours most preferred. Short infusion times which allow treatment to be carried out without an overnight stay in hospital are especially desirable. However, infusion may be around 24 hours or even longer if required.

Preferably, the administration PM00104 is performed in cycles. In a preferred administration method an intravenous infusion of PM00104 is given to the patients typically the first day of each cycle and then the patients are allowed to recover for the remainder of the cycle. The preferred duration of each cycle is typically of 3 or 4 weeks; multiple cycles can be given as needed. Dose delays and/or dose reductions and schedule adjustments are performed as needed depending on individual patient condition and tolerance to treatments. For further guidance on PM00104 administration and dosages, see for example WO 2008/135792 which is incorporated herein by specific reference.

In the present invention, particularly preferred is the combination of PM00104, or a pharmaceutically acceptable salt thereof, with another anticancer drug in the treatment of a cancer selected from lung cancer, sarcoma, malignant melanoma, pleural mesothelioma, bladder carcinoma, prostate cancer, pancreas carcinoma, gastric carcinoma, ovarian cancer, hepatoma, breast cancer, colorectal cancer, kidney cancer, esophageal cancer, suprarenal cancer, parotid gland cancer, head & neck carcinoma, cervix cancer, mesothelioma, leukaemia, and lymphoma.

In addition, particularly preferred is the combination of PM00104, or a pharmaceutically acceptable salt thereof, with another anticancer drug selected from antitumor platinum coordination complexes, antimetabolites, mitotic inhibitors, anthracyclines, topoisomerase I and/or II inhibitors, antitumor monoclonal antibodies, mTOR inhibitors, and tyrosine kinase inhibitors in the treatment of cancer, and more particularly in the treatment of a cancer selected from lung cancer, sarcoma, malignant melanoma, pleural mesothelioma, bladder carcinoma, prostate cancer, pancreas carcinoma, gastric carcinoma, ovarian cancer, hepatoma, breast cancer, colorectal cancer, kidney cancer, esophageal cancer, suprarenal cancer, parotid gland cancer, head & neck carcinoma, cervix cancer, mesothelioma, leukaemia, and lymphoma.

In a preferred embodiment, the invention is directed to the combination of PM00104, or a pharmaceutically acceptable salt thereof, with an antitumor platinum coordination complex in the treatment of cancer, and more particularly in the treatment of a cancer selected from gastric carcinoma, bladder carcinoma, lung cancer, and colorectal cancer. This chemotherapeutic group includes, but is not limited to, cisplatin, oxaliplatin, carboplatin, BBR3464, satraplatin, tetraplatin, ormiplatin, and iproplatin. Particularly preferred is the combination of PM00104, or a pharmaceutically acceptable salt thereof, with cisplatin, oxaliplatin, carboplatin, BBR3464, satraplatin, tetraplatin, ormiplatin, and iproplatin, and even more preferred is the combination with cisplatin and oxaliplatin in the treatment of cancer, and more particularly in the treatment of a cancer selected from gastric carcinoma, bladder carcinoma, lung cancer, and colorectal cancer.

In another preferred embodiment, the invention is directed to the combination of PM00104, or a pharmaceutically acceptable salt thereof, with an antimetabolite in the treatment of cancer, and more particularly in the treatment of a cancer selected from gastric carcinoma, pancreatic carcinoma, bladder carcinoma, colorectal cancer, lung cancer, breast cancer, and mesothelioma. This chemotherapeutic group includes, but is not limited to, 5-fluorouracil, gemcitabine, cytarabine, capecitabine, decitabine, floxuridine, 6-mercaptopurine, methotrexate, fludarabine, aminopterin, pemetrexed, raltitrexed, cladribine, clofarabine, fludarabine, mercaptopurine, pentostatin, and thioguanine. Particularly preferred is the combination of PM00104, or a pharmaceutically acceptable salt thereof, with 5-fluorouracil, gemcitabine, cytarabine, capecitabine, decitabine, floxuridine, 6-mercaptopurine, methotrexate, fludarabine, aminopterin, pemetrexed, raltitrexed, cladribine, clofarabine, fludarabine, mercaptopurine, pentostatin, and thioguanine, and even more preferred is the combination with 5-fluorouracil, pemetrexed, and gemcitabine in the treatment of cancer, and more particularly in the treatment of a cancer selected from gastric carcinoma, pancreatic carcinoma, bladder carcinoma, colorectal cancer, lung cancer, breast cancer, and mesothelioma.

In another preferred embodiment, the invention is directed to the combination of PM00104, or a pharmaceutically acceptable salt thereof, with a mitotic inhibitor in the treatment of cancer, and more particularly in the treatment of a cancer selected from gastric carcinoma, bladder carcinoma, lung cancer, and breast cancer. This chemotherapeutic group includes, but is not limited to, paclitaxel, docetaxel, vinblastine, vincristine, vindesine, and vinorelbine. Particularly preferred is the combination of PM00104, or a pharmaceutically acceptable salt thereof, with paclitaxel, docetaxel, vinblastine, vincristine, vindesine, and vinorelbine, and even more preferred is the combination with paclitaxel and docetaxel in the treatment of cancer, and more particularly in the treatment of a cancer selected from gastric carcinoma, bladder carcinoma, lung cancer, and breast cancer.

In another preferred embodiment, the invention is directed to the combination of PM00104, or a pharmaceutically acceptable salt thereof, with an anthracycline in the treatment of cancer, and more particularly in the treatment of gastric carcinoma and breast cancer. This chemotherapeutic group includes, but is not limited to, daunorubicin, doxorubicin, epirubicin, idarubicin, mitoxantrone, pixantrone, and valrubicin. Particularly preferred is the combination of PM00104, or a pharmaceutically acceptable salt thereof, with aunorubicin, doxorubicin, epirubicin, idarubicin, mitoxantrone, pixantrone, and valrubicin, and even more preferred is the combination with doxorubicin in the treatment of cancer, and more particularly in the treatment of gastric carcinoma and breast cancer.

In another preferred embodiment, the invention is directed to the combination of PM00104, or a pharmaceutically acceptable salt thereof, with a topoisomerase I and/or II inhibitor in the treatment of cancer, and more particularly in the treatment of gastric carcinoma and colorectal cancer. This chemotherapeutic group includes, but is not limited to, topotecan, SN-38, irinotecan, camptothecine, rubitecan, etoposide, and teniposide. Particularly preferred is the combination of PM00104, or a pharmaceutically acceptable salt thereof, with topotecan, SN-38, irinotecan, camptothecine, rubitecan, etoposide, and teniposide, and even more preferred is the combination with SN-38 and irinotecan in the treatment of cancer, and more particularly in the treatment of gastric carcinoma and colorectal cancer.

In another preferred embodiment, the invention is directed to the combination of PM00104, or a pharmaceutically acceptable salt thereof, with antitumor monoclonal antibodies in the treatment of cancer, and more particularly in the treatment of ovarian cancer and lung cancer. This chemotherapeutic group includes, but is not limited to, bevacizumab, cetuximan, panitumumab, trastuzumab, rituximab, tositumomab, alemtuzumab, and gemtuzumab. Particularly preferred is the combination of PM00104, or a pharmaceutically acceptable salt thereof, with bevacizumab, cetuximan, panitumumab, trastuzumab, rituximab, tositumomab, alemtuzumab, and gemtuzumab, and even more preferred is the combination with bevacizumab in the treatment of cancer, and more particularly in the treatment of ovarian cancer and lung cancer.

In another preferred embodiment, the invention is directed to the combination of PM00104, or a pharmaceutically acceptable salt thereof, with a tyrosine kinase inhibitor in the treatment of cancer, and more particularly in the treatment of a cancer selected from hepatoma and pancreas carcinoma. This chemotherapeutic group includes, but is not limited to, erlotinib, sorafenib, axitinib, bosutinib, cediranib, dasatinib, gefitinib, imatinib, canertinib, lapatinib, lestaurtinib, nilotinib, semaxanib, sunitinib, and vandetanib. Particularly preferred is the combination of PM00104, or a pharmaceutically acceptable salt thereof, with erlotinib, sorafenib, axitinib, bosutinib, cediranib, dasatinib, gefitinib, imatinib, canertinib, lapatinib, lestaurtinib, nilotinib, semaxanib, sunitinib, and vandetanib, and even more preferred is the combination with erlotinib and sorafenib in the treatment of cancer, and more particularly in the treatment of a cancer selected from hepatoma and pancreas carcinoma.

In another preferred embodiment, the invention is directed to the combination of PM00104, or a pharmaceutically acceptable salt thereof, with an mTOR inhibitor in the treatment of cancer, and more particularly in the treatment of lung cancer. This chemotherapeutic group includes, but is not limited to, temsirolimus, sirolimus (rapamycin), everolimus, and deforolimus. Particularly preferred is the combination of PM00104, or a pharmaceutically acceptable salt thereof, with temsirolimus, sirolimus (rapamycin), everolimus, and deforolimus, and even more preferred is the combination with temsirolimus in the treatment of cancer, and more particularly in the treatment of lung cancer.

The invention includes any pharmaceutically acceptable salt of any drug referred to herein, which can be synthesized from the parent compound by conventional chemical methods as disclosed before.

PM00104, or a pharmaceutically acceptable salt thereof, and the other anticancer drug may be provided as separate medicaments for administration at the same time or at different times. Preferably, PM00104 and the other anticancer drug are provided as separate medicaments for administration at different times. When administered separately and at different times, either PM00104 or the other anticancer drug, may be administered first. In addition, both drugs can be administered in the same day or at different days, and they can be administered using the same schedule or at different schedules during the treatment cycle. Thus, the pharmaceutical compositions of the present invention may comprise all the components (drugs) in a single pharmaceutically acceptable formulation. Alternatively, the components may be formulated separately and administered in combination with one another. Various pharmaceutically acceptable formulations well known to those of skill in the art can be used in the present invention. Additionally, the drugs of the combination may be given using different administration routes. For instance, one of the drugs may be in a form suitable for oral administration, for example as a tablet or capsule, and the other one in a form suitable for parenteral injection (including intravenous, subcutaneous, intramuscular, intravascular or infusion), for example as a sterile solution, suspension or emulsion. Alternatively, both drugs may be given by the same administration route. Selection of an appropriate formulation for use in the present invention can be performed routinely by those skilled in the art based upon the mode of administration and the solubility characteristics of the components of the composition.

The correct dosage of the compounds of the combination will vary according to the particular formulation, the mode of application, and the particular site, patient and tumour being treated. Other factors like age, body weight, sex, diet, time of administration, rate of excretion, condition of the patient, drug combinations, reaction sensitivities and severity of the disease shall be taken into account. Administration can be carried out continuously or periodically within the maximum tolerated dose.

In another aspect, the present invention is directed to a kit for administering PM00104 in combination with another anticancer drug in the treatment of cancer, comprising a supply of PM00104, or a pharmaceutically acceptable salt thereof, in dosage units for at least one cycle, and printed instructions for the use of both drugs in combination.

In a related aspect, the present invention is directed to a kit for administering PM00104 in combination with another anticancer drug in the treatment of cancer, comprising a supply of PM00104, or a pharmaceutically acceptable salt thereof, in dosage units for at least one cycle, a supply of the other anticancer drug in dosage units for at least one cycle, and printed instructions for the use of both drugs in combination.

In another aspect, the present invention also provides a pharmaceutical composition comprising PM00104, or a pharmaceutically acceptable salt thereof, and a pharmaceutically acceptable carrier or excipient, for use in combination with another anticancer drug in the treatment of cancer.

In a further aspect, the present invention also provides a pharmaceutical composition comprising PM00104, or a pharmaceutically acceptable salt thereof, another anticancer drug, and a pharmaceutically acceptable carrier or excipient, for use in the treatment of cancer.

In another aspect, the invention further provides for the use of PM00104, or a pharmaceutically acceptable salt thereof, for the manufacture of a medicament for the treatment of cancer, in combination therapy with another anticancer.

In a related aspect, the invention further provides for the use of PM00104, or a pharmaceutically acceptable salt thereof, in combination with another anticancer drug for the manufacture of a medicament for the treatment of cancer.

In another aspect, the invention provides PM00104, or a pharmaceutically acceptable salt thereof, for the treatment of cancer comprising administering a therapeutically effective amount of PM00104, or a pharmaceutically acceptable salt thereof, in combination with a therapeutically effective amount of another anticancer drug.

In another aspect, the invention provides a method for the treatment of cancer comprising the administration of a therapeutically effective amount of PM00104, or pharmaceutically acceptable salt thereof, in combination with the administration of a therapeutically effective amount of another anticancer drug, wherein the combination may be administered together or separately. In preferred embodiments of the invention PM00104, or pharmaceutically acceptable salts thereof, and the other anticancer drugs are administered in synergistically effective amounts.

In one embodiment, cancer cells are contacted, or otherwise treated, with a combination of PM00104, or a pharmaceutically acceptable salt thereof, and another anticancer drug. The cancer cells are preferably human and include carcinoma cells, sarcoma cells, leukemia cells, and lymphoma cells. More preferably, the cancer cells are cells of lung cancer, sarcoma, malignant melanoma, pleural mesothelioma, bladder carcinoma, prostate cancer, pancreas carcinoma, gastric carcinoma, ovarian cancer, hepatoma, breast cancer, colorectal cancer, kidney cancer, esophageal cancer, suprarenal cancer, parotid gland cancer, head & neck carcinoma, cervix cancer, mesothelioma, leukaemia, and lymphoma. In particular, the cancer cells include human gastric carcinoma cells, human bladder carcinoma cells, and human pancreas carcinoma cells. In addition, the combination provides a synergistic inhibitory effect against cancer cells, particularly against human gastric carcinoma cells, human bladder carcinoma cells, human pancreas carcinoma cells, human lung cancer cells, human colorectal cancer cells, and human breast cancer cells.

For example, the combination inhibits proliferation or survival of contacted cancer cells. A lower level of proliferation or survival of the contacted cancer cells compared to the non-contacted cancer cells supports the combination of PM00104, or a pharmaceutically acceptable salt thereof, and another anticancer drug selected as being effective for treating a patient with cancer.

In another aspect, the invention provides for a method for inhibiting the growth of cancer cells comprising contacting said cancer cells with an effective amount of PM00104, or a pharmaceutically acceptable salt thereof, in combination with another anticancer drug, either together or separately.

In another aspect, the invention provides for a method for inhibiting the growth of cancer cells comprising contacting said cancer cells with a synergistic combination of PM00104, or a pharmaceutically acceptable salt thereof, and another anticancer drug, together or separately, wherein said combination provides improved inhibition against cancer cell growth as compared to (i) PM00104, or a pharmaceutically acceptable salt thereof, in the absence of another anticancer drug, or (ii) the other anticancer drug in the absence of PM00104.

In another aspect, the invention provides for a pharmaceutical composition comprising an effective amount of PM00104, or a pharmaceutically acceptable salt thereof, for use in combination with another anticancer drug for inhibiting the growth of cancer cells.

In a related aspect, the invention provides for a pharmaceutical composition comprising an effective combination of PM00104, or a pharmaceutically acceptable salt thereof, and another anticancer drug for inhibiting the growth of cancer cells.

In another aspect, the invention provides for a pharmaceutical composition comprising a synergistic combination of PM00104, or a pharmaceutically acceptable salt thereof, and another anticancer drug for inhibiting the growth of cancer cells, wherein said combination provides improved inhibition against cancer cell growth as compared to (i) PM00104, or a pharmaceutically acceptable salt thereof, in the absence of another anticancer drug, or (ii) the other anticancer drug in the absence of PM02734.

In another embodiment, the combination of PM00104, or a pharmaceutically acceptable salt thereof, and another anticancer drug inhibits tumor growth or reduce the size of a tumor in vivo. In particular, the combination inhibits in vivo growth of carcinoma cells, sarcoma cells, leukemia cells, and lymphoma cells. Preferably, the combination inhibits in vivo growth of cells of lung cancer, sarcoma, malignant melanoma, pleural mesothelioma, bladder carcinoma, prostate cancer, pancreas carcinoma, gastric carcinoma, ovarian cancer, hepatoma, breast cancer, colorectal cancer, kidney cancer, esophageal cancer, suprarenal cancer, parotid gland cancer, head & neck carcinoma, cervix cancer, mesothelioma, leukaemia, and lymphoma. In particular, the cancer cells include human gastric carcinoma cells, human bladder carcinoma cells, human pancreas carcinoma cells, human hepatoma cells, human lung cancer cells, human mesothelioma cells, and human ovary cancer cells. Similarly, the combination reduces the size of carcinoma, sarcoma, leukemia, and lymphoma tumors in vivo. Preferably, the combination reduces the size of lung cancer, sarcoma, malignant melanoma, pleural mesothelioma, bladder carcinoma, prostate cancer, pancreas carcinoma, gastric carcinoma, ovarian cancer, hepatoma, breast cancer, colorectal cancer, kidney cancer, esophageal cancer, suprarenal cancer, parotid gland cancer, head & neck carcinoma, cervix cancer, mesothelioma, leukaemia, and lymphoma. Specifically, the combination reduces the size of human hepatoma, mesothelioma, gastric, bladder, pancreas, lung, and ovary tumors in vivo.

For example, the combination inhibits tumor growth or reduces the size of human cancer xenografts, particularly human hepatoma, mesothelioma, gastric, bladder, pancreas, lung, and ovary tumors xenografts, in animal models. A reduced growth or reduced size of human cancer xenografts in animal models administered with the combination further supports the combination of PM00104, or a pharmaceutically acceptable salt thereof, and another anticancer drug as being effective for treating a patient with cancer.

According to an embodiment of the invention, tumor growth inhibition is assessed comparing the mean tumor weight of the treatment combining the two drugs (PM00104 and another drug) with those of the other drug monotherapy treatment. Additionally, the definition and criteria for the evaluation of potentiation and the degree of additivity for the combination therapy are as follows:

Potentiation can be determined when the response of the combination therapy is greater than the best response of the most active drug administered as single agent (monotherapy) on the same schedule and dose as used in the combination therapy.

Additivity is determined by comparing the % of tumor growth inhibition of the monotherapy treatments versus those of the combination treatment as follows:
1. Determination of the % of tumor growth inhibition, as 100-% T/C, for each of the drugs administered as monotherapy at the doses used in the combinations. % T/C is obtained by comparing the mean tumor weight in the treatment groups (T) to the mean tumor weight in the control group (C) (T/C×100%).
2. The two scores are added together to determine the “expected response” if each agent produced the same response as it does when administered as monotherapy.
3. This “expected response” is subtracted from the % of tumor growth inhibition determined for the combination therapy group:
   a. A negative number means that the effect of combining the two drugs is less than additive.
   b. If the resulting number is close to zero, the effect of combining the two drugs is determined as additive.
   c. A positive number means that the effect of combining the two drugs is greater than additive.

Accordingly, a greater than additive effect of the combination treatment corresponds to a synergistic effect, wherein the effect of the combination of the two drugs is therapeutically superior to that expected in view of the effect of each of the drugs when given alone.

Therefore, in another aspect, the invention provides for a method for reducing the size of a tumor, comprising administering an effective amount of PM00104, or a pharmaceutically acceptable salt thereof, in combination with another anticancer drug, either together or separately.

In another aspect, the invention provides for a method for inhibiting tumor growth, comprising administering an effective amount of PM00104, or a pharmaceutically acceptable salt thereof, in combination with another anticancer drug.

In a related aspect, the invention provides for a method for inhibiting tumor growth, comprising administering an effective combination of PM00104, or a pharmaceutically acceptable salt thereof, and an anticancer drug, either together or separately.

The following examples further illustrate the invention. The examples should not be interpreted as a limitation of the scope of the invention.

To provide a more concise description, some of the quantitative expressions given herein are not qualified with the term “about”. It is understood that, whether the term “about” is used explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value.

### EXAMPLES

### Example 1

In vitro studies to determine the effect of PM00104 in combination with chemotherapeutic agents on human gastric carcinoma cell lines.

The objective of this study was to determine the ability of PM00104 to potentiate the antitumor activity of chemotherapeutic agents used in the treatment of gastric carcinoma.

The following agents were evaluated in combination with PM00104: cisplatin, 7-ethyl-10-hydroxycamptothecin (SN38), 5-fluorouracil (5-FU), doxorubicin, docetaxel, and oxaliplatin. The human gastric carcinoma cell lines selected for this assay were the following: Hs746T, HGC-27, and AGS cell lines. Hs746T and AGS cell lines were grown in Dulbecco's modified Eagle's medium (DMEM) supplemented with 10% Fetal Bovine Serum (FBS), 1.5 g/L sodium bicarbonate, 4.5 g/L glucose and 4 mM L-glutamine. HGC-27 cell line was grown in Iscove's modified Dulbecco's medium (IMDM) supplemented with 20% FBS and 2 mM L-glutamine.

The screening was performed in two parts:

a. In the first set of assays, IC₅₀ values were determined for each drug after 72 hours of drug exposure in each of the tumor cell lines.

All cell lines were maintained in their respective growth medium at 37° C., 5% CO₂ and 98% humidity. The growth medium formulations did not contain antibiotic. The day before plating, the cells were fed with fresh, complete growth media. On the harvest (plating) day, cells were counted by Trypan Blue exclusion staining method.

Cells were harvested and seeded in 96 well microtiter plates at 10,000 cells density in 150 μL of media and incubated for 24 hours to allow the cells to attach before drug addition. To collect reference data, the MTS assay was done on untreated cells at time 0 (after incubation of cells for 24 hours).

Stock solutions of PM00104, cisplatin, SN38, and 5-FU were prepared just prior to addition to plates in 100% DMSO at 2.0 mg/mL. Stock solutions of doxorubicin and oxaliplatin were prepared in sterile water for tissue culture at 2.0 mg/mL for both drugs. Stock solution of docetaxel was prepared in ethanol at 2.0 mg/mL. Additional serial dilutions were prepared in serum-free RPMI 1640 medium to achieve a final 4× treatment concentration. 50 μL of each diluted test articles was added per well.

The cytotoxic effect was measured by the MTS Assay (Tetrazolium), which is a colorimetric method for determining the number of viable cells. After the incubation period (24 hours for the Day 0 plate and 72 hours for the Test and Control plates), 25 μL of MTS+PMS solution was added to each microtiter well and incubated for 4 hours at 37° C. Plates were then removed from incubator and placed on plate shaker for 5 minutes (covered with aluminum foil for protection from light). Optical densities were read at 490 nm on spectrophotometer plate reader.

IC₅₀ values were calculated from an average of two to four assays for each of the test agents. A regression curve using SoftMax program was generated, and then 50% inhibition concentration (mg/mL) was manually interpolated.

The individual IC₅₀ values of each agent for each cell line are shown in table 1.

TABLE 1
IC₅₀ values in mg/mL for each of the agent

| Cell line | PM00104 | Cisplatin | SN38 | 5-FU |
|---|---|---|---|---|
| Hs746T | 5.34E−06 | 3.92E−02 | 5.82E−05 | 5.46E−03 |
| AGS | 5.21E−06 | 2.55E−02 | 5.49E−06 | 5.32E−04 |
| HGC-27 | 3.71E−06 | 2.56E−02 | 2.46E−04 | 4.34E−04 |

| Cell line | Doxorubicin | Docetaxel | Oxaliplatin |
|---|---|---|---|
| Hs746T | 8.97E−03 | 4.42E−02 | 8.14E−03 |
| AGS | 1.56E−04 | 1.99E−03 | 2.89E−04 |
| HGC-27 | 1.77E−04 | 6.54E−03 | 1.92E−04 |

b. In a second set of assays, each cell line was incubated with PM00104 in combination with each of the agents mentioned above in the following combination of unique IC₅₀ concentrations:

| IC₅₀ of PM00104 | IC₅₀ of Agent |
|---|---|
| 100% | 0% |
| 75% | 25% |
| 70% | 30% |
| 60% | 40% |
| 50% | 50% |
| 40% | 60% |
| 30% | 70% |
| 25% | 75% |
| 0% | 100% |
| 0% | 0% |

Cell culture and cell plating were performed as described before. Stock solutions of each drug were also prepared as described before at a drug concentration of 1.0 mg/mL. These stock solutions were serially diluted further as needed to reach the starting concentration. Additional serial dilutions were prepared in serum-free RPMI 1640 medium to achieve a final 8× treatment concentration. 25 μL of each diluted test articles was added per well.

The cytotoxic effect was measured by the MTS Assay as described above. Data was analyzed as follows:

1. Prism (Graphpad) software program was used to normalize the data to control values (100%=cell growth in the absence of agent (drug); 0%=blank control).

2. Normalized data were plotted as x/y graphs. A line was drawn connecting the values of 100% IC₅₀ for each agent (drug). Values significantly above the line indicated antagonism, below indicated synergy, and on the line indicated additivity.

Synergistic cytotoxicity to tumor cells is an optimal effect and implies that the combination of PM00104 with another drug is more effective than either drug alone. A statistically significant observation requires that a difference exists between the combination (PM00104+another drug) absorbance value and both endpoint values (PM00104 and the other drug alone). If the majority of the values are statistically above or below the line (endpoints) then antagonism or synergy is described, respectively, otherwise the pattern is more consistent with an additive interaction.

According to this assay it was found that:

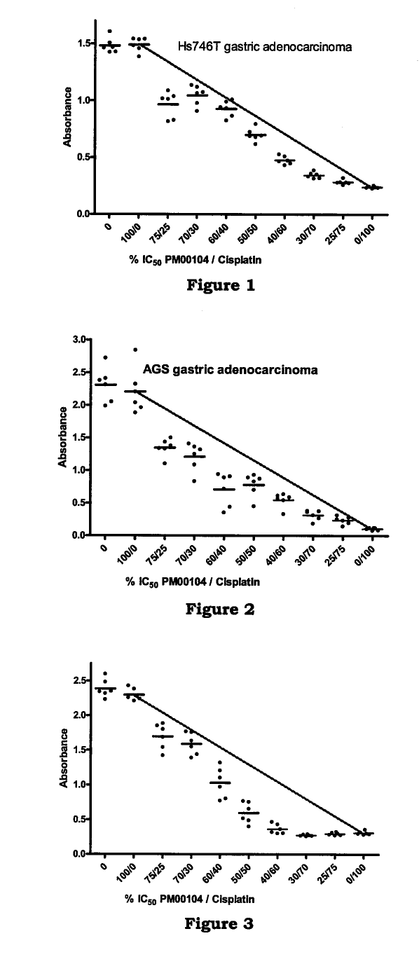

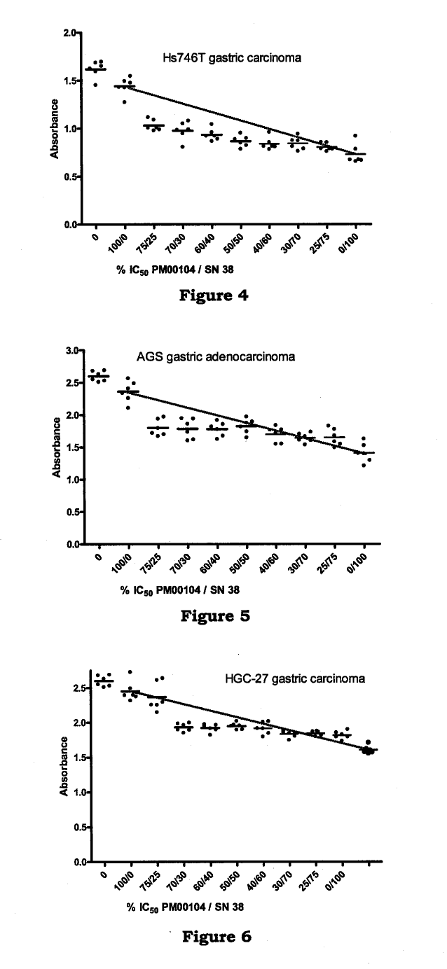

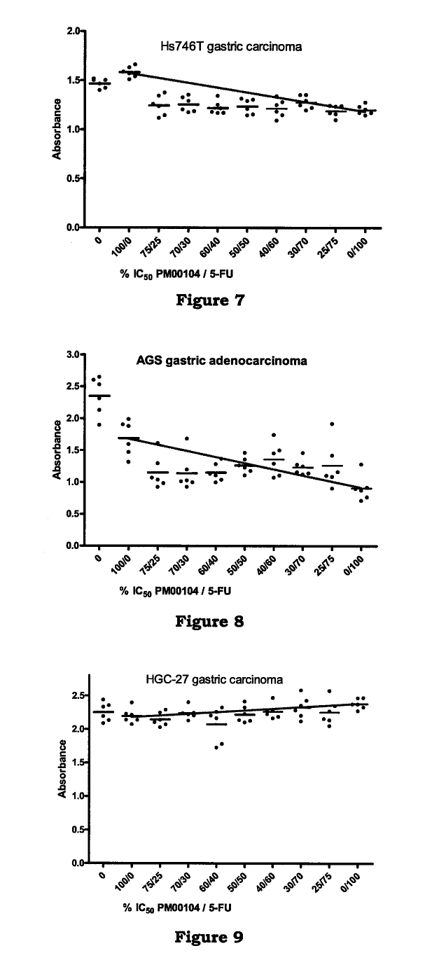

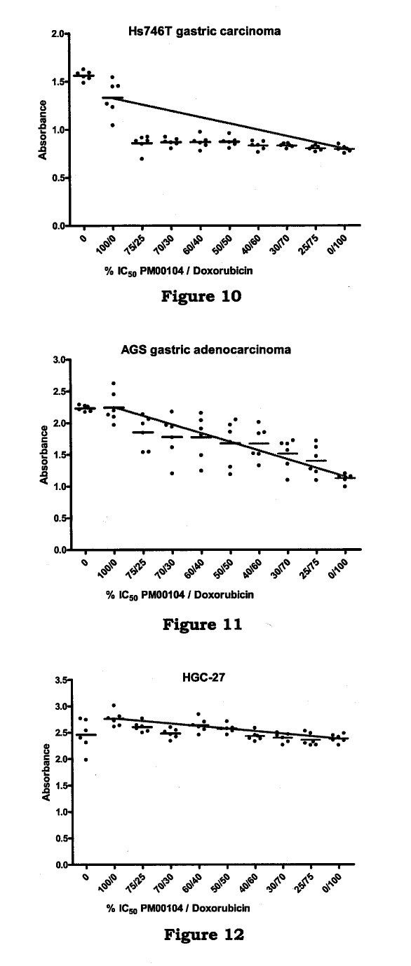

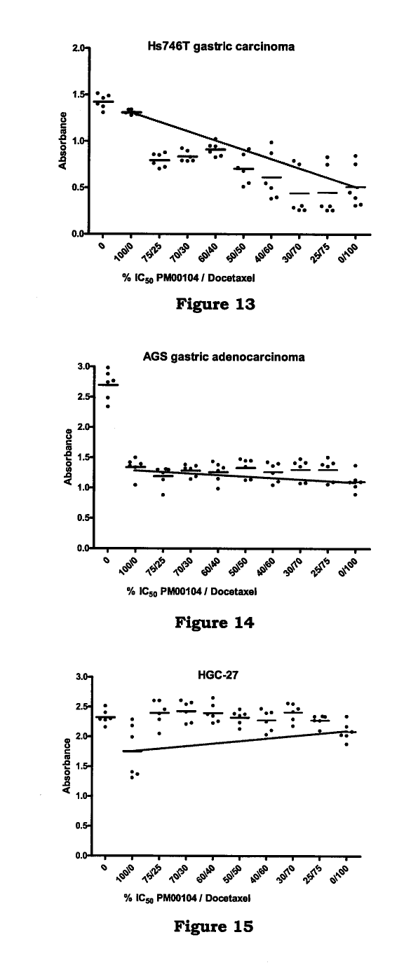

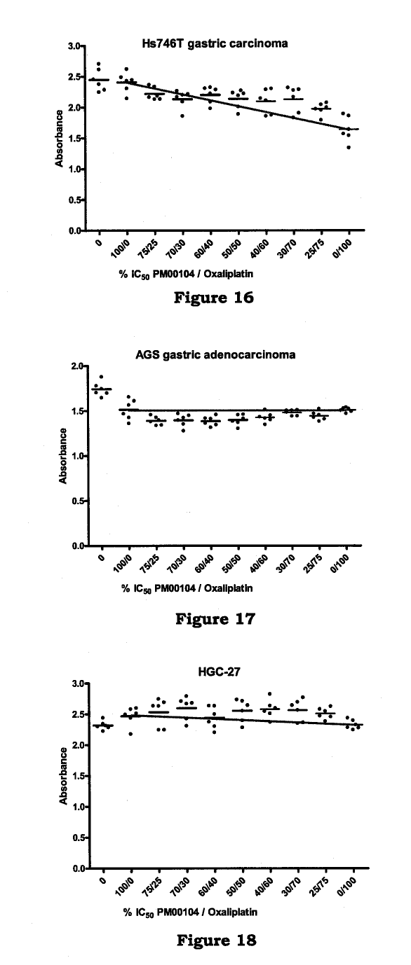

a. The combination of PM00104 with cisplatin in human gastric carcinoma cells was synergistic in Hs746T (FIG. 1), AGS (FIG. 2) and HGC-27 (FIG. 3) cell lines at almost all dose ratios.
b. The combination of PM00104 with SN38 in Hs746T cell line (FIG. 4) was synergistic at most of dose ratios, and it showed a synergistic trend in AGS cell line (FIG. 5) at the 75/25-60/40 dose ratios, and in HGC-27 cell line (FIG. 6) at the 70/30 and 60/40 dose ratios.
c. The combination of PM00104 with 5-FU showed a synergistic trend in Hs746T cell line (FIG. 7) at the 75/25-40/60 dose ratios, and in AGS cell line (FIG. 8) at the 75/25-60/40 dose ratios. In HGC-27 cell line (FIG. 9), the combination showed an additive trend.
d. The combination of PM00104 with doxorubicin was synergistic in Hs746T cell line (FIG. 10) at almost all dose ratios, and it showed an additive trend in AGS (FIG. 11) and HGC-27 (FIG. 12) cell lines.
e. The combination of PM00104 with docetaxel was synergistic in Hs746T cell line (FIG. 13), and it showed an additive trend in AGS cell line (FIG. 14) and an antagonistic trend in HGC-27 cell line (FIG. 15).
f. The combination of PM00104 with oxaliplatin showed an additive trend in Hs746T (FIG. 16), AGS (FIG. 17) and HGC-27 (FIG. 18) cell lines.

### Example 2

In vitro studies to determine the effect of PM00104 in combination with chemotherapeutic agents on human bladder carcinoma cell lines.

The objective of this study was to determine the ability of PM00104 to potentiate the antitumor activity of chemotherapeutic agents used in the treatment of bladder carcinoma.

The following agents were evaluated in combination with PM00104: gemcitabine (Gemzar®) and cisplatin. The human bladder carcinoma cell lines selected for this assay were the following: 5637 and UM-UC-3 cell lines. 5637 cell line was grown in RPMI 1640 medium supplemented with 10% FBS, 1.5 g/L sodium bicarbonate, 4.5 g/L glucose, 10 mM HEPES, 1 mM sodium pyruvate, and 2 mM L-glutamine. UM-UC-3 cell line was grown in MEM Eagle's medium supplemented with 10% FBS and 2 mM L-glutamine.

The screening was performed in two parts as disclosed in Example 1:

a. In the first set of assays, IC₅₀ values were determined for each drug after 72 hours of drug exposure in each of the tumor cell lines. It was used the same methodology as those disclosed in Example 1.

Stock solutions of PM00104 and cisplatin were prepared in 100% DMSO at 2.0 mg/mL. Stock solution of gemcitabine was prepared in sterile water for tissue culture at 2.0 mg/mL. Additional serial dilutions were prepared in serum-free RPMI 1640 medium to achieve a final 4× treatment concentration. 50 μL of each diluted test articles was added per well.

IC₅₀ values were calculated from an average of three to four assays for each of the test agents. The individual IC₅₀ values of each agent for each cell line are shown in table 2.

TABLE 2
IC₅₀ values in mg/mL for each of the agent

| Cell line | PM00104 | Gemcitabine | Cisplatin |
|---|---|---|---|
| 5637 | 5.6E−06 | 1.9E−05 | 2.4E−03 |
| UM-UC-3 | 6.9E−06 | 2.2E−05 | 5.3E−04 |

b. In a second set of assays, each cell line was incubated with PM00104 in combination with each of the agents mentioned above in the same dose ratios as those disclosed in Example 1.

The methodology used in this second part of the screening was the same as those disclosed in Example 1.

Stock solutions of each drug were also prepared as mentioned before at a drug concentration of 2.0 mg/mL. These stock solutions were serially diluted further as needed to reach the starting concentration. Additional serial dilutions were prepared in serum-free RPMI 1640 medium to achieve a final 8× treatment concentration. 25 μL of each diluted test articles was added per well.

According to this assay it was found that:

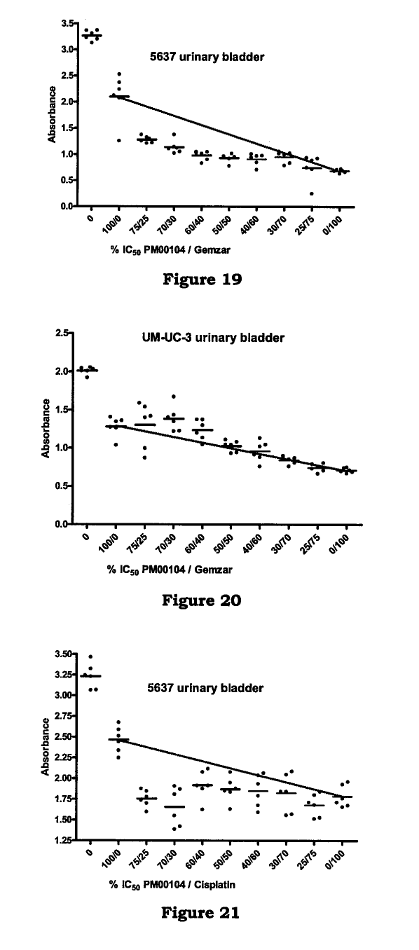

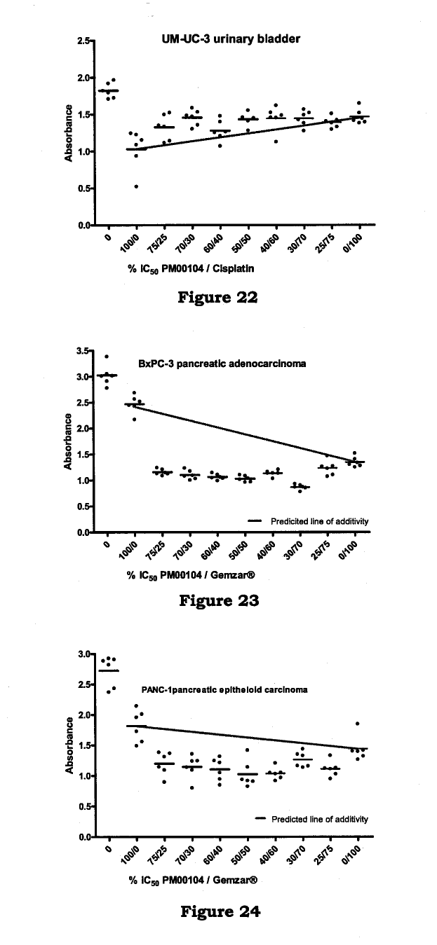

a. The combination of PM00104 with gemcitabine in human bladder carcinoma cells was synergistic in 5637 cell line (FIG. 19), and showed an additive trend in UM-UC-3 cell line (FIG. 20).
b. The combination of PM00104 with cisplatin was synergistic in 5637 cell line (FIG. 21), and showed an additive trend in UM-UC-3 cell line (FIG. 22) at the 60/40, 30/70, and 25/75 dose ratios.

### Example 3

In vitro studies to determine the effect of PM00104 in combination with chemotherapeutic agents on human pancreatic carcinoma cell lines.

The objective of this study was to determine the ability of PM00104 to potentiate the antitumor activity of chemotherapeutic agents used in the treatment of pancreatic carcinoma.

Gemcitabine (Gemzar®) was the agent evaluated in combination with PM00104. The human pancreatic carcinoma cell lines selected for this assay were the following: BxPC-3, PANC-1, MIA PaCA-2, and SW1990 cell lines. BxPC-3 cell line was grown in RPMI 1640 medium supplemented with 10% FBS, 1.5 g/L sodium bicarbonate, 4.5 g/L glucose, 10 mM HEPES, 1 mM sodium pyruvate, and 2 mM L-glutamine. PANC-1 cell line was grown in DMEM supplemented with 10% FBS, 1.5 g/L sodium bicarbonate, 4.5 g/L glucose, and 4 mM L-glutamine. MIA PaCA-2 cell line was grown in DMEM supplemented with 10% FBS, 1.5 g/L sodium bicarbonate, 4.5 g/L glucose, 2.5% Horse Serum, and 2 mM L-glutamine. SW1990 cell line was grown in RPMI 1640 medium supplemented with 10% FBS and 2 mM L-glutamine.

The screening was performed in two parts as disclosed in Example 1:

a. In the first set of assays, IC₅₀ values were determined for each drug after 72 hours of drug exposure in each of the tumor cell lines. It was used the same methodology as those disclosed in Example 1.

Stock solution of PM00104 was prepared in 100% DMSO at 2.0 mg/mL. Stock solution of gemcitabine was prepared in sterile water for tissue culture at 2.0 mg/mL. Additional serial dilutions were prepared in serum-free RPMI 1640 medium to achieve a final 4× treatment concentration. 50 μL of each diluted test articles was added per well.

IC₅₀ values were calculated from an average of three assays for each of the test agents. The individual IC₅₀ values of each agent for each cell line are shown in table 3.

TABLE 3
IC₅₀ values in mg/mL for each of the agent

| Cell line | PM00104 | Gemcitabine |
|---|---|---|
| BxPC-3 | 3.6E−05 | 8.4E−04 |
| PANC-1 | 1.5E−05 | 1.1E−01 |
| MIA PaCA-2 | 9.3E−05 | 7.9E−05 |
| SW1990 | 5.1E−06 | 1.2E−05 |

b. In a second set of assays, each cell line was incubated with PM00104 in combination with gemcitabine in the same dose ratios as those disclosed in Example 1.

The methodology used in this second part of the screening was the same as those disclosed in Example 1

Stock solutions of each drug were also prepared as mentioned before at a drug concentration of 2.0 mg/mL. These stock solutions were serially diluted further as needed to reach the starting concentration. Additional serial dilutions were prepared in serum-free RPMI 1640 medium to achieve a final 8× treatment concentration. 25 μL of each diluted test articles was added per well.

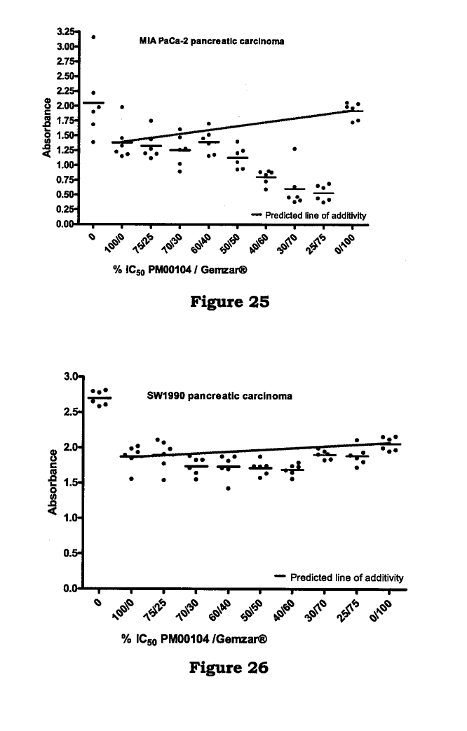

According to this assay it was found that the combination of PM00104 with gemcitabine in human pancreatic carcinoma cells was synergistic in all cell lines (BxPC-3 (FIG. 23), PANC-1 (FIG. 24), MIA PaCA-2 (FIG. 25), and SW1990 (FIG. 26) cell lines.

### Example 4

In vivo studies to determine the effect of PM00104 in combination with erlotinib and gemcitabine in human pancreas tumor xenografts.

The aim of these studies was to evaluate the ability of PM00104 to potentiate the antitumor activity of erlotinib and gemcitabine by using a xenograft model of human pancreatic carcinoma.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation with a tumor cell suspension. The Vehicle Control group contained 15 mice and the treated groups had each 10 mice/group.

The tumor model used in these studies was MIAPaCA-2 cell line, which was obtained from the ATCC (Manassas, Va.).

MIA PaCA-2 cells were grown in DMEM supplemented with 10% FBS, 1.5 g/L sodium bicarbonate, 4.5 g/L glucose, 2.5% Horse Serum, and 4 mM L-glutamine. Each animal was implanted subcutaneously (SC) on the right flank, using a trochar, with 1×10⁷ MIAPaCA-2 cells, from in vitro passage 18, in a 0.2 mL suspension of 50% Matrigel/50% serum free medium, without antibiotics. Matrigel is a biological extracellular matrix that is liquid at 4° C. and solid at 37° C., and it promotes tumor growth by maintaining the cells in close association in a localized area. Bacterial cultures were performed on aliquots of the cells prepared for implantation. All cultures were negative for bacterial contamination at both 24 and 48 hours post-implant.

Tumor measurements were determined by using Vernier calipers. The formula to calculate volume for a prolate ellipsoid was used to estimate tumor volume (mm³) from 2-dimensional tumor measurements: Tumor volume (mm³)=[L×W²]÷2, where L is the length and it is the longest diameter in mm, and W is the width and it is the shortest diameter in mm of a tumor. Assuming unit density, volume was converted to weight (i.e., 1 mm³=1 mg). When tumors reached an appropriated volume, within the size range of 175±100 mm³ (mean±SD), mice were randomized into the treatment and control groups based on tumor weight by using LabCat® In Life module V 8.0 SP1 tumor tracking and measurement software.

Treatments were initiated on DPI (Day Post Implantation) 13. In these experiments, the combination therapy groups were treated by co-administering the two drugs at the same time, with no attempt to sequence the treatments.

PM00104 was provided in the form of vials of lyophilized PM00104 powder which was reconstituted with water for injection. Erlotinib was provided in the form of a tablet and was dissolved in 0.5% carboximethylcellulose/0.4% Tween-80/Saline. Gemcitabine was provided in the form of a solid white powder containing Gemcitabine HCl, which was reconstituted in 0.9% saline.

Study groups and treatment regimens are listed in table 4.

TABLE 4

| Group | Dose | Route | Schedule | Test material |
|---|---|---|---|---|
| G1 | 10 ml/kg/day | IV | A | 0.18% Placebo in Saline |
| (Control | | | | |
| group) | | | | |
| G2 | 0.90 mg/kg/day | IV | A | PM00104 |
| G3 | 100 mg/kg/day | PO | B | erlotinib |
| G4 | 50 mg/kg/day | PO | B | erlotinib |
| G5 | 140 mg/kg/day | IP | A | gemcitabine |
| G6 | 0.90 mg/kg/day | IV | A | PM00104 |
| | 140 mg/kg/day | IP | A | gemcitabine |
| G7 | 0.90 mg/kg/day | IV | A | PM00104 |
| | 100 mg/kg/day | PO | B | erlotinib |
| G8 | 0.90 mg/kg/day | IV | A | PM00104 |
| | 50 mg/kg/day | PO | B | erlotinib |

IP: Intraperitoneal administration;
PO: Oral administration;
IV: Intravenous administration
A: DPI 13, 20, and 27;
B: DPI 13-16, 19-23, 26-30, 33-36
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and erlotinib or PM00104 and gemcitabine) against erlotinib or gemcitabine mean tumor weight, respectively, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were as follows:

Potentiation was determined when the response of the combination group was greater than the best response of the most active agent administered as single agent (monotherapy) on the same schedule and dose as used in the combination therapy.

Additivity was determined as discussed above by comparing the % of tumor growth inhibition of the monotherapy groups versus those of the combination group as follows:
1. Determine the % of tumor growth inhibition, as 100-% T/C, for each of the drugs administered as monotherapy at the doses used in the combinations. % T/C was obtained by comparing the mean tumor weight in the treatment groups (T) to the mean tumor weight in the control group (C) (T/C×100%).
2. The two scores were added together to determine the “expected response” if each agent produced the same response as it did when administered as monotherapy.
3. This “expected response” was subtracted from the % of tumor growth inhibition determined for the combination therapy group:
   a. A negative number meant that the effect of combining the two drugs was less than additive.
   b. If the resulting number was close to zero, the effect of combining the two drugs was determined as additive.
   c. A positive number meant that the effect of combining the two drugs was greater than additive.

Accordingly, a greater than additive effect of the combination treatment corresponds to a synergistic effect, wherein the effect of the combination of the two drugs is therapeutically superior to that expected in view of the effect of each of the drugs when given alone.

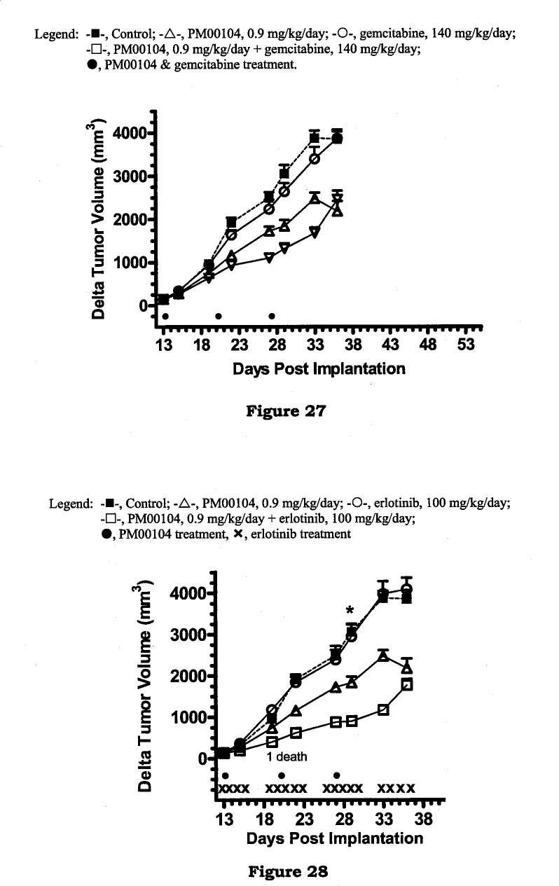

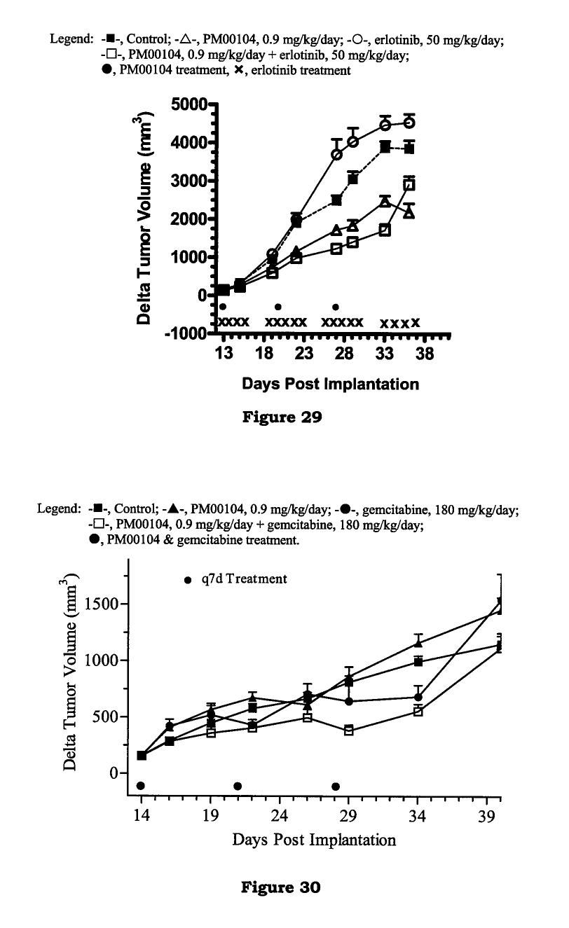

Table 5 reports the % T/C values obtained with each of the treatments and FIGS. 27-29 show the tumor volume evaluation (mean±SEM) of MIAPaCA-2 tumors in mice treated with control (vehicle), PM00104, gemcitabine, PM00104 plus gemcitabine, or PM00104 plus erlotinib at different doses.

TABLE 5

| | % T/C on day | | | | | | | |
|---|---|---|---|---|---|---|---|---|
| Group | 13 | 15 | 19 | 22 | 27 | 29 | 33 | 36 |
| G1 | — | — | — | — | — | — | — | — |
| (Control group) | | | | | | | | |
| G2 | 99.1 | 87.1 | 77.2 | 60.8 | 69.2 | 60.0 | 63.9 | 56.6 |
| G3 | 103.3 | 115.4 | 122.5 | 96.4 | 96.0 | 96.4 | 102.7 | 106.0 |
| G4 | 101.5 | 96.7 | 113.2 | 104.4 | 148.6 | 132.0 | 115.3 | 117.7 |
| G5 | 100.0 | 105.7 | 97.0 | 85.5 | 89.5 | 86.3 | 87.7 | 100.3 |
| G6 | 101.0 | 82.7 | 66.0 | 48.8 | 44.2 | 43.2 | 43.3 | 63.7 |
| G7 | 96.9 | 61.7 | 42.1 | 32.7 | 35.3 | 29.5 | 30.3 | 46.0 |
| G8 | 100.2 | 72.6 | 62.3 | 51.6 | 49.7 | 46.1 | 44.3 | 75.7 |

Table 6 shows the % of tumor growth inhibition of PM00104 and gemcitabine administered as single agents and in combination at a dose of 0.9 mg/kg/day of PM00104 and 140 mg/kg/day of gemcitabine. Additionally, the potentiation and the degree of additivity of the combination of PM00104 with gemcitabine at said doses are provided.

TABLE 6

| | % Inhibition | | | Expected | Actual | | Degree of |
|---|---|---|---|---|---|---|---|
| Day | G2 | G5 | G6 | Response | Response | Potentiation | Response |
| 13 | 0.9 | 0.0 | −1.0 | 0.94 | −1.92 | no | — |
| 15 | 12.9 | −5.7 | 17.3 | 7.13 | 10.2 | yes | Greater than additive |
| 19 | 22.8 | 3.0 | 34.0 | 25.85 | 8.18 | yes | Additive |
| 22 | 39.2 | 14.5 | 51.2 | 53.75 | −2.51 | yes | Additive |
| 27 | 30.8 | 10.5 | 55.8 | 41.27 | 14.5 | yes | Greater than additive |
| 29 | 40.0 | 13.7 | 56.8 | 53.69 | 3.15 | yes | Additive |
| 33 | 36.1 | 12.3 | 56.7 | 48.45 | 8.29 | yes | Greater than additive |
| 36 | 43.4 | −0.3 | 36.3 | 43.07 | −6.77 | no | — |

Table 7 shows the % of tumor growth inhibition of PM00104 and erlotinib administered as single agents and in combination at a dose of 0.9 mg/kg/day of PM00104 and 100 mg/kg/day of erlotinib. Additionally, the potentiation and the degree of additivity of the combination of PM00104 with erlotinib at said doses are provided.

TABLE 7

| | % Inhibition | | | Expected | Actual | | Degree of |
|---|---|---|---|---|---|---|---|
| Day | G2 | G3 | G7 | Response | Response | Potentiation | Response |
| 13 | 0.9 | −3.3 | 3.1 | −2.37 | 5.47 | yes | Additive |
| 15 | 12.9 | −15.4 | 38.3 | −2.54 | 40.84 | yes | Greater than additive |
| 19 | 22.8 | −22.5 | 57.9 | 0.37 | 57.53 | yes | Greater than additive |
| 22 | 39.2 | 3.6 | 67.3 | 42.85 | 24.45 | yes | Greater than additive |
| 27 | 30.8 | 4.0 | 64.7 | 34.76 | 29.94 | yes | Greater than additive |
| 29 | 40.0 | 3.7 | 70.5 | 43.61 | 26.89 | yes | Greater than additive |
| 33 | 36.1 | −2.7 | 69.7 | 33.44 | 36.26 | yes | Greater than additive |
| 36 | 43.4 | −6.0 | 54.0 | 37.37 | 16.63 | yes | Greater than additive |

Table 8 shows the % of tumor growth inhibition of PM00104 and erlotinib administered as single agents and in combination at a dose of 0.9 mg/kg/day of PM00104 and 50 mg/kg/day of erlotinib. Additionally, the potentiation and the degree of additivity of the combination of PM00104 with erlotinib at said doses are provided.

TABLE 8

| | % Inhibition | | | Expected | Actual | | Degree of |
|---|---|---|---|---|---|---|---|
| Day | G2 | G4 | G8 | Response | Response | Potentiation | Response |
| 13 | 0.9 | −1.5 | −0.2 | −0.56 | 0.36 | no | — |
| 15 | 12.9 | 3.3 | 27.4 | 16.2 | 11.2 | yes | Greater than additive |
| 19 | 22.8 | −13.2 | 37.7 | 9.62 | 28.08 | yes | Greater than additive |
| 22 | 39.2 | −4.4 | 48.4 | 34.78 | 13.62 | yes | Greater than additive |
| 27 | 30.8 | −48.6 | 50.3 | −17.82 | 68.12 | yes | Greater than additive |
| 29 | 40.0 | −32.0 | 53.9 | 7.94 | 45.96 | yes | Greater than additive |
| 33 | 36.1 | −15.4 | 55.7 | 20.76 | 34.94 | yes | Greater than additive |
| 36 | 43.4 | −17.7 | 24.3 | 25.69 | −1.39 | no | — |

According to this assay it was found that:

a. The combination of PM00104 and gemcitabine resulted in a statistically significant (p≦0.001) antitumor activity compared to the control group. This combination therapy produced a statistically significant (p<0.001) potentiation of activity over results obtained with the single agent groups. At the end of the treatment this potentiation was determined to be greater than additive.
b. The combination of PM00104 and erlotinib (at both doses of erlotinib) resulted in a statistically significant (p≦0.001) antitumor activity compared to the control group as well as a statistically significant (p<0.001) potentiation of activity over results obtained. This potentiation was determined to be greater than additive.

### Example 5

In vivo studies to determine the effect of PM00104 in combination with gemcitabine in human pancreas tumor xenografts.

The aim of these studies was to evaluate the ability of PM00104 to potentiate the antitumor activity of gemcitabine by using a xenograft model of human pancreatic carcinoma.

Female CB17.SCID mice (Charles River Lab.) were utilized for all experiments. Animals were housed in ventilated rack caging with food and water ad libitum. The mice were acclimated for at least 5 days prior to tumor implantation with a tumor cell suspension. The Vehicle Control group contained 15 mice and the treated groups had each 10 mice/group.

The tumor model used in these studies was BxPC-3 cell line, which was obtained from the ATCC (Manassas, Va.).

BxPC-3 cells were grown in complete RPMI 1640 supplemented with 10% FBS and L-glutamine, without antibiotic. Each animal was implanted SC on the right flank, using a 13G trochar and 1 cc syringe, with 1×10⁷ BxPC-3 cells, from in vitro passage 12, in a 0.2 mL suspension of Matrigel and RPMI 1640 serum free medium, without antibiotics. Bacterial cultures were performed on aliquots of the cells prepared for implantation. All cultures were negative for bacterial contamination at both 24 and 48 hours post-implant.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 14.

PM00104 was provided in the form of vials of lyophilized PM00104 powder which was reconstituted with water for injection. Gemcitabine was provided in the form of a solid white powder containing Gemcitabine HCl, which was reconstituted in 0.9% saline.

Study groups and treatment regimens are listed in table 9.

TABLE 9

| Group | Dose | Route | Schedule | Test material |
|---|---|---|---|---|
| G1 | 10 ml/kg/day | IV | A | 0.18% Placebo |
| (Control group) | | | | in Saline |
| G2 | 0.90 mg/kg/day | IV | A | PM00104 |
| G3 | 180 mg/kg/day | IP | A | gemcitabine |
| G4 | 0.90 mg/kg/day | IV | A | PM00104 |
| | 180 mg/kg/day | IP | A | gemcitabine |

A: DPI 14, 21, and 28;
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and gemcitabine) against gemcitabine mean tumor weight.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups. The definition and criteria for the evaluation of potentiation and the degree of additivity for the combination therapy were the same as those disclose in Example 4.

Table 10 reports the % T/C values obtained with each of the treatments and FIG. 30 shows the tumor volume evaluation (mean±SEM) of BxPC-3 tumors in mice treated with control (vehicle), PM00104, gemcitabine, and PM00104 plus gemcitabine.

TABLE 10

| | % T/C on day | | | | | | | |
|---|---|---|---|---|---|---|---|---|
| Group | 14 | 16 | 19 | 22 | 26 | 29 | 34 | 40 |
| G1 | — | — | — | — | — | — | — | — |
| (Control group) | | | | | | | | |
| G2 | 100.9 | 140.3 | 126.1 | 116.5 | 92.2 | 106.3 | 116.8 | 126.3 |
| G3 | 96.9 | 144.6 | 115.6 | 74.5 | 106.1 | 79.3 | 68.7 | 133.9 |
| G4 | 98.3 | 98.1 | 79.7 | 70.1 | 74.7 | 46.9 | 55.8 | 96.9 |

Table 11 shows the % of tumor growth inhibition of PM00104 and gemcitabine administered as single agents and in combination at a dose of 0.9 mg/kg/day of PM00104 and 180 mg/kg/day of gemcitabine. Additionally, the potentiation and the degree of additivity of the combination of PM00104 with gemcitabine at said doses are provided.

TABLE 11

| | | | | | | Po- | |
|---|---|---|---|---|---|---|---|
| | % Inhibition | | | Expected | Actual | tenti- | Degree of |
| Day | G2 | G3 | G4 | Response | Response | ation | Response |
| 14 | −0.9 | 3.1 | 1.7 | 2.2 | −0.5 | no | — |
| 16 | −40.3 | −44.6 | 1.9 | −84.9 | 86.8 | yes | Greater than |
| | | | | | | | additive |
| 19 | −26.1 | −15.6 | 20.3 | −41.7 | 62 | yes | Greater than |
| | | | | | | | additive |
| 22 | −16.5 | 25.5 | 29.9 | 9 | 20.9 | yes | Greater than |
| | | | | | | | additive |
| 26 | 7.8 | −6.1 | 25.3 | 1.7 | 23.6 | yes | Greater than |
| | | | | | | | additive |
| 29 | −6.3 | 20.7 | 53.1 | 14.4 | 38.7 | yes | Greater than |
| | | | | | | | additive |
| 34 | −16.8 | 31.3 | 44.2 | 14.5 | 29.7 | yes | Greater than |
| | | | | | | | additive |
| 40 | −26.3 | −33.9 | 3.1 | −60.2 | 63.3 | yes | Greater than |
| | | | | | | | additive |

According to this assay it was found that the combination of PM00104 and gemcitabine resulted in a statistically significant (p≦0.05) antitumor activity compared to the control group. In addition, the combination therapy produced a potentiation of activity greater than additive.

### Example 6

In vivo studies to determine the effect of PM00104 in combination with erlotinib in human pancreas tumor xenografts.

The aim of these studies was to evaluate the ability of PM00104 to potentiate the antitumor activity of erlotinib by using a xenograft model of human pancreatic carcinoma.

Female CB17.SCID mice (Charles River Lab.) were utilized for all experiments. Animals were housed in ventilated rack caging with food and water ad libitum. The mice were acclimated for at least 5 days prior to tumor implantation with a tumor cell suspension. The Vehicle Control group contained 15 mice and the treated groups had each 10 mice/group.

The tumor model used in these studies was BxPC-3 cell line, which was obtained from the ATCC (Manassas, Va.). This cell line was grown and implanted to the animals as described in Example 5.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 9.

PM00104 was provided in the form of vials of lyophilized PM00104 powder which was reconstituted with water for injection. Erlotinib was provided in the form of a tablet and was dissolved in 0.5% Carboximethylcellulose/0.4% Tween-80/Saline (CTS).

Study groups and treatment regimens are listed in table 12.

TABLE 12

| Group | Dose | Route | Schedule | Test material |
|---|---|---|---|---|
| G1 | 10 ml/kg/day | PO | A | CTS |
| (Control group) | 10 ml/kg/day | IV | B | 0.18% Placebo |
| | | | | in Saline |
| G2 | 0.90 mg/kg/day | IV | B | PM00104 |
| G3 | 50 mg/kg/day | PO | A | erlotinib |
| G4 | 30 mg/kg/day | PO | A | erlotinib |
| G5 | 15 mg/kg/day | PO | A | erlotinib |
| G6 | 0.90 mg/kg/day | IV | B | PM00104 |
| | 50 mg/kg/day | PO | A | erlotinib |
| G7 | 0.90 mg/kg/day | IV | B | PM00104 |
| | 30 mg/kg/day | PO | A | erlotinib |
| G8 | 0.90 mg/kg/day | IV | B | PM00104 |
| | 15 mg/kg/day | PO | A | erlotinib |

A: Cycle 1 = DPI 9-13; Cycle 2 = DPI 16-20; Cycle 3 = DPI 23-27
B: DPI 9, 16, and 23
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and erlotinib) against erlotinib mean tumor weight, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups. The definition and criteria for the evaluation of potentiation and the degree of additivity for the combination therapy were the same as those disclose in Example 4.

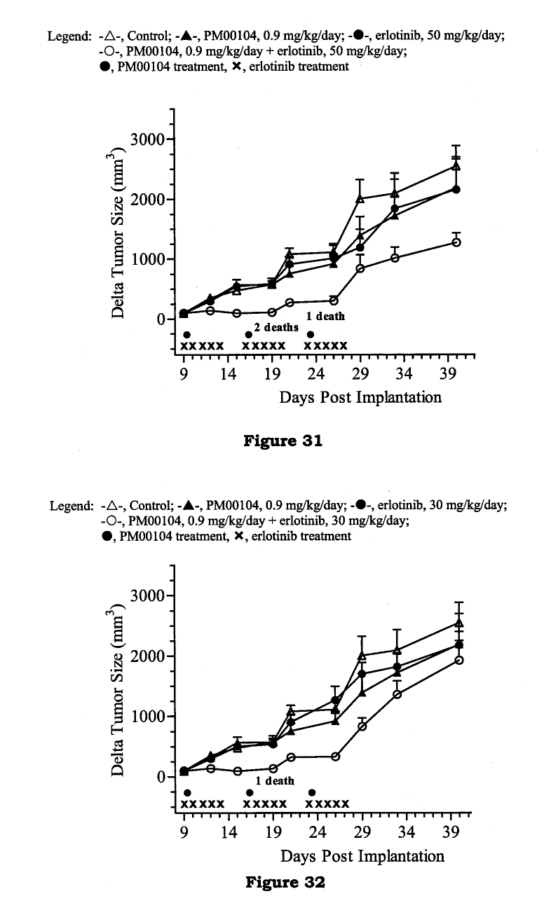

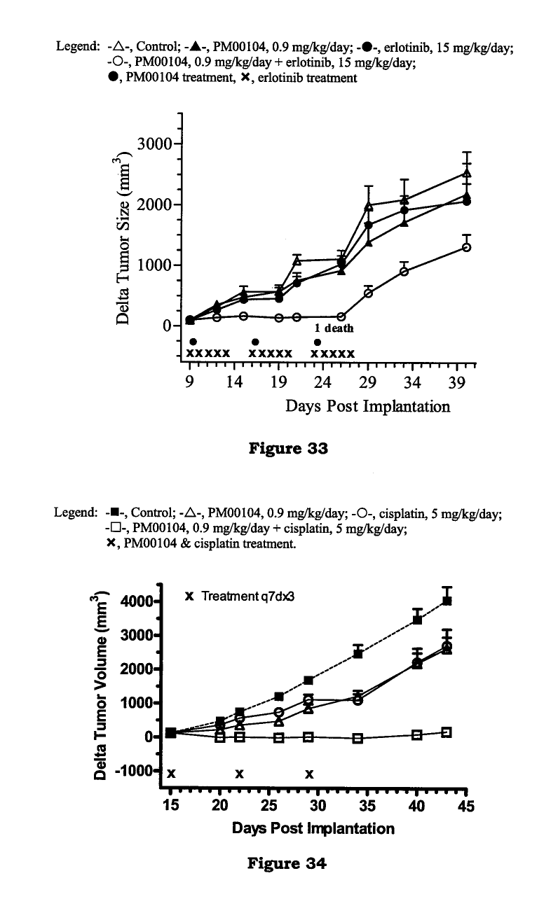

Table 13 reports the % T/C values obtained with each of the treatments and FIG. 31-33 show the tumor volume evaluation (mean±SEM) of BxPC-3 tumors in mice treated with control (vehicle), PM00104, erlotinib, and PM00104 plus erlotinib at different doses.

TABLE 13

| | % T/C on day | | | | | | | | |
|---|---|---|---|---|---|---|---|---|---|
| Group | 9 | 12 | 15 | 19 | 21 | 26 | 29 | 33 | 40 |
| G1 | — | — | — | — | — | — | — | — | — |
| (Control | | | | | | | | | |
| group) | | | | | | | | | |
| G2 | 103.8 | 90.0 | 120.0 | 98.6 | 69.9 | 82.6 | 69.4 | 82.2 | 85.8 |
| G3 | 102.5 | 84.9 | 114.2 | 102.6 | 84.5 | 91.0 | 59.3 | 87.9 | 84.6 |
| G4 | 104.2 | 84.9 | 105.8 | 93.5 | 83.7 | 114.2 | 84.8 | 87.0 | 85.5 |
| G5 | 99.5 | 75.9 | 92.2 | 79.0 | 66.2 | 92.5 | 83.8 | 92.1 | 81.2 |
| G6 | 105.8 | 39.5 | 19.9 | 18.8 | 25.4 | 27.0 | 42.0 | 48.6 | 49.9 |
| G7 | 103.0 | 38.7 | 19.6 | 23.1 | 29.9 | 29.7 | 41.5 | 65.0 | 75.6 |
| G8 | 106.1 | 40.2 | 34.6 | 23.7 | 14.0 | 14.3 | 27.4 | 43.6 | 51.7 |

Table 14 shows the % of tumor growth inhibition of PM00104 and erlotinib administered as single agents and in combination at a dose of 0.9 mg/kg/day of PM00104 and 50 mg/kg/day of erlotinib. Additionally, the potentiation and the degree of additivity of the combination of PM00104 with erlotinib at said doses are provided.

TABLE 14

| | % Inhibition | | | Expected | Actual | | Degree of |
|---|---|---|---|---|---|---|---|
| Day | G2 | G3 | G6 | Response | Response | Potentiation | Response |
| 9 | −3.8 | −2.5 | −5.8 | −6.3 | 0.5 | no | — |
| 12 | 10.0 | 15.1 | 60.5 | 25.1 | 35.4 | yes | Greater than additive |
| 15 | −20.0 | −14.2 | 80.1 | −34.2 | 114.3 | yes | Greater than additive |
| 19 | 1.4 | −2.6 | 81.2 | −1.2 | 82.4 | yes | Greater than additive |
| 21 | 30.1 | 15.5 | 74.6 | 45.6 | 29.0 | yes | Greater than additive |
| 26 | 17.4 | 9.0 | 73.0 | 26.4 | 46.6 | yes | Greater than additive |
| 29 | 30.6 | 40.7 | 58.0 | 71.3 | −13.3 | yes | Less than additive |
| 33 | 17.8 | 12.1 | 51.4 | 29.9 | 21.5 | yes | Greater than additive |
| 40 | 14.2 | 15.4 | 50.1 | 29.6 | 20.5 | yes | Greater than additive |

Table 15 shows the % of tumor growth inhibition of PM00104 and erlotinib administered as single agents and in combination at a dose of 0.9 mg/kg/day of PM00104 and 30 mg/kg/day of erlotinib. Additionally, the potentiation and the degree of additivity of the combination of PM00104 with erlotinib at said doses are provided.

TABLE 15

| | % Inhibition | | | Expected | Actual | | Degree of |
|---|---|---|---|---|---|---|---|
| Day | G2 | G4 | G7 | Response | Response | Potentiation | Response |
| 9 | −3.8 | −4.2 | −3.0 | −8.0 | 5.0 | no | — |
| 12 | 10.0 | 15.1 | 61.3 | 25.1 | 36.2 | yes | Greater than additive |
| 15 | −20.0 | −5.8 | 80.4 | −25.8 | 106.2 | yes | Greater than additive |
| 19 | 1.4 | 6.5 | 76.9 | 7.9 | 69.0 | yes | Greater than additive |
| 21 | 30.1 | 16.3 | 70.1 | 46.4 | 23.7 | yes | Greater than additive |
| 26 | 17.4 | −14.2 | 70.3 | 3.2 | 67.1 | yes | Greater than additive |
| 29 | 30.6 | 15.2 | 58.5 | 45.8 | 12.7 | yes | Greater than additive |
| 33 | 17.8 | 13.0 | 35.0 | 30.8 | 4.2 | yes | Greater than additive |
| 40 | 14.2 | 14.5 | 24.4 | 28.7 | −4.3 | yes | Additive |

Table 16 shows the % of tumor growth inhibition of PM00104 and erlotinib administered as single agents and in combination at a dose of 0.9 mg/kg/day of PM00104 and 15 mg/kg/day of erlotinib. Additionally, the potentiation and the degree of additivity of the combination of PM00104 with erlotinib at said doses are provided.

TABLE 16

| | % Inhibition | | | Expected | Actual | | Degree of |
|---|---|---|---|---|---|---|---|
| Day | G2 | G5 | G8 | Response | Response | Potentiation | Response |
| 9 | −3.8 | 0.5 | −6.1 | −3.3 | −2.8 | no | — |
| 12 | 10.0 | 24.1 | 59.8 | 34.1 | 25.7 | yes | Greater than additive |
| 15 | −20.0 | 7.8 | 65.4 | −12.2 | 77.6 | yes | Greater than additive |
| 19 | 1.4 | 21.0 | 76.3 | 22.4 | 53.9 | yes | Greater than additive |
| 21 | 30.1 | 33.8 | 86 | 63.9 | 22.1 | yes | Greater than additive |
| 26 | 17.4 | 7.5 | 85.7 | 24.9 | 60.8 | yes | Greater than additive |
| 29 | 30.6 | 16.2 | 72.6 | 46.8 | 25.8 | yes | Greater than additive |
| 33 | 17.8 | 7.9 | 56.4 | 25.7 | 30.7 | yes | Greater than additive |
| 40 | 14.2 | 18.8 | 48.3 | 33.0 | 15.3 | yes | Greater than additive |

According to this assay it was found that the combination of PM00104 with erlotinib, at the three doses tested, provided a greater than additive potentiation of the antitumor activity.

### Example 7

In vivo studies to determine the effect of PM00104 in combination with cisplatin, paclitaxel, and gemcitabine in human bladder tumor xenografts.

The aim of these studies was to evaluate the ability of PM00104 to potentiate the antitumor activity of cisplatin, paclitaxel, and gemcitabine by using a xenograft model of human bladder cancer.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation with a tumor cell suspension. The Vehicle Control group contained 15 mice and the treated groups had each 10 mice/group.

The tumor model used in these studies was UM-UC-3 cell line, which was obtained from the ATCC (Manassas, Va.).

UM-UC-3 cells were grown in minimum essential medium (Eagle's) supplemented with 10% FBS, 1.5 g/L sodium bicarbonate, 0.1 mM non-essential amino acids, 1 mM sodium pyruvate, and 2 mM L-glutamine. Each animal was implanted SC on the right flank, using a trochar and 1 cc syringe, with 5×10⁶ UM-UC-3 cells, from in vitro passage 17, in a 0.2 mL suspension of 50% Matrigel and 50% serum free medium, without antibiotics. Bacterial cultures were performed on aliquots of the cells prepared for implantation. All cultures were negative for bacterial contamination at both 24 and 48 hours post-implant.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 15.

PM00104 was provided in the form of vials of lyophilized PM00104 powder which was reconstituted with water for injection. Gemcitabine was provided in the form of a solid white powder containing Gemcitabine HCl, which was reconstituted in 0.9% saline. Cisplatin and paclitaxel were provided as solutions which were further diluted with 0.9% saline.

Study groups and treatment regimens are listed in table 17.

TABLE 17

| Group | Dose | Route | Schedule | Test material |
|---|---|---|---|---|
| G1 | 10 ml/kg/day | IV | A | 0.18% Placebo |
| (Control group) | | | | in Saline |
| G2 | 0.90 mg/kg/day | IV | A | PM00104 |
| G3 | 5 mg/kg/day | IP | A | Cisplatin |
| G4 | 180 mg/kg/day | IP | A | Gemcitabine |
| G5 | 15 mg/kg/day | IP | B | Paclitaxel |
| G6 | 0.90 mg/kg/day | IV | A | PM00104 |
| | 5 mg/kg/day | IP | A | Cisplatin |
| G7 | 0.90 mg/kg/day | IV | A | PM00104 |
| | 180 mg/kg/day | IP | A | Gemcitabine |
| G8 | 0.90 mg/kg/day | IV | A | PM00104 |
| | 15 mg/kg/day | IP | B | Paclitaxel |

A: DPI 15, 22, and 29;
B: DPI 15, 19, and 23
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and cisplatin, PM00104 and gemcitabine or PM00104 and paclitaxel) against cisplatin, gemcitabine or paclitaxel mean tumor weight, respectively, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

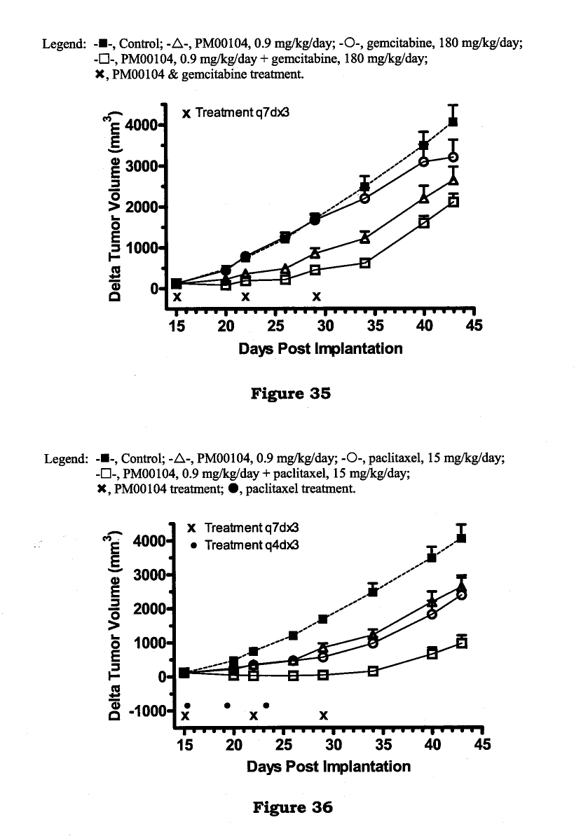

Table 18 reports the % T/C values obtained with each of the treatments and FIG. 34-36 show the tumor volume evaluation (mean±SEM) of UM-UC-3 tumors in mice treated with control (vehicle), PM00104, cisplatin, gemcitabine, paclitaxel, and the corresponding combinations.

TABLE 18

| | % T/C on day | | | | | | | |
|---|---|---|---|---|---|---|---|---|
| Group | 15 | 20 | 22 | 26 | 29 | 34 | 40 | 43 |
| G1 | — | — | — | — | — | — | — | — |
| (Control group) | | | | | | | | |
| G2 | 100.8 | 47.2 | 47.8 | 39.8 | 50.4 | 49.3 | 62.9 | 64.9 |
| G3 | 102.2 | 77.0 | 75.8 | 61.8 | 66.0 | 44.7 | 63.8 | 67.0 |
| G4 | 105.0 | 94.2 | 104.8 | 104.2 | 98.8 | 88.6 | 88.5 | 78.8 |
| G5 | 98.7 | 53.1 | 46.0 | 38.6 | 33.9 | 39.5 | 52.5 | 59.0 |
| G6 | 101.6 | −1.8 | 1.1 | −1.1 | 1.1 | −0.7 | 2.7 | 4.4 |
| G7 | 106.1 | 17.1 | 25.1 | 17.7 | 26.4 | 24.9 | 45.6 | 51.9 |
| G8 | 105.0 | 10.5 | 4.4 | 2.2 | 2.7 | 6.4 | 18.8 | 24.0 |

Table 19 shows the % of tumor growth inhibition of PM00104 and cisplatin administered as single agents and in combination at a dose of 0.9 mg/kg/day of PM00104 and 5 mg/kg/day of cisplatin. Additionally, the potentiation and the degree of additivity of the combination of PM00104 with cisplatin at said doses are provided.

TABLE 19

| | % Inhibition | | | Expected | Actual | | Degree of |
|---|---|---|---|---|---|---|---|
| Day | G2 | G3 | G6 | Response | Response | Potentiation | Response |
| 15 | −0.8 | −2.2 | −1.6 | −3.0 | 1.4 | no | — |
| 20 | 52.8 | 23.0 | 101.8 | 75.8 | 26.0 | yes | Greater than additive |
| 22 | 52.2 | 24.2 | 98.9 | 76.4 | 22.5 | yes | Greater than additive |
| 26 | 60.2 | 38.2 | 101.1 | 98.4 | 2.7 | yes | Greater than additive |
| 29 | 47.4 | 34.0 | 98.9 | 81.4 | 17.5 | yes | Greater than additive |
| 34 | 50.7 | 55.3 | 100.7 | 106.0 | −5.3 | yes | Additive |
| 40 | 37.1 | 36.2 | 97.3 | 73.3 | 24.0 | yes | Greater than additive |
| 43 | 35.1 | 33.0 | 95.6 | 68.1 | 27.5 | yes | Greater than additive |

Table 20 shows the % of tumor growth inhibition of PM00104 and gemcitabine administered as single agents and in combination at a dose of 0.9 mg/kg/day of PM00104 and 180 mg/kg/day of gemcitabine. Additionally, the potentiation and the degree of additivity of the combination of PM00104 with gemcitabine at said doses are provided.

TABLE 20

| | % Inhibition | | | Expected | Actual | | Degree of |
|---|---|---|---|---|---|---|---|
| Day | G2 | G4 | G7 | Response | Response | Potentiation | Response |
| 15 | −0.8 | −5.0 | −6.1 | −5.8 | −0.3 | no | — |
| 20 | 52.8 | 5.8 | 82.9 | 58.6 | 24.3 | yes | Greater than additive |
| 22 | 52.2 | −4.8 | 74.9 | 47.4 | 27.5 | yes | Greater than additive |
| 26 | 60.2 | −4.2 | 82.3 | 56.0 | 26.3 | yes | Greater than additive |
| 29 | 47.4 | 1.2 | 73.6 | 48.6 | 25.0 | yes | Greater than additive |
| 34 | 50.7 | 11.4 | 75.1 | 62.1 | 13.0 | yes | Greater than additive |
| 40 | 37.1 | 11.5 | 54.4 | 48.6 | 5.8 | yes | Additive |
| 43 | 35.1 | 21.2 | 48.1 | 56.3 | −8.2 | yes | Less than additive |

Table 21 shows the % of tumor growth inhibition of PM00104 and paclitaxel administered as single agents and in combination at a dose of 0.9 mg/kg/day of PM00104 and 15 mg/kg/day of paclitaxel. Additionally, the potentiation and the degree of additivity of the combination of PM00104 with paclitaxel at said doses are provided.

TABLE 21

| | % Inhibition | | | Expected | Actual | | Degree of |
|---|---|---|---|---|---|---|---|
| Day | G2 | G5 | G8 | Response | Response | Potentiation | Response |
| 15 | −0.8 | 1.3 | −5.0 | 0.5 | −5.5 | no | — |
| 20 | 52.8 | 46.9 | 89.5 | 99.7 | −10.2 | yes | Less than additive |
| 22 | 52.2 | 54.0 | 95.6 | −106.2 | −10.6 | yes | Less than additive |
| 26 | 60.2 | 61.4 | 97.8 | 121.6 | −23.8 | yes | Less than additive |
| 29 | 47.4 | 64.6 | 97.2 | 112.0 | −14.8 | yes | Less than additive |
| 34 | 50.7 | 60.5 | 93.6 | 111.2 | −17.6 | yes | Less than additive |
| 40 | 37.1 | 47.5 | 81.2 | 84.6 | −3.4 | yes | Additive |
| 43 | 35.1 | 41.0 | 76.0 | 76.1 | −0.1 | yes | Additive |

According to this assay it was found that:

a. The combination of PM00104 and cisplatin resulted in a statistically significant (p<0.001) greater than additive potentiation of antitumor activity.
b. The combination of PM00104 and gemcitabine resulted in a statistically significant (p≦0.001) antitumor activity compared to the control group. At the end of the treatment period (DPI 29) the potentiation was determined to be greater than additive.
c. The combination of PM00104 and paclitaxel resulted in statistically significant potentiation (p<0.001) of antitumor activity when compared to monotherapy controls. This potentiation was determined to be additive at the end of the experiment.

### Example 8

In vivo studies to determine the effect of PM00104 in combination with cisplatin and paclitaxel in human gastric tumor xenografts.

The aim of these studies was to evaluate the ability of PM00104 to potentiate the antitumor activity of cisplatin and paclitaxel by using a xenograft model of human gastric carcinoma.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation with a tumor cell suspension. The Vehicle Control group contained 11 mice, groups 2-4 contained 7 mice and the rest of groups contained 8 mice.

The tumor model used in these studies was Hs746T cell line, which was obtained from the ATCC (Manassas, Va.).

Hs746T cells were grown in DMEM supplemented with 10% FBS, 1.5 g/L sodium bicarbonate, 4.5 g/L glucose, and 4 mM L-glutamine. Each animal was implanted SC on the right flank, using a trochar, with 5×10⁶ Hs746T cells, from in vitro passage 18, in a 0.2 mL suspension of 50% Matrigel and 50% serum free medium, without antibiotics. Bacterial cultures were performed on aliquots of the cells prepared for implantation. All cultures were negative for bacterial contamination at both 24 and 48 hours post-implant.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 16.

PM00104 was provided in the form of vials of lyophilized PM00104 powder which was reconstituted with water for injection. Cisplatin and paclitaxel were provided as solutions which were further diluted with 0.9% saline.

Study groups and treatment regimens are listed in table 22.

TABLE 22

| Group | Dose | Route | Schedule | Test material |
|---|---|---|---|---|
| G1 | 10 ml/kg/day | IV | A | 0.18% Placebo in |
| (Control group) | | | | Saline |
| G2 | 0.90 mg/kg/day | IV | A | PM00104 |
| G3 | 5 mg/kg/day | IP | B | Cisplatin |
| G4 | 10 mg/kg/day | IP | C | Paclitaxel |
| G5 | 0.90 mg/kg/day | IV | A | PM00104 |
| | 5 mg/kg/day | IP | B | Cisplatin |
| G6 | 0.90 mg/kg/day | IV | A | PM00104 |
| | 10 mg/kg/day | IP | C | Paclitaxel |

A: DPI 16, 23, and 30;
B: DPI 16, 26, and 33;
C: DPI 16, 20, 24
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and cisplatin or PM00104 and paclitaxel) against cisplatin or paclitaxel mean tumor weight, respectively, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

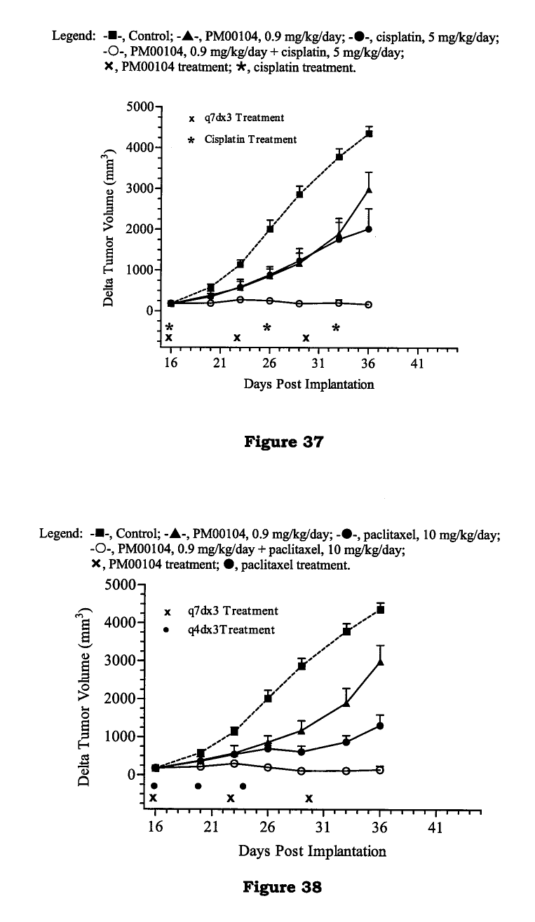

Table 23 reports the % T/C values obtained with each of the treatments and FIG. 37-38 show the tumor volume evaluation (mean±SEM) of Hs746T tumors in mice treated with control (vehicle), PM00104, cisplatin, paclitaxel, and the corresponding combinations.

TABLE 23

| | % T/C on day | | | | | | |
|---|---|---|---|---|---|---|---|
| Group | 16 | 20 | 23 | 26 | 29 | 33 | 36 |
| G1 | — | — | — | — | — | — | — |
| (Control group) | | | | | | | |
| G2 | 98.0 | 65.6 | 50.3 | 42.8 | 40.9 | 50.2 | 68.6 |
| G3 | 96.8 | 60.5 | 51.7 | 44.2 | 43.3 | 46.7 | 46.3 |
| G4 | 96.4 | 63.3 | 47.7 | 34.7 | 21.4 | 23.2 | 30.1 |
| G5 | 102.9 | 32.3 | 24.5 | 12.5 | 6.4 | 5.4 | 3.9 |
| G6 | 103.5 | 37.3 | 26.7 | 10.1 | 3.9 | 3.2 | 3.3 |

Table 24 shows the % of tumor growth inhibition of PM00104 and cisplatin administered as single agents and in combination at a dose of 0.9 mg/kg/day of PM00104 and 5 mg/kg/day of cisplatin. Additionally, the potentiation and the degree of additivity of the combination of PM00104 with cisplatin at said doses are provided.

TABLE 24

| | % Inhibition | | | Expected | Actual | | Degree of |
|---|---|---|---|---|---|---|---|
| Day | G2 | G3 | G5 | Response | Response | Potentiation | Response |
| 16 | 2.0 | 3.2 | −2.9 | 5.2 | −8.1 | no | — |
| 20 | 34.4 | 39.5 | 67.7 | 73.9 | −6.2 | yes | Less than additive |
| 23 | 49.7 | 48.3 | 75.5 | 98.0 | −22.5 | yes | Less than additive |
| 26 | 57.2 | 55.8 | 87.5 | 113.0 | −25.5 | yes | Less than additive |
| 29 | 59.1 | 56.7 | 93.6 | 115.8 | −22.2 | yes | Less than additive |
| 33 | 49.8 | 53.3 | 94.6 | 103.1 | −8.5 | yes | Additive |
| 36 | 31.4 | 53.7 | 96.1 | 85.1 | 11.0 | yes | Greater than additive |

Table 25 shows the % of tumor growth inhibition of PM00104 and paclitaxel administered as single agents and in combination at a dose of 0.9 mg/kg/day of PM00104 and 10 mg/kg/day of paclitaxel. Additionally, the potentiation and the degree of additivity of the combination of PM00104 with paclitaxel at said doses are provided.

TABLE 25

| | % Inhibition | | | Expected | Actual | | Degree of |
|---|---|---|---|---|---|---|---|
| Day | G2 | G4 | G6 | Response | Response | Potentiation | Response |
| 16 | 2.0 | 3.6 | −3.5 | 5.6 | −9.1 | no | — |
| 20 | 34.4 | 36.7 | 62.7 | 71.1 | −8.4 | yes | Less than additive |
| 23 | 49.7 | 52.3 | 73.3 | 102.0 | −28.7 | yes | Less than additive |
| 26 | 57.2 | 65.3 | 89.9 | 122.5 | −32.6 | yes | Less than additive |
| 29 | 59.1 | 78.6 | 96.1 | 137.7 | −41.6 | yes | Less than additive |
| 33 | 49.8 | 76.8 | 96.8 | 126.6 | −29.8 | yes | Less than additive |
| 36 | 31.4 | 69.9 | 96.7 | 101.3 | −4.6 | yes | Additive |

According to this assay it was found that:

a. The combination of PM00104 and cisplatin resulted in a statistically significant (p<0.01) potentiation of antitumor activity over results obtained with either of the single agent control groups, resulting in an almost complete regression of the tumors in the combination group. At the end of the follow-up period, the potentiation was determined to be greater than additive.
b. During the treatment period as well as at the end of the follow-up period, the combination of PM00104 and paclitaxel resulted in a statistically significant (p≦0.01) potentiation of antitumor activity over results obtained with either of the single agent control groups, resulting in an almost complete regression of the tumors.

### Example 9

In vivo studies to determine the effect of PM00104 in combination with fluorouracil (5-FU), irinotecan, and doxorubicin in human gastric tumor xenografts.

The aim of these studies was to evaluate the ability of PM00104 to potentiate the antitumor activity of 5-FU, irinotecan, and doxorubicin by using a xenograft model of human gastric carcinoma.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation with a tumor cell suspension. The Vehicle Control group contained 15 mice and the treated groups had each 10 mice/group.

The tumor model used in these studies was Hs746T cell line, which was obtained from the ATCC (Manassas, Va.). This cell line was grown and implanted to the animals as described in Example 8.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 15.

PM00104 was provided in the form of vials of lyophilized PM00104 powder which was reconstituted with water for injection. 5-FU was provided as a solution which was further diluted with water for injection. Irinotecan was provided in the form a solution containing Irinotecan HCl trihydrate, which was diluted in 0.9% sterile saline.

Doxorubicin was provided in the form of a lyophilized powder, which was reconstituted in 0.9% saline.

Study groups and treatment regimens are listed in table 26.

TABLE 26

| Group | Dose | Route | Schedule | Test material |
|---|---|---|---|---|
| G1 | 10 ml/kg/day | IV | A | 0.18% Placebo |
| (Control group) | | | | in Saline |
| G2 | 0.90 mg/kg/day | IV | A | PM00104 |
| G3 | 50 mg/kg/day | IP | B | 5-FU |
| | 100 mg/kg/day | | C | 5-FU |
| G4 | 20 mg/kg/day | IP | A | Irinotecan |
| G5 | 6 mg/kg/day | IP | D | Doxorubicin |
| G6 | 0.90 mg/kg/day | IV | A | PM00104 |
| | 50 mg/kg/day | IP | B | 5-FU |
| | 100 mg/kg/day | IP | C | 5-FU |
| G7 | 0.90 mg/kg/day | IV | A | PM00104 |
| | 20 mg/kg/day | IP | A | Irinotecan |
| G8 | 0.90 mg/kg/day | IV | A | PM00104 |
| | 6 mg/kg/day | IP | D | Doxorubicin |

A: DPI 15, 22, and 29;
B: DPI 15;
C: DPI 22 and 29;
D: DPI 15, 19, and 23
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and 5-FU, PM00104 and irinotecan or PM00104 and doxorubicin) against 5-FU, irinotecan or doxorubicin mean tumor weight, respectively, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

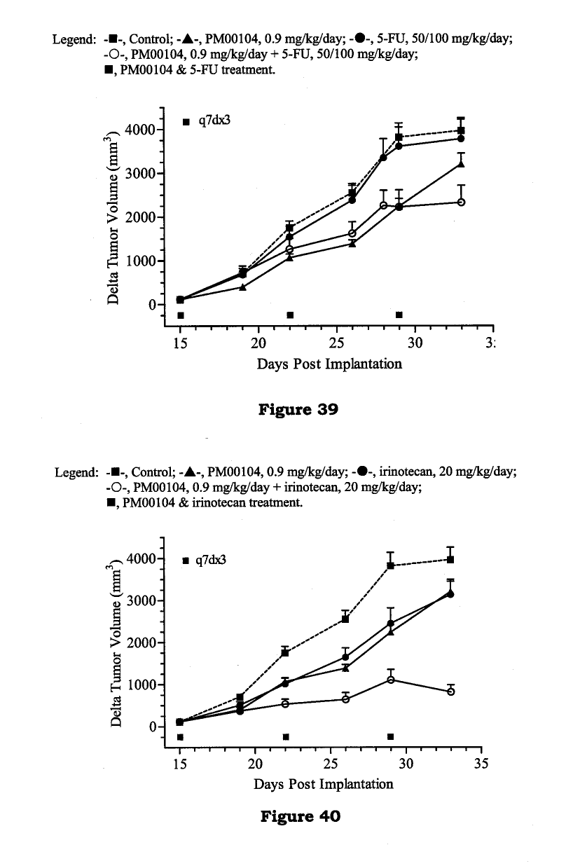

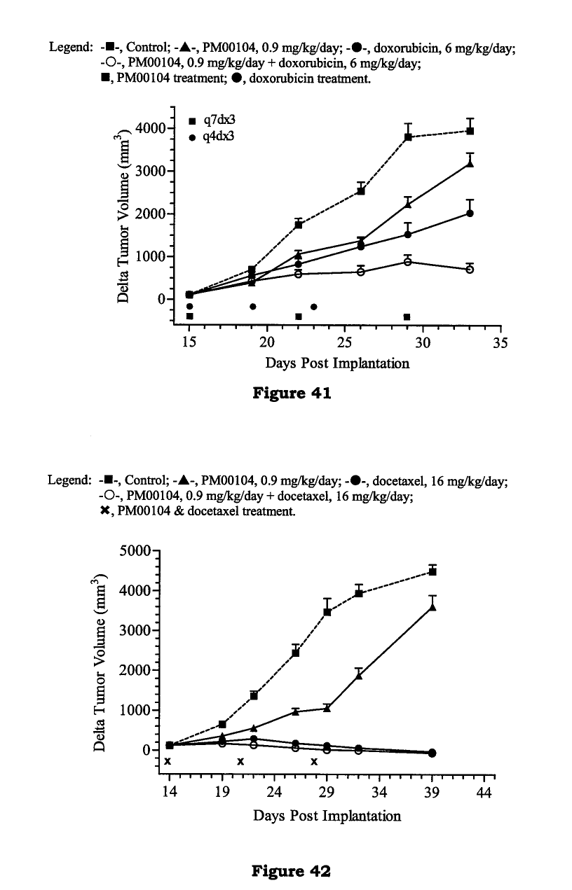

Table 27 reports the % T/C values obtained with each of the treatments and FIG. 39-41 show the tumor volume evaluation (mean±SEM) of Hs746T tumors in mice treated with control (vehicle), PM00104, 5-FU, irinotecan, doxorubicin, and the corresponding combinations.

TABLE 27

| | % T/C on day | | | | | |
|---|---|---|---|---|---|---|
| Group | 15 | 19 | 22 | 26 | 29 | 33 |
| G1 | — | — | — | — | — | — |
| (Control group) | | | | | | |
| G2 | 106.6 | 55.8 | 60.9 | 54.2 | 58.6 | 80.8 |
| G3 | 102.3 | 96.4 | 88.0 | 93.4 | 94.5 | 95.3 |
| G4 | 103.4 | 72.3 | 57.9 | 64.6 | 64.2 | 79.0 |
| G5 | 104.2 | 79.8 | 47.4 | 48.9 | 40.2 | 51.4 |
| G6 | 104.4 | 103.4 | 72.1 | 63.6 | 58.3 | 58.6 |
| G7 | 105.9 | 52.7 | 30.4 | 25.1 | 28.9 | 20.5 |
| G8 | 100.3 | 61.2 | 34.1 | 25.5 | 23.4 | 18.1 |

Table 28 shows the % of tumor growth inhibition of PM00104 and 5-FU administered as single agents and in combination at a dose of 0.9 mg/kg/day of PM00104 and 50 mg/kg/day of 5-FU at DPI 15 and 100 mg/kg/day of 5-FU at DPI 22 & 29. Additionally, the potentiation and the degree of additivity of the combination of PM00104 with 5-FU at said doses are provided.

TABLE 28

| | % Inhibition | | | Expected | Actual | | Degree of |
|---|---|---|---|---|---|---|---|
| Day | G2 | G3 | G6 | Response | Response | Potentiation | Response |
| 15 | −6.6 | −2.3 | −4.4 | −8.9 | 4.5 | no | — |
| 19 | 44.2 | 3.6 | −3.4 | 47.8 | −51.2 | no | — |
| 22 | 39.1 | 12.0 | 27.9 | 51.1 | −23.2 | no | — |
| 26 | 45.8 | 6.6 | 36.4 | 52.4 | −16.0 | no | — |
| 29 | 41.4 | 5.5 | 41.7 | 46.9 | −5.2 | no | — |
| 33 | 19.2 | 4.7 | 41.4 | 23.9 | 17.5 | yes | Greater than additive |

Table 29 shows the % of tumor growth inhibition of PM00104 and irinotecan administered as single agents and in combination at a dose of 0.9 mg/kg/day of PM00104 and 20 mg/kg/day of irinotecan. Additionally, the potentiation and the degree of additivity of the combination of PM00104 with irinotecan at said doses are provided.

TABLE 29

| | % Inhibition | | | Expected | Actual | | Degree of |
|---|---|---|---|---|---|---|---|
| Day | G2 | G4 | G7 | Response | Response | Potentiation | Response |
| 15 | −6.6 | −3.4 | −5.9 | −10.0 | 4.1 | no | — |
| 19 | 44.2 | 27.7 | 47.3 | 71.9 | −24.6 | yes | Less than additive |
| 22 | 39.1 | 42.1 | 69.6 | 81.2 | −11.6 | yes | Less than additive |
| 26 | 45.8 | 35.4 | 74.9 | 81.2 | −6.3 | yes | Additive |
| 29 | 41.4 | 35.8 | 71.1 | 77.2 | −6.1 | yes | Additive |
| 33 | 19.2 | 21.0 | 79.5 | 40.2 | 39.3 | yes | Greater than additive |

Table 30 shows the % of tumor growth inhibition of PM00104 and doxorubicin administered as single agents and in combination at a dose of 0.9 mg/kg/day of PM00104 and 6 mg/kg/day of doxorubicin. Additionally, the potentiation and the degree of additivity of the combination of PM00104 with doxorubicin at said doses are provided.

TABLE 30

| | % Inhibition | | | Expected | Actual | | Degree of |
|---|---|---|---|---|---|---|---|
| Day | G2 | G5 | G8 | Response | Response | Potentiation | Response |
| 15 | −6.6 | −4.2 | −0.3 | −10.8 | 10.5 | no | — |
| 19 | 44.2 | 20.2 | 38.8 | 64.4 | −25.6 | no | — |
| 22 | 39.1 | 52.6 | 65.9 | 91.7 | −25.8 | yes | Less than additive |
| 26 | 45.8 | 51.1 | 74.5 | 96.9 | −22.4 | yes | Less than additive |
| 29 | 41.4 | 59.8 | 76.6 | 101.2 | −24.6 | yes | Less than additive |
| 33 | 19.2 | 48.6 | 81.9 | 67.8 | 14.1 | yes | Greater than additive |

According to this assay it was found that:

a. The combination of PM00104 and 5-FU resulted in additive potentiation of antitumor activity during the treatment period and was determined greater than additive at the end of the observation period.
b. The combination of PM00104 and irinotecan resulted in a highly statistically significant (p<0.001) potentiation of antitumor activity over results obtained with either of the single agent control groups, with the potentiation being graded as greater than additive at the end of the experiment.
c. The combination of PM00104 and doxorubicin resulted in a statistically significant (p<0.01) potentiation of antitumor activity over results obtained with either of the single agent control groups, with the potentiation being graded as greater than additive at the end of the experiment.

### Example 10

In vivo studies to determine the effect of PM00104 in combination with docetaxel and oxaliplatin in human gastric tumor xenografts.

The aim of these studies was to evaluate the ability of PM00104 to potentiate the antitumor activity of docetaxel and oxaliplatin by using a xenograft model of human gastric carcinoma.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation with a tumor cell suspension. The Vehicle Control group contained 15 mice and the treated groups had each 10 mice/group.

The tumor model used in these studies was Hs746T cell line, which was obtained from the ATCC (Manassas, Va.). This cell line was grown and implanted to the animals as described in Example 8.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 14.

PM00104 was provided in the form of vials of lyophilized PM00104 powder which was reconstituted with water for injection. Docetaxel was provided as a concentrated for dilution which was further diluted with 13% ethanol in water for injection (wfi). Oxaliplatin was provided as a solution which was further diluted with 5% Dextrose injection, USP.

Study groups and treatment regimens are listed in table 31.

TABLE 31

| Group | Dose | Route | Schedule | Test material |
|---|---|---|---|---|
| G1 | 10 ml/kg/day | IV | A | 0.18% Placebo in |
| (Control group) | 10 ml/kg/day | IP | A | Saline |
| | | | | 0.52% Ethanol in |
| | | | | wfi |
| G2 | 0.90 mg/kg/day | IV | A | PM00104 |
| G3 | 16 mg/kg/day | IP | A | Docetaxel |
| G4 | 8 mg/kg/day | IP | A | Docetaxel |
| G5 | 8 mg/kg/day | IP | A | Oxaliplatin |
| G6 | 4 mg/kg/day | IP | A | Oxaliplatin |
| G7 | 0.90 mg/kg/day | IV | A | PM00104 |
| | 16 mg/kg/day | IP | A | Docetaxel |
| G8 | 0.90 mg/kg/day | IV | A | PM00104 |
| | 8 mg/kg/day | IP | A | Docetaxel |
| G9 | 0.90 mg/kg/day | IV | A | PM00104 |
| | 8 mg/kg/day | IP | A | Oxaliplatin |
| G10 | 0.90 mg/kg/day | IV | A | PM00104 |
| | 4 mg/kg/day | IP | A | Oxaliplatin |

A: DPI 14, 21, and 28;
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and docetaxel or PM00104 and oxaliplatin) against docetaxel or oxaliplatin mean tumor weight, respectively, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

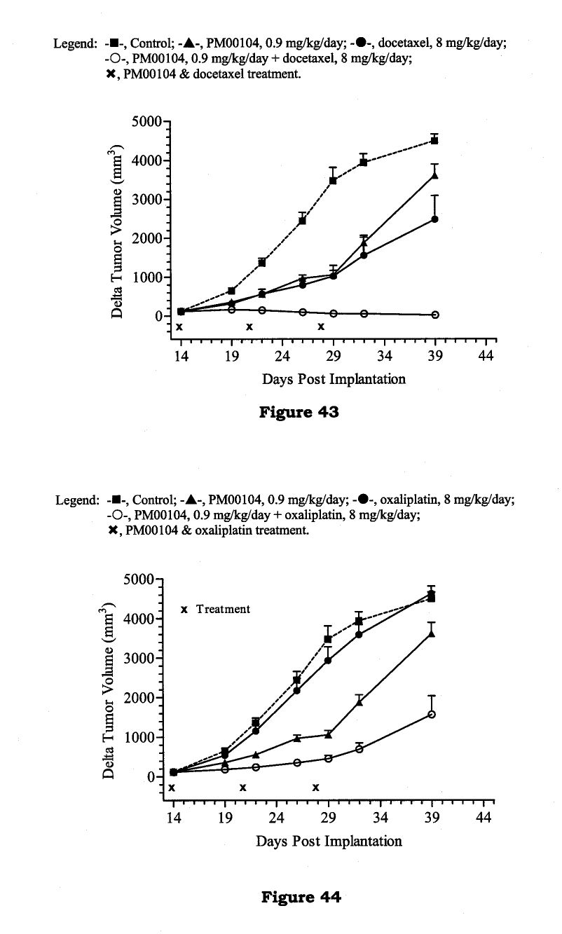

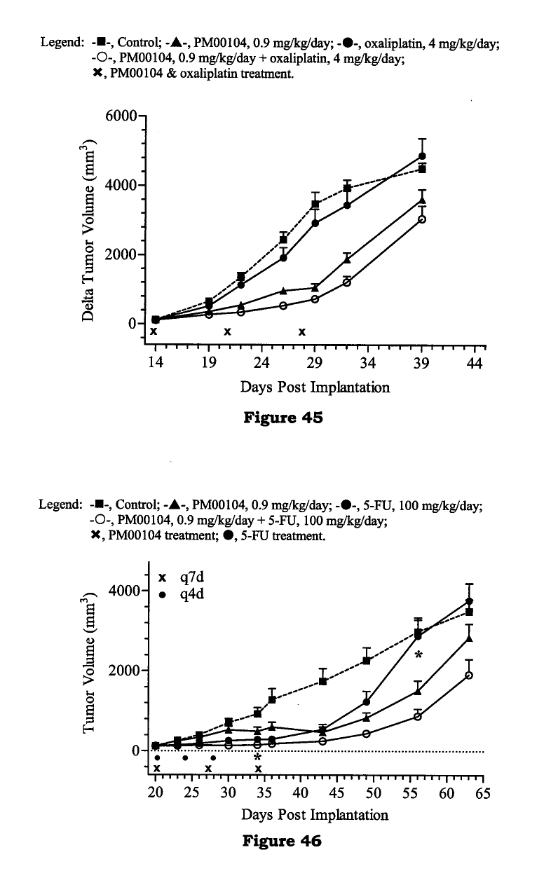

Table 32 reports the % T/C values obtained with each of the treatments and FIG. 42-45 show the tumor volume evaluation (mean±SEM) of Hs746T tumors in mice treated with control (vehicle), PM00104, docetaxel, oxaliplatin, and the corresponding combinations.

TABLE 32

| | % T/C on day | | | | | | |
|---|---|---|---|---|---|---|---|
| Group | 14 | 19 | 22 | 26 | 29 | 32 | 39 |
| G1 | — | — | — | — | — | — | — |
| (Control group) | | | | | | | |
| G2 | 99.9 | 54.9 | 40.8 | 39.5 | 30.4 | 47.6 | 80.3 |
| G3 | 100.3 | 33.6 | 21.3 | 7.2 | 3.5 | 1.5 | −0.6 |
| G4 | 100.8 | 49.0 | 41.6 | 32.5 | 29.4 | 39.4 | 55.1 |
| G5 | 99.6 | 83.5 | 84.9 | 89.1 | 84.5 | 91.2 | 102.9 |
| G6 | 99.9 | 79.9 | 83.8 | 78.6 | 84.2 | 87.5 | 108.4 |
| G7 | 105.2 | 26.1 | 9.5 | 2.5 | 0.3 | 0.0 | −1.3 |
| G8 | 99.2 | 25.3 | 10.7 | 3.9 | 1.5 | 1.3 | 0.4 |
| G9 | 100.8 | 28.3 | 17.5 | 14.3 | 13.0 | 17.5 | 34.9 |
| G10 | 98.4 | 41.8 | 25.5 | 22.0 | 20.9 | 30.9 | 67.8 |

Table 33 shows the % of tumor growth inhibition of PM00104 and docetaxel administered as single agents and in combination at a dose of 0.9 mg/kg/day of PM00104 and 16 mg/kg/day of docetaxel. Additionally, the potentiation and the degree of additivity of the combination of PM00104 with docetaxel at said doses are provided.

TABLE 33

| | % Inhibition | | | Expected | | | Degree |
|---|---|---|---|---|---|---|---|
| Day | G2 | G3 | G7 | Response | Actual Response | Potentiation | of Response |
| 14 | 0.1 | −0.3 | −5.2 | −0.2 | −5.0 | no | — |
| 19 | 45.1 | 66.4 | 73.9 | 111.5 | −37.6 | yes | Less than additive |
| 22 | 59.2 | 78.7 | 90.5 | 137.9 | −47.4 | yes | Less than additive |
| 26 | 60.5 | 92.8 | 97.5 | 153.3 | −55.8 | yes | Less than additive |
| 29 | 69.6 | 96.5 | 99.7 | 166.1 | −66.4 | yes | Less than additive |
| 32 | 52.4 | 98.5 | 100.0 | 150.9 | −50.9 | yes | Less than additive |
| 39 | 19.7 | 100.6 | 101.3 | 120.3 | −19.0 | yes | Less than additive |

Table 34 shows the % of tumor growth inhibition of PM00104 and docetaxel administered as single agents and in combination at a dose of 0.9 mg/kg/day of PM00104 and 8 mg/kg/day of docetaxel. Additionally, the potentiation and the degree of additivity of the combination of PM00104 with docetaxel at said doses are provided.

TABLE 34

| | % Inhibition | | | Expected | Actual | | Degree of |
|---|---|---|---|---|---|---|---|
| Day | G2 | G4 | G8 | Response | Response | Potentiation | Response |
| 14 | 0.1 | −0.8 | 0.8 | −0.7 | 1.5 | no | — |
| 19 | 45.1 | 51.0 | 74.7 | 96.1 | −21.4 | yes | Less than additive |
| 22 | 59.2 | 58.4 | 89.3 | 117.6 | −28.3 | yes | Less than additive |
| 26 | 60.5 | 67.5 | 96.1 | 128.0 | −31.9 | yes | Less than additive |
| 29 | 69.6 | 70.6 | 98.5 | 140.2 | −41.7 | yes | Less than additive |
| 32 | 52.4 | 60.6 | 98.7 | 113.0 | −14.3 | yes | Less than additive |
| 39 | 19.7 | 44.9 | 99.6 | 64.6 | 35.0 | yes | Greater than additive |

Table 35 shows the % of tumor growth inhibition of PM00104 and oxaliplatin administered as single agents and in combination at a dose of 0.9 mg/kg/day of PM00104 and 8 mg/kg/day of oxaliplatin. Additionally, the potentiation and the degree of additivity of the combination of PM00104 with oxaliplatin at said doses are provided.

TABLE 35

| | % Inhibition | | | Expected | Actual | | Degree of |
|---|---|---|---|---|---|---|---|
| Day | G2 | G5 | G9 | Response | Response | Potentiation | Response |
| 14 | 0.1 | 0.4 | −0.8 | 0.5 | −1.3 | no | — |
| 19 | 45.1 | 16.5 | 71.7 | 61.6 | 10.1 | yes | Greater than additive |
| 22 | 59.2 | 15.1 | 82.5 | 74.3 | 8.2 | yes | Greater than additive |
| 26 | 60.5 | 10.9 | 85.7 | 71.4 | 14.3 | yes | Greater than additive |
| 29 | 69.6 | 15.5 | 87.0 | 85.1 | 1.9 | yes | Greater than additive |
| 32 | 52.4 | 8.8 | 84.9 | 61.2 | 23.7 | yes | Greater than additive |
| 39 | 19.7 | −2.9 | 65.1 | 16.8 | 48.3 | yes | Greater than additive |

Table 36 shows the % of tumor growth inhibition of PM00104 and oxaliplatin administered as single agents and in combination at a dose of 0.9 mg/kg/day of PM00104 and 4 mg/kg/day of oxaliplatin. Additionally, the potentiation and the degree of additivity of the combination of PM00104 with oxaliplatin at said doses are provided.

TABLE 36

| | % Inhibition | | | Expected | Actual | | Degree of |
|---|---|---|---|---|---|---|---|
| Day | G2 | G6 | G10 | Response | Response | Potentiation | Response |
| 14 | 0.1 | 0.1 | 1.6 | 0.2 | 1.4 | no | — |
| 19 | 45.1 | 20.1 | 58.2 | 65.2 | −7.0 | yes | Less than additive |
| 22 | 59.2 | 16.2 | 74.5 | 75.4 | −0.9 | yes | Less than additive |
| 26 | 60.5 | 21.4 | 78.0 | 81.9 | −3.9 | yes | Less than additive |
| 29 | 69.6 | 15.8 | 79.1 | 85.4 | −6.3 | yes | Less than additive |
| 32 | 52.4 | 12.5 | 69.1 | 64.9 | 4.2 | yes | Greater than additive |
| 39 | 19.7 | −8.4 | 32.2 | 11.3 | 20.9 | yes | Greater than additive |

According to this assay it was found that:

a. The combination of PM00104 and docetaxel resulted in a statistically significant (p<0.01) potentiation of antitumor activity over results obtained with PM00104 as a single agent control group but not with docetaxel as a single agent control group, with the potentiation being graded as less than additive at the end of the experiment in the case of 16 mg/kg/day of docetaxel and greater than additive at the end of the experiment in the case of 8 mg/kg/day of docetaxel.
b. The combination of PM00104 and oxaliplatin resulted in a statistically significant (p<0.001) potentiation of antitumor activity over results obtained with either of the single agent control groups, with the potentiation being graded as greater than additive at the end of the experiment.

### Example 11

In vivo studies to determine the effect of PM00104 in combination with 5-fluorouracil (5-FU) in human gastric tumor xenografts.

The aim of these studies was to evaluate the ability of PM00104 to potentiate the antitumor activity of 5-FU by using a xenograft model of human gastric carcinoma.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation with tumor fragments. The Vehicle Control group contained 15 mice and the treated groups had each 10 mice/group.

The tumor model used in these studies was MRI-H-254 cell line, which was obtained from the DCT Tumor Bank.

MRI-H-254 fragments were removed from donor animals and tissue was debrided of membrane and any hemorrhagic and necrotic areas and 3-4 mm³ fragments, from in vivo passage 5, were implanted SC on the right flank of each animal, using a 13G trochar. Bacterial culture was taken on cells used to implant the study. All cultures were negative for bacterial contamination at both 24 and 48 hours post-implant.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 20.

PM00104 was provided in the form of vials of lyophilized PM00104 powder which was reconstituted with water for injection. 5-FU was provided in the form of injection vials which was diluted with 0.9% sterile Saline.

Study groups and treatment regimens are listed in table 37.

TABLE 37

| Group | Dose | Route | Schedule | Test material |
|---|---|---|---|---|
| G1 | 10 ml/kg/day | IV | A | 0.18% Placebo |
| (Control group) | | IP | B | in Saline |
| | | | | 0.9% Saline |
| G2 | 0.90 mg/kg/day | IV | A | PM00104 |
| G3 | 100 mg/kg/day | IP | A | 5-FU |
| G4 | 50 mg/kg/day | IP | A | 5-FU |
| G5 | 0.90 mg/kg/day | IV | A | PM00104 |
| | 100 mg/kg/day | IP | A | 5-FU |
| G6 | 0.90 mg/kg/day | IV | A | PM00104 |
| | 50 mg/kg/day | IP | A | 5-FU |

A: DPI 20, 27, and 34;
B: DPI 20, 24, and 28
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and 5-FU) against 5-FU mean tumor weight, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

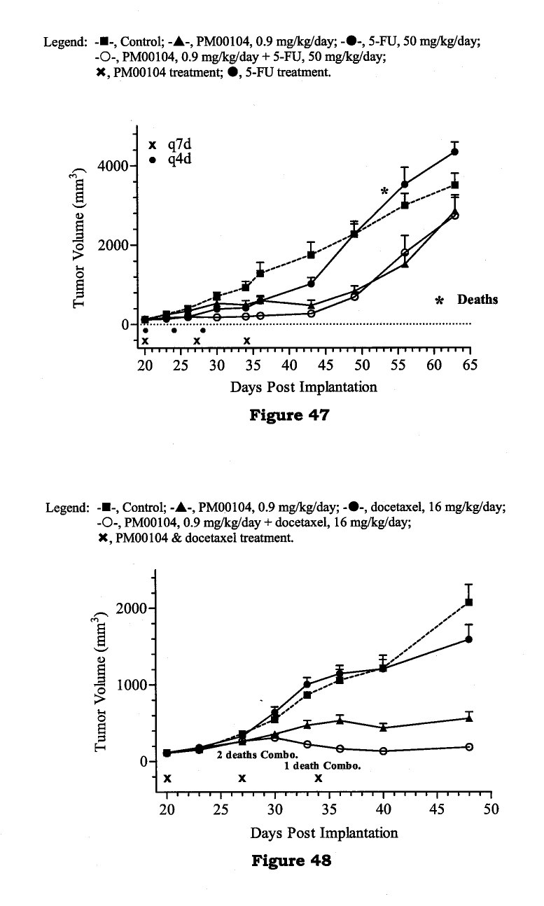

Table 38 reports the % T/C values obtained with each of the treatments and FIG. 46-47 show the tumor volume evaluation (mean±SEM) of MRI-H-254 tumors in mice treated with control (vehicle), PM00104, 5-FU, and the corresponding combinations.

TABLE 38

| | % T/C on day | | | | | | | | | |
|---|---|---|---|---|---|---|---|---|---|---|
| Group | 20 | 23 | 26 | 30 | 34 | 36 | 43 | 49 | 56 | 63 |
| G1 (Control group) | — | — | — | — | — | — | — | — | — | — |
| G2 | 98.7 | 95.9 | 83.7 | 75.3 | 53.1 | 46.5 | 26.9 | 36.7 | 50.6 | 81.3 |
| G3 | 101.8 | 55.2 | 47.1 | 36.3 | 31.6 | 23.0 | 30.9 | 54.6 | 96.3 | 107.8 |
| G4 | 96.0 | 52.3 | 47.9 | 55.3 | 45.0 | 45.7 | 58.1 | 100.0 | 117.6 | 123.9 |
| G5 | 99.0 | 47.8 | 34.8 | 19.4 | 16.1 | 14.1 | 14.2 | 19.3 | 29.1 | 54.8 |
| G6 | 100.4 | 65.0 | 46.5 | 25.3 | 21.2 | 16.8 | 15.3 | 30.2 | 60.2 | 78.0 |

Table 39 shows the % of tumor growth inhibition of PM00104 and 5-FU administered as single agents and in combination at a dose of 0.9 mg/kg/day of PM00104 and 100 mg/kg/day of 5-FU. Additionally, the potentiation and the degree of additivity of the combination of PM00104 with 5-FU at said doses are provided.

TABLE 39

| | % Inhibition | | | Expected | Actual | | Degree of |
|---|---|---|---|---|---|---|---|
| Day | G2 | G3 | G5 | Response | Response | Potentiation | Response |
| 20 | 1.3 | −1.8 | 1.0 | −0.5 | 1.5 | no | — |
| 23 | 4.1 | 44.8 | 52.2 | 48.9 | 3.3 | yes | Additive |
| 26 | 16.3 | 52.9 | 65.2 | 69.2 | −4.0 | yes | Less than additive |
| 30 | 24.7 | 63.7 | 80.6 | 88.4 | −7.8 | yes | Less than additive |
| 34 | 46.9 | 68.4 | 83.9 | 115.3 | −31.4 | yes | Less than additive |
| 36 | 53.5 | 77.0 | 85.9 | 130.5 | −44.6 | yes | Less than additive |
| 43 | 73.1 | 69.1 | 85.8 | 142.2 | −56.4 | yes | Less than additive |
| 49 | 63.3 | 45.4 | 80.7 | 108.7 | −28.0 | yes | Less than additive |
| 56 | 49.4 | 3.7 | 70.9 | 53.1 | 17.8 | yes | Greater than additive |
| 63 | 18.7 | −7.8 | 45.2 | 10.9 | 34.3 | yes | Greater than additive |

Table 40 shows the % of tumor growth inhibition of PM00104 and 5-FU administered as single agents and in combination at a dose of 0.9 mg/kg/day of PM00104 and 50 mg/kg/day of 5-FU. Additionally, the potentiation and the degree of additivity of the combination of PM00104 with 5-FU at said doses are provided.

TABLE 40

| | % Inhibition | | | Expected | Actual | | Degree of |
|---|---|---|---|---|---|---|---|
| Day | G2 | G4 | G6 | Response | Response | Potentiation | Response |
| 20 | 1.3 | 4.0 | −0.4 | 5.3 | −5.7 | no | — |
| 23 | 4.1 | 47.7 | 35.0 | 51.8 | −16.8 | no | — |
| 26 | 16.3 | 52.1 | 53.5 | 68.4 | −14.9 | yes | Less than additive |
| 30 | 24.7 | 44.7 | 74.7 | 69.4 | 5.3 | yes | Additive |
| 34 | 46.9 | 55.0 | 78.8 | 101.9 | −23.1 | yes | Less than additive |
| 36 | 53.5 | 54.3 | 83.2 | 107.8 | −24.6 | yes | Less than additive |
| 43 | 73.1 | 41.9 | 84.7 | 115.0 | −30.3 | yes | Less than additive |
| 49 | 63.3 | 0.0 | 69.8 | 63.3 | 6.5 | yes | Additive |
| 56 | 49.4 | −17.6 | 39.8 | 31.8 | 8.0 | no | — |
| 63 | 18.7 | −23.9 | 22.0 | −5.2 | 27.2 | yes | Greater than additive |

According to this assay it was found that the combination of PM00104 and 5-FU resulted in a statistically significant (p<0.01) potentiation of antitumor activity over results obtained with either of the single agent control groups, with the potentiation being graded as greater than additive at the end of the experiment.

### Example 12

In vivo studies to determine the effect of PM00104 in combination with docetaxel and oxaliplatin in human gastric tumor xenografts.

The aim of these studies was to evaluate the ability of PM00104 to potentiate the antitumor activity of docetaxel and oxaliplatin by using a xenograft model of human gastric carcinoma.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation with tumor fragments. The Vehicle Control group contained 15 mice and the treated groups had each 10 mice/group.

The tumor model used in these studies was MRI-H-254 cell line, which was obtained from the DCT Tumor Bank. This cell line was grown and implanted to the animals as described in Example 11.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 20.

PM00104 was provided in the form of vials of lyophilized PM00104 powder which was reconstituted with water for injection. Docetaxel was provided as a concentrated for dilution which was further diluted with 13% ethanol in water for injection. Oxaliplatin was provided as a solution which was further diluted with 5% Dextrose injection, USP.

Study groups and treatment regimens are listed in table 41.

TABLE 41

| Group | Dose | Route | Schedule | Test material |
|---|---|---|---|---|
| G1 | 10 ml/kg/day | IV | A | 0.18% Placebo in Saline |
| (Control | 10 ml/kg/day | IP | A | 0.52% ethanol in wfi |
| group) | | | | |
| G2 | 0.90 mg/kg/day | IV | A | PM00104 |
| G3 | 16 mg/kg/day | IP | A | Docetaxel |
| G4 | 8 mg/kg/day | IP | A | Docetaxel |
| G5 | 8 mg/kg/day | IP | A | Oxaliplatin |
| G6 | 4 mg/kg/day | IP | A | Oxaliplatin |
| G7 | 0.90 mg/kg/day | IV | A | PM00104 |
| | 16 mg/kg/day | IP | A | Docetaxel |
| G8 | 0.90 mg/kg/day | IV | A | PM00104 |
| | 8 mg/kg/day | IP | A | Docetaxel |
| G9 | 0.90 mg/kg/day | IV | A | PM00104 |
| | 8 mg/kg/day | IP | A | Oxaliplatin |
| G10 | 0.90 mg/kg/day | IV | A | PM00104 |
| | 4 mg/kg/day | IP | A | Oxaliplatin |

A: DPI 20, 27, and 34;
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and docetaxel or PM00104 and oxaliplatin) against docetaxel or oxaliplatin mean tumor weight, respectively, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

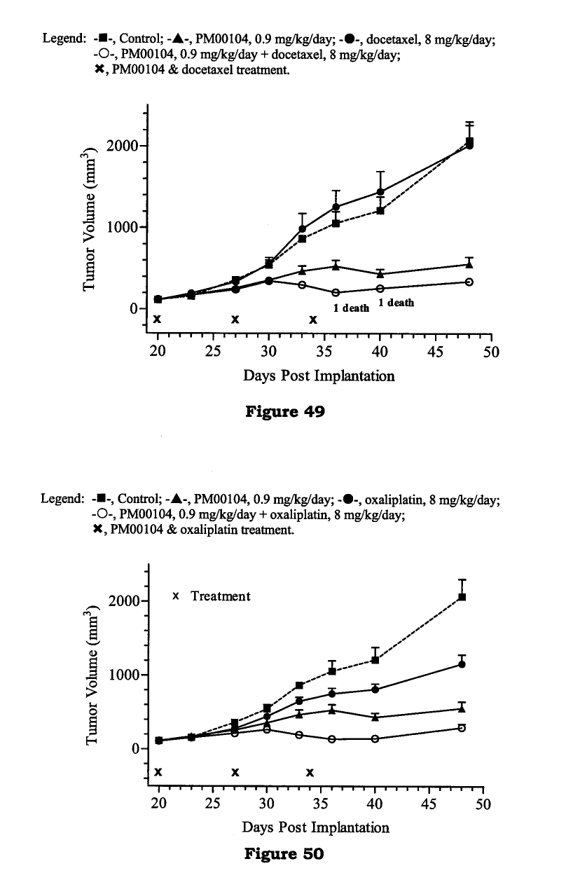

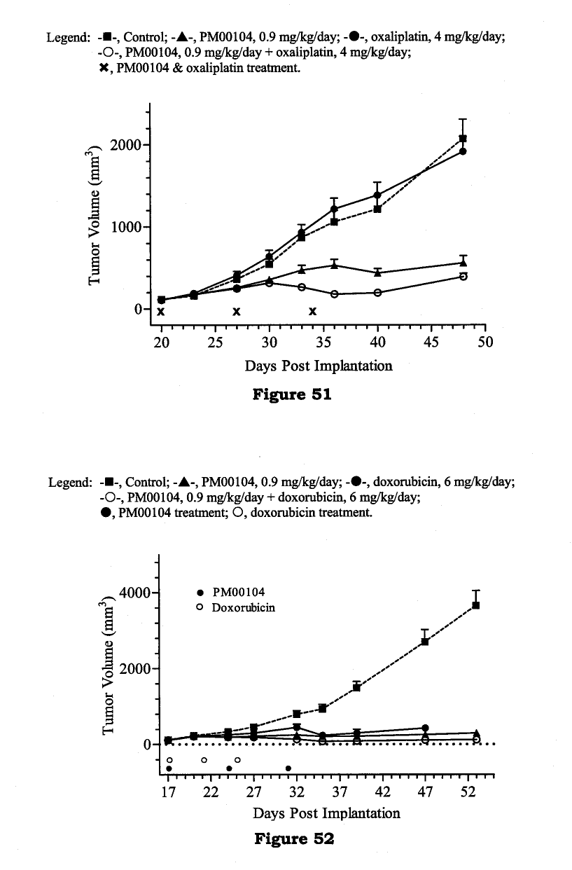

Table 42 reports the % T/C values obtained with each of the treatments and FIG. 48-51 show the tumor volume evaluation (mean±SEM) of MRI-H-254 tumors in mice treated with control (vehicle), PM00104, docetaxel, oxaliplatin, and the corresponding combinations.

TABLE 42

| | % T/C on day | | | | | | | |
|---|---|---|---|---|---|---|---|---|
| Group | 20 | 23 | 27 | 30 | 33 | 36 | 40 | 48 |
| G1 (Control | — | — | — | — | — | — | — | — |
| group) | | | | | | | | |
| G2 | 100.2 | 109.3 | 71.6 | 65.4 | 54.2 | 50.0 | 36.1 | 26.9 |
| G3 | 101.4 | 115.4 | 90.9 | 117.1 | 115.8 | 108.4 | 100.4 | 76.7 |
| G4 | 97.7 | 122.8 | 93.1 | 102.6 | 114.1 | 119.2 | 120.1 | 97.3 |
| G5 | 95.1 | 100.9 | 77.2 | 81.7 | 75.0 | 71.1 | 67.5 | 55.9 |
| G6 | 94.5 | 118.5 | 113.6 | 117.1 | 107.6 | 115.1 | 115.0 | 92.4 |
| G7 | 95.7 | 96.7 | 72.9 | 56.3 | 25.4 | 15.4 | 10.9 | 8.9 |
| G8 | 101.6 | 109.4 | 67.0 | 63.8 | 34.5 | 19.5 | 21.3 | 16.5 |
| G9 | 98.3 | 99.0 | 59.7 | 49.0 | 22.4 | 13.2 | 12.2 | 14.3 |
| G10 | 95.3 | 111.9 | 68.4 | 57.9 | 30.3 | 16.7 | 15.9 | 18.7 |

Table 43 shows the % of tumor growth inhibition of PM00104 and docetaxel administered as single agents and in combination at a dose of 0.9 mg/kg/day of PM00104 and 16 mg/kg/day of docetaxel. Additionally, the potentiation and the degree of additivity of the combination of PM00104 with docetaxel at said doses are provided.

TABLE 43

| | % Inhibition | | | Expected | Actual | | Degree of |
|---|---|---|---|---|---|---|---|
| Day | G2 | G3 | G7 | Response | Response | Potentiation | Response |
| 20 | −0.2 | −1.4 | 4.3 | −1.6 | 5.9 | yes | Additive |
| 23 | −9.3 | −15.4 | 3.3 | −24.7 | 28.0 | yes | Greater than additive |
| 27 | 28.4 | 9.1 | 27.1 | 37.5 | −10.4 | no | — |
| 30 | 34.6 | −17.1 | 43.7 | 17.5 | 26.2 | yes | Greater than additive |
| 33 | 45.8 | −15.8 | 74.6 | 30.0 | 44.6 | yes | Greater than additive |
| 36 | 50.0 | −8.4 | 84.6 | 41.6 | 43.0 | yes | Greater than additive |
| 40 | 63.9 | −0.4 | 89.1 | 63.5 | 25.6 | yes | Greater than additive |
| 48 | 73.1 | 23.3 | 91.1 | 96.4 | −5.3 | yes | Additive |

Table 44 shows the % of tumor growth inhibition of PM00104 and docetaxel administered as single agents and in combination at a dose of 0.9 mg/kg/day of PM00104 and 8 mg/kg/day of docetaxel. Additionally, the potentiation and the degree of additivity of the combination of PM00104 with docetaxel at said doses are provided.

TABLE 44

| | % Inhibition | | | Expected | Actual | | Degree of |
|---|---|---|---|---|---|---|---|
| Day | G2 | G4 | G8 | Response | Response | Potentiation | Response |
| 20 | −0.2 | 2.3 | −1.6 | 2.1 | −3.7 | no | — |
| 23 | −9.3 | −22.8 | −9.4 | −32.1 | 22.7 | no | — |
| 27 | 28.4 | 6.9 | 33.0 | 35.3 | −2.3 | yes | Additive |
| 30 | 34.6 | −2.6 | 36.2 | 32.0 | 4.2 | yes | Additive |
| 33 | 45.8 | −14.1 | 65.5 | 31.7 | 33.8 | yes | Greater than additive |
| 36 | 50.0 | −19.2 | 80.5 | 30.8 | 49.7 | yes | Greater than additive |
| 40 | 63.9 | −20.1 | 78.7 | 43.8 | 34.9 | yes | Greater than additive |
| 48 | 73.1 | 2.7 | 83.5 | 75.8 | 7.7 | yes | Greater than additive |

Table 45 shows the % of tumor growth inhibition of PM00104 and oxaliplatin administered as single agents and in combination at a dose of 0.9 mg/kg/day of PM00104 and 8 mg/kg/day of oxaliplatin. Additionally, the potentiation and the degree of additivity of the combination of PM00104 with oxaliplatin at said doses are provided.

TABLE 45

| | % Inhibition | | | Expected | Actual | | Degree of |
|---|---|---|---|---|---|---|---|
| Day | G2 | G5 | G9 | Response | Response | Potentiation | Response |
| 20 | −0.2 | 4.9 | 1.7 | 4.7 | −3.0 | no | — |
| 23 | −9.3 | −0.9 | 1.0 | −10.2 | 11.2 | no | — |
| 27 | 28.4 | 22.8 | 40.3 | 51.2 | −10.9 | yes | Less than additive |
| 30 | 34.6 | 18.3 | 51.0 | 52.9 | −1.9 | yes | Additive |
| 33 | 45.8 | 25.0 | 77.6 | 70.8 | 6.8 | yes | Greater than additive |
| 36 | 50.0 | 28.9 | 86.8 | 78.9 | 7.9 | yes | Greater than additive |
| 40 | 63.9 | 32.5 | 87.8 | 96.4 | −8.6 | yes | Less than additive |
| 48 | 73.1 | 44.1 | 85.7 | 117.2 | −31.5 | yes | Less than additive |

Table 46 shows the % of tumor growth inhibition of PM00104 and oxaliplatin administered as single agents and in combination at a dose of 0.9 mg/kg/day of PM00104 and 4 mg/kg/day of oxaliplatin. Additionally, the potentiation and the degree of additivity of the combination of PM00104 with oxaliplatin at said doses are provided.

TABLE 46

| | % Inhibition | | | Expected | Actual | | Degree of |
|---|---|---|---|---|---|---|---|
| Day | G2 | G6 | G10 | Response | Response | Potentiation | Response |
| 20 | −0.2 | 5.5 | 4.7 | 5.3 | −0.6 | no | — |
| 23 | −9.3 | −18.5 | −11.9 | −27.8 | 15.9 | no | — |
| 27 | 28.4 | −13.6 | 31.6 | 14.8 | 16.8 | yes | Greater than additive |
| 30 | 34.6 | −17.1 | 42.1 | 17.5 | 24.6 | yes | Greater than additive |
| 33 | 45.8 | −7.6 | 69.7 | 38.2 | 31.5 | yes | Greater than additive |
| 36 | 50.0 | −15.1 | 83.3 | 34.9 | 48.4 | yes | Greater than additive |
| 40 | 63.9 | −15.0 | 84.1 | 48.9 | 35.2 | yes | Greater than additive |
| 48 | 73.1 | 7.6 | 81.3 | 80.7 | 0.6 | yes | Additive |

According to this assay it was found that:

a. The combination of PM00104 and docetaxel resulted in a statistically significant (p<0.001) potentiation of antitumor activity. This potentiation was found to be greater than additive.
b. The combination of PM00104 and oxaliplatin resulted in a statistically significant (p<0.001) potentiation of antitumor activity. This potentiation was graded as greater than additive.

### Example 13

In vivo studies to determine the effect of PM00104 in combination with doxorubicin and paclitaxel in human gastric tumor xenografts.

The aim of these studies was to evaluate the ability of PM00104 to potentiate the antitumor activity of doxorubicin and paclitaxel by using a xenograft model of human gastric carcinoma.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation with tumor fragments. The Vehicle Control group contained 11 mice, groups 2-6 contained 8 mice/group, and the rest of groups contained 9 mice/group.

The tumor model used in these studies was MRI-H-254 cell line, which was obtained from the DCT Tumor Bank. This cell line was grown and implanted to the animals as described in Example 11.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 17.

PM00104 was provided in the form of vials of lyophilized PM00104 powder which was reconstituted with water for injection. Doxorubicin was provided in the form of a lyophilized powder, which was reconstituted in 0.9% saline. Paclitaxel was provided as solution which was further diluted with 0.9% saline.

Study groups and treatment regimens are listed in table 47.

TABLE 47

| Group | Dose | Route | Schedule | Test material |
|---|---|---|---|---|
| G1 | 10 ml/kg/day | IV | A | 0.18% Placebo in Saline |
| (Control | 10 ml/kg/day | IP | B | 0.9% Saline |
| group) | | | | |
| G2 | 0.90 mg/kg/day | IV | A | PM00104 |
| G3 | 0.45 mg/kg/day | IV | A | PM00104 |
| G4 | 0.23 mg/kg/day | IV | A | PM00104 |
| G5 | 6 mg/kg/day | IP | B | Doxorubicin |
| G6 | 12.5 mg/kg/day | IP | B | Paclitaxel |
| G7 | 0.90 mg/kg/day | IV | A | PM00104 |
| | 6 mg/kg/day | IP | B | Doxorubicin |
| G8 | 0.90 mg/kg/day | IV | A | PM00104 |
| | 12.5 mg/kg/day | IP | B | Paclitaxel |
| G9 | 0.45 mg/kg/day | IV | A | PM00104 |
| | 6 mg/kg/day | IP | B | Doxorubicin |
| G10 | 0.45 mg/kg/day | IV | A | PM00104 |
| | 12.5 mg/kg/day | IP | B | Paclitaxel |
| G11 | 0.23 mg/kg/day | IV | A | PM00104 |
| | 6 mg/kg/day | IP | B | Doxorubicin |
| G12 | 0.23 mg/kg/day | IV | A | PM00104 |
| | 12.5 mg/kg/day | IP | B | Paclitaxel |

A: DPI 17, 24, and 31;
B: DPI 17, 21, 25
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and doxorubicin or PM00104 and paclitaxel) against doxorubicin or paclitaxel mean tumor weight, respectively, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

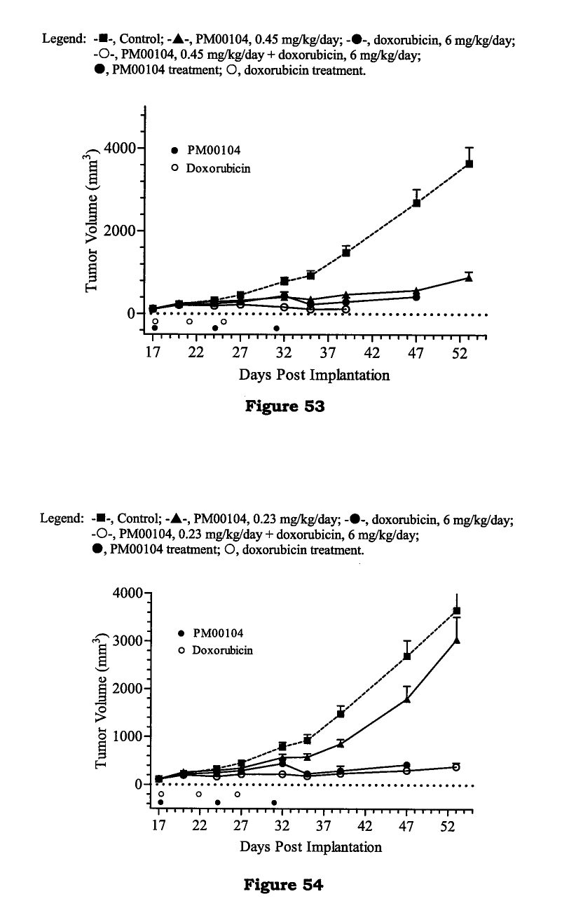

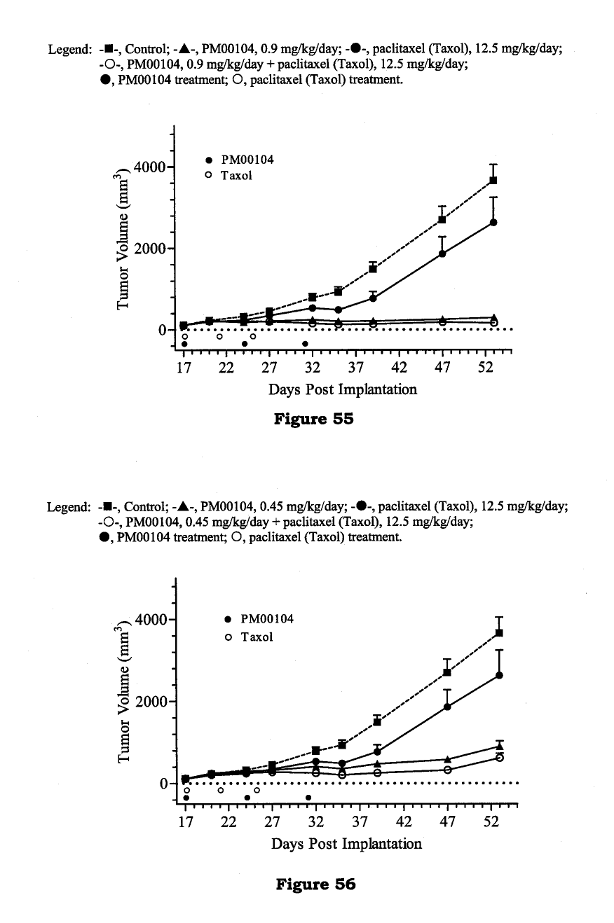

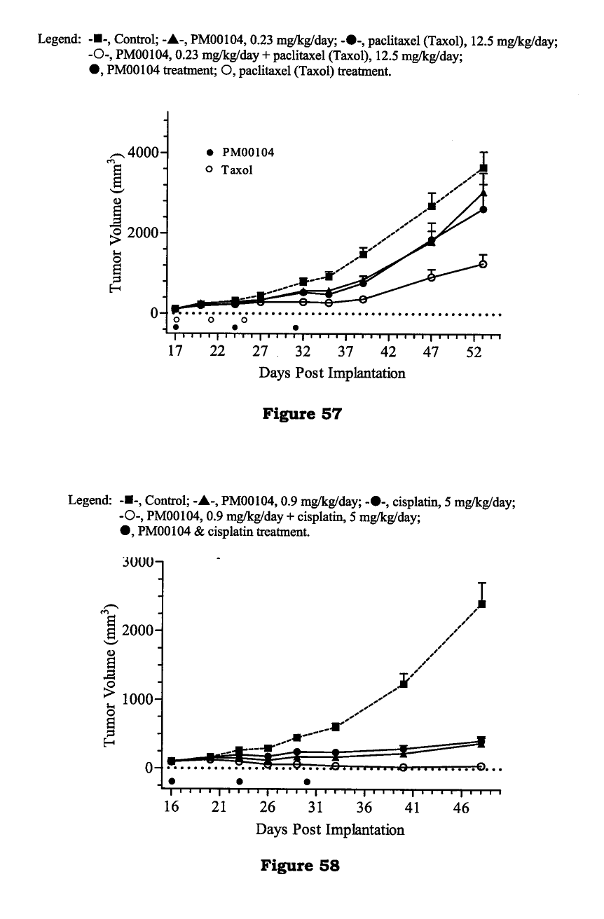

Table 48 reports the % T/C values obtained with each of the treatments and FIG. 52-57 show the tumor volume evaluation (mean±SEM) of MRI-H-254 tumors in mice treated with control (vehicle), PM00104, doxorubicin, paclitaxel, and the corresponding combinations.

TABLE 48

| | % T/C on day | | | | | | | |
|---|---|---|---|---|---|---|---|---|
| Group | 17 | 20 | 24 | 27 | 32 | 35 | 39 | 47 |
| G1 (Control | — | — | — | — | — | — | — | — |
| group) | | | | | | | | |
| G2 | 98.5 | 95.6 | 56.9 | 46.4 | 31.2 | 22.6 | 14.5 | 9.5 |
| G3 | 100.0 | 109.2 | 91.3 | 71.0 | 51.8 | 38.4 | 31.8 | 21.5 |
| G4 | 97.5 | 113.0 | 89.9 | 75.9 | 72.7 | 62.4 | 57.6 | 66.8 |
| G5 | 98.2 | 93.5 | 76.2 | 64.4 | 56.3 | 24.8 | 19.7 | 15.7 |
| G6 | 99.5 | 86.2 | 71.9 | 77.2 | 67.9 | 52.3 | 51.7 | 69.0 |
| G7 | 96.6 | 90.4 | 57.4 | 40.3 | 16.4 | 8.5 | 5.7 | 4.1 |
| G8 | 94.1 | 96.4 | 64.4 | 44.8 | 20.2 | 13.8 | 9.2 | 6.9 |
| G9 | 100.0 | 92.1 | 60.0 | 50.2 | 21.3 | 12.4 | 8.0 | — |
| G10 | 99.9 | 102.2 | 76.4 | 61.2 | 32.5 | 22.1 | 17.2 | 12.0 |
| G11 | 98.0 | 86.6 | 51.3 | 48.1 | 28.4 | 19.7 | 16.1 | 11.3 |
| G12 | 97.2 | 94.1 | 72.1 | 63.6 | 37.3 | 29.6 | 24.7 | 34.2 |

Table 49 shows the % of tumor growth inhibition of PM00104 and doxorubicin administered as single agents and in combination at a dose of 0.9 mg/kg/day of PM00104 and 6 mg/kg/day of doxorubicin. Additionally, the potentiation and the degree of additivity of the combination of PM00104 with doxorubicin at said doses are provided.

TABLE 49

| | % Inhibition | | | Expected | Actual | Potenti- | Degree of |
|---|---|---|---|---|---|---|---|
| Day | G2 | G5 | G7 | Response | Response | ation | Response |
| 14 | 1.5 | 1.8 | 3.4 | 3.3 | 0.1 | yes | Additive |
| 20 | 4.4 | 6.5 | 9.6 | 10.9 | −1.3 | yes | Additive |
| 24 | 43.1 | 23.8 | 42.6 | 66.9 | −24.3 | no | — |
| 27 | 53.6 | 35.6 | 59.7 | 89.2 | −29.5 | yes | Less than |
| | | | | | | | additive |
| 32 | 68.8 | 43.7 | 83.6 | 112.5 | −28.9 | yes | Less than |
| | | | | | | | additive |
| 35 | 77.4 | 75.2 | 91.5 | 152.6 | −61.1 | yes | Less than |
| | | | | | | | additive |
| 39 | 85.5 | 80.3 | 94.3 | 165.8 | −71.5 | yes | Less than |
| | | | | | | | additive |
| 47 | 90.5 | 84.3 | 95.9 | 174.8 | −78.9 | yes | Less than |
| | | | | | | | additive |

Table 50 shows the % of tumor growth inhibition of PM00104 and doxorubicin administered as single agents and in combination at a dose of 0.45 mg/kg/day of PM00104 and 6 mg/kg/day of doxorubicin. Additionally, the potentiation and the degree of additivity of the combination of PM00104 with doxorubicin at said doses are provided.

TABLE 50

| | % Inhibition | | | Expected | Actual | Potenti- | Degree of |
|---|---|---|---|---|---|---|---|
| Day | G3 | G5 | G9 | Response | Response | ation | Response |
| 14 | 0.0 | 1.8 | 0.0 | 1.8 | −1.8 | no | — |
| 20 | −9.2 | 6.5 | 7.9 | −2.7 | 10.6 | yes | Additive |
| 24 | 8.7 | 23.8 | 40.0 | 32.5 | 7.5 | yes | Additive |
| 27 | 29.0 | 35.6 | 49.8 | 64.6 | −14.8 | yes | Less than |
| | | | | | | | additive |
| 32 | 48.2 | 43.7 | 78.7 | 91.9 | −13.2 | yes | Less than |
| | | | | | | | additive |
| 35 | 61.6 | 75.2 | 87.6 | 136.8 | −49.2 | yes | Less than |
| | | | | | | | additive |
| 39 | 68.2 | 80.3 | 92.0 | 148.5 | −56.5 | yes | Less than |
| | | | | | | | additive |

Table 51 shows the % of tumor growth inhibition of PM00104 and doxorubicin administered as single agents and in combination at a dose of 0.23 mg/kg/day of PM00104 and 6 mg/kg/day of doxorubicin. Additionally, the potentiation and the degree of additivity of the combination of PM00104 with doxorubicin at said doses are provided.

TABLE 51

| | % Inhibition | | | Expected | Actual | | Degree of |
|---|---|---|---|---|---|---|---|
| Day | G4 | G5 | G11 | Response | Response | Potentiation | Response |
| 14 | 2.5 | 1.8 | 2.0 | 4.3 | −2.3 | no | Additive |
| 20 | −13.0 | 6.5 | 13.4 | −6.5 | 19.9 | yes | Greater than additive |
| 24 | 10.1 | 23.8 | 48.7 | 33.9 | 14.8 | yes | Greater than additive |
| 27 | 24.1 | 35.6 | 51.9 | 59.7 | −7.8 | yes | Additive |
| 32 | 27.3 | 43.7 | 71.6 | 71.0 | 0.6 | yes | Additive |
| 35 | 37.6 | 75.2 | 80.3 | 112.8 | −32.5 | yes | Less than additive |
| 39 | 42.4 | 80.3 | 83.9 | 122.7 | −38.8 | yes | Less than additive |
| 47 | 33.2 | 84.3 | 88.7 | 117.5 | −28.8 | yes | Less than additive |

Table 52 shows the % of tumor growth inhibition of PM00104 and paclitaxel administered as single agents and in combination at a dose of 0.90 mg/kg/day of PM00104 and 12.5 mg/kg/day of paclitaxel. Additionally, the potentiation and the degree of additivity of the combination of PM00104 with paclitaxel at said doses are provided.

TABLE 52

| | % Inhibition | | | Expected | Actual | | Degree of |
|---|---|---|---|---|---|---|---|
| Day | G2 | G6 | G8 | Response | Response | Potentiation | Response |
| 14 | 1.5 | 0.5 | 5.9 | 2.0 | 3.9 | no | — |
| 20 | 4.4 | 13.8 | 3.6 | 18.2 | −14.6 | no | — |
| 24 | 43.1 | 28.1 | 35.6 | 71.2 | −35.6 | no | — |
| 27 | 53.6 | 22.8 | 55.2 | 76.4 | −21.2 | yes | Less than additive |
| 32 | 68.8 | 32.1 | 79.8 | 100.9 | −21.1 | yes | Less than additive |
| 35 | 77.4 | 47.7 | 86.2 | 125.1 | −38.9 | yes | Less than additive |
| 39 | 85.5 | 48.3 | 90.8 | 133.8 | −43.0 | yes | Less than additive |
| 47 | 90.5 | 31.0 | 93.1 | 121.5 | −28.4 | yes | Less than additive |

Table 53 shows the % of tumor growth inhibition of PM00104 and paclitaxel administered as single agents and in combination at a dose of 0.45 mg/kg/day of PM00104 and 12.5 mg/kg/day of paclitaxel. Additionally, the potentiation and the degree of additivity of the combination of PM00104 with paclitaxel at said doses are provided.

TABLE 53

| | % Inhibition | | | Expected | Actual | | Degree of |
|---|---|---|---|---|---|---|---|
| Day | G3 | G6 | G10 | Response | Response | Potentiation | Response |
| 14 | 0.0 | 0.5 | 0.1 | 0.5 | −0.4 | no | — |
| 20 | −9.2 | 13.8 | −2.2 | 4.6 | −6.8 | no | — |
| 24 | 8.7 | 28.1 | 23.6 | 36.8 | −13.2 | no | — |
| 27 | 29.0 | 22.8 | 38.8 | 51.8 | −13.0 | yes | Less than additive |
| 32 | 48.2 | 32.1 | 67.5 | 80.3 | −12.8 | yes | Less than additive |
| 35 | 61.6 | 47.7 | 77.9 | 109.3 | −31.4 | yes | Less than additive |
| 39 | 68.2 | 48.3 | 82.8 | 116.5 | −33.7 | yes | Less than additive |
| 47 | 78.5 | 31.0 | 88.0 | 109.5 | −21.5 | yes | Less than additive |

Table 54 shows the % of tumor growth inhibition of PM00104 and paclitaxel administered as single agents and in combination at a dose of 0.23 mg/kg/day of PM00104 and 12.5 mg/kg/day of paclitaxel. Additionally, the potentiation and the degree of additivity of the combination of PM00104 with paclitaxel at said doses are provided.

TABLE 54

| | % Inhibition | | | Expected | Actual | | Degree of |
|---|---|---|---|---|---|---|---|
| Day | G4 | G6 | G12 | Response | Response | Potentiation | Response |
| 14 | 2.5 | 0.5 | 2.8 | 3.0 | −0.2 | no | — |
| 20 | −13.0 | 13.8 | 5.9 | 0.8 | 5.1 | no | — |
| 24 | 10.1 | 28.1 | 27.9 | 38.2 | −10.3 | no | — |
| 27 | 24.1 | 22.8 | 36.4 | 46.9 | −10.5 | yes | Less than additive |
| 32 | 27.3 | 32.1 | 62.7 | 59.4 | 3.3 | yes | Additive |
| 35 | 37.6 | 47.7 | 70.4 | 85.3 | −14.9 | yes | Less than additive |
| 39 | 42.4 | 48.3 | 75.3 | 90.7 | −15.4 | yes | Less than additive |
| 47 | 33.2 | 31.0 | 65.8 | 64.2 | 1.6 | yes | Additive |

According to this assay it was found that:

a. The combination of PM00104 and doxorubicin resulted in a potentiation of antitumor activity. This potentiation was found to be less than additive. In addition, in the doses and schedules evaluated the combination resulted in some toxicity signs with a 50% of mortality among the animals.
b. The combination of PM00104 and paclitaxel resulted in a statistically significant (p<0.001) potentiation of antitumor activity. This potentiation was graded as less than additive, being the best results observed at the lower dose of PM00104.

### Example 14

In vivo studies to determine the effect of PM00104 in combination with cisplatin, paclitaxel, and irinotecan in human gastric tumor xenografts.

The aim of these studies was to evaluate the ability of PM00104 to potentiate the antitumor activity of cisplatin, paclitaxel, and irinotecan by using a xenograft model of human gastric carcinoma.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation with tumor fragments. The Vehicle Control group contained 15 mice and the treated groups had each 9 mice/group.

The tumor model used in these studies was MRI-H-254 cell line, which was obtained from the DCT Tumor Bank. This cell line was grown and implanted to the animals as described in Example 11.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 16.

PM00104 was provided in the form of vials of lyophilized PM00104 powder which was reconstituted with water for injection. Cisplatin and paclitaxel were provided as solutions which were further diluted with 0.9% saline. Irinotecan was provided in the form a solution containing Irinotecan HCl trihydrate, which was diluted in 0.9% sterile saline.

Study groups and treatment regimens are listed in table 55.

TABLE 55

| Group | Dose | Route | Schedule | Test material |
|---|---|---|---|---|
| G1 | 10 ml/kg/day | IV | A | 0.18% Placebo in Saline |
| (Control | 10 ml/kg/day | IP | B | 0.9% Saline |
| group) | | | | |
| G2 | 0.90 mg/kg/day | IV | A | PM00104 |
| G3 | 5 mg/kg/day | IV | A | Cisplatin |
| G4 | 3 mg/kg/day | IV | A | Cisplatin |
| G5 | 18 mg/kg/day | IP | A | Irinotecan |
| G6 | 10 mg/kg/day | IP | A | Irinotecan |
| G7 | 25 mg/kg/day | IP | B | Paclitaxel |
| G8 | 12.5 mg/kg/day | IP | B | Paclitaxel |
| G9 | 0.90 mg/kg/day | IV | A | PM00104 |
| | 5 mg/kg/day | IP | A | Cisplatin |
| G10 | 0.90 mg/kg/day | IV | A | PM00104 |
| | 3 mg/kg/day | IP | A | Cisplatin |
| G11 | 0.90 mg/kg/day | IV | A | PM00104 |
| | 18 mg/kg/day | IP | A | Irinotecan |
| G12 | 0.90 mg/kg/day | IV | A | PM00104 |
| | 10 mg/kg/day | IP | A | Irinotecan |
| G13 | 0.90 mg/kg/day | IV | A | PM00104 |
| | 25 mg/kg/day | IP | B | Paclitaxel |
| G14 | 0.90 mg/kg/day | IV | A | PM00104 |
| | 12.5 mg/kg/day | IP | B | Paclitaxel |

A: DPI 16, 23, and 30;
B: DPI 16, 20, 24
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and cisplatin, PM00104 and irinotecan or PM00104 and paclitaxel) against cisplatin, irinotecan or paclitaxel mean tumor weight, respectively, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

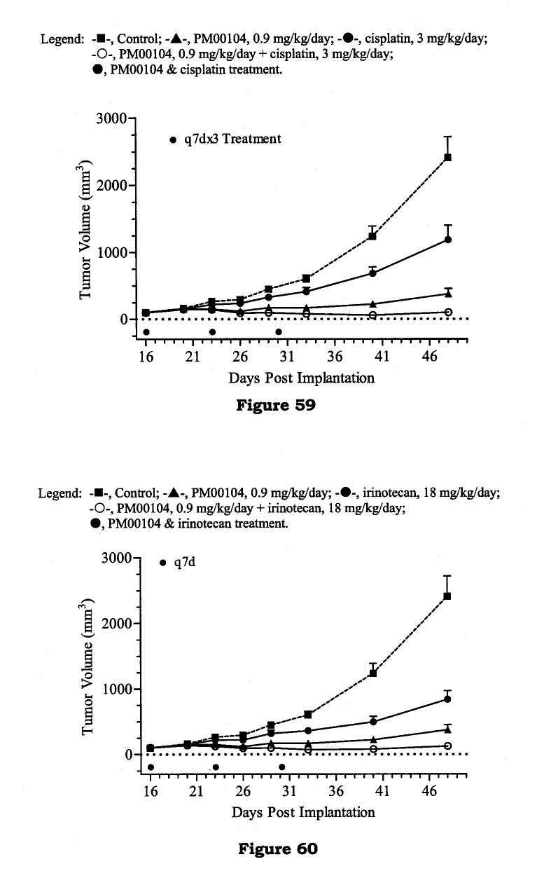

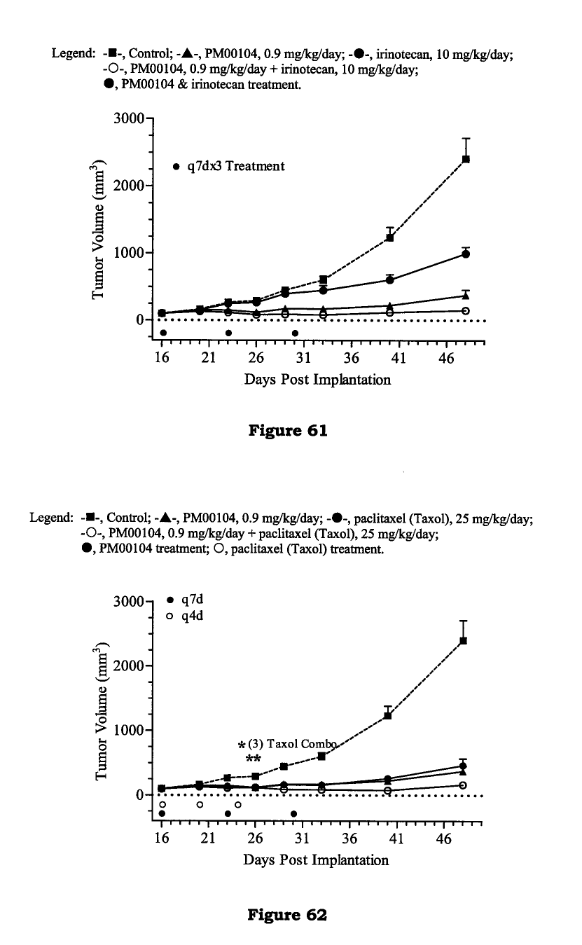

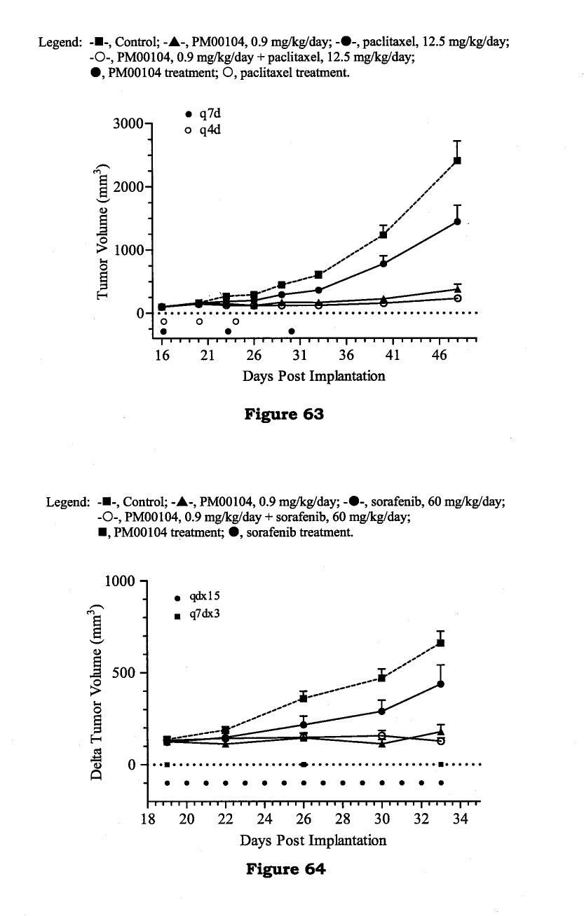

Table 56 reports the % T/C values obtained with each of the treatments and FIG. 58-63 show the tumor volume evaluation (mean±SEM) of MRI-H-254 tumors in mice treated with control (vehicle), PM00104, ciaplatin, irinotecan, paclitaxel, and the corresponding combinations.

TABLE 56

| | % T/C on day | | | | | | | |
|---|---|---|---|---|---|---|---|---|
| Group | 16 | 20 | 23 | 26 | 29 | 33 | 40 | 48 |
| G1 (Control | — | — | — | — | — | — | — | — |
| group) | | | | | | | | |
| G2 | 98.0 | 93.9 | 56.1 | 40.6 | 38.3 | 28.0 | 18.2 | 15.6 |
| G3 | 100.5 | 97.8 | 75.3 | 59.9 | 54.3 | 39.5 | 23.8 | 17.1 |
| G4 | 98.4 | 93.4 | 83.0 | 81.5 | 73.0 | 68.6 | 55.3 | 49.2 |
| G5 | 98.1 | 92.1 | 84.0 | 75.0 | 71.0 | 60.2 | 40.1 | 34.9 |
| G6 | 98.4 | 92.5 | 93.0 | 90.2 | 88.6 | 74.3 | 49.2 | 41.4 |
| G7 | 96.1 | 78.9 | 51.6 | 40.5 | 36.4 | 26.1 | 21.2 | 19.3 |
| G8 | 99.9 | 98.6 | 69.6 | 68.5 | 65.3 | 60.7 | 63.1 | 59.9 |
| G9 | 96.8 | 77.6 | 37.9 | 19.7 | 13.8 | 6.4 | 2.0 | 1.9 |
| G10 | 96.7 | 89.3 | 54.6 | 31.7 | 21.7 | 12.8 | 4.7 | 4.2 |
| G11 | 101.3 | 83.8 | 47.1 | 31.8 | 22.4 | 12.1 | 6.6 | 5.3 |
| G12 | 101.9 | 79.5 | 44.2 | 28.3 | 19.9 | 13.5 | 9.7 | 6.2 |
| G13 | 95.4 | 80.0 | 42.3 | 41.1 | 19.6 | 14.2 | 6.3 | 6.8 |
| G14 | 99.7 | 87.6 | 47.7 | 41.0 | 27.4 | 20.4 | 12.6 | 9.5 |

Table 57 shows the % of tumor growth inhibition of PM00104 and cisplatin administered as single agents and in combination at a dose of 0.9 mg/kg/day of PM00104 and 5 mg/kg/day of cisplatin. Additionally, the potentiation and the degree of additivity of the combination of PM00104 with cisplatin at said doses are provided.

TABLE 57

| | % Inhibition | | | Expected | Actual | | Degree of |
|---|---|---|---|---|---|---|---|
| Day | G2 | G3 | G9 | Response | Response | Potentiation | Response |
| 16 | 2.0 | −0.5 | 3.2 | 1.5 | 1.7 | no | — |
| 20 | 6.1 | 2.2 | 22.4 | 8.3 | 14.1 | yes | Greater than additive |
| 23 | 43.9 | 24.7 | 62.1 | 68.6 | −6.5 | yes | Additive |
| 26 | 59.4 | 40.1 | 80.3 | 99.5 | −19.2 | yes | Less than additive |
| 29 | 61.7 | 45.7 | 86.2 | 107.4 | −21.2 | yes | Less than additive |
| 33 | 72.0 | 60.5 | 93.6 | 132.5 | −38.9 | yes | Less than additive |
| 40 | 81.8 | 76.2 | 98.0 | 158.0 | −60.0 | yes | Less than additive |
| 48 | 84.4 | 82.9 | 98.1 | 167.3 | −69.2 | yes | Less than additive |

Table 58 shows the % of tumor growth inhibition of PM00104 and cisplatin administered as single agents and in combination at a dose of 0.9 mg/kg/day of PM00104 and 3 mg/kg/day of cisplatin. Additionally, the potentiation and the degree of additivity of the combination of PM00104 with cisplatin at said doses are provided.

TABLE 58

| | % Inhibition | | | Expected | Actual | | Degree of |
|---|---|---|---|---|---|---|---|
| Day | G2 | G4 | G10 | Response | Response | Potentiation | Response |
| 16 | 2.0 | 1.6 | 3.3 | 3.6 | −0.3 | no | — |
| 20 | 6.1 | 6.6 | 10.7 | 12.7 | −2.0 | yes | Additive |
| 23 | 43.9 | 17.0 | 45.4 | 60.9 | −15.5 | yes | Less than additive |
| 26 | 59.4 | 18.5 | 68.3 | 77.9 | −9.6 | yes | Additive |
| 29 | 61.7 | 27.0 | 78.3 | 88.7 | −10.4 | yes | Less than additive |
| 33 | 72.0 | 31.4 | 87.2 | 103.4 | −16.2 | yes | Less than additive |
| 40 | 81.8 | 44.7 | 95.3 | 126.5 | −31.2 | yes | Less than additive |
| 48 | 84.4 | 50.8 | 95.8 | 135.2 | −39.4 | yes | Less than additive |

Table 59 shows the % of tumor growth inhibition of PM00104 and irinotecan administered as single agents and in combination at a dose of 0.9 mg/kg/day of PM00104 and 18 mg/kg/day of irinotecan. Additionally, the potentiation and the degree of additivity of the combination of PM00104 with irinotecan at said doses are provided.

TABLE 59

| | % Inhibition | | | Expected | Actual | | Degree of |
|---|---|---|---|---|---|---|---|
| Day | G2 | G5 | G11 | Response | Response | Potentiation | Response |
| 16 | 2.0 | 1.9 | −1.3 | 3.9 | −5.2 | no | — |
| 20 | 6.1 | 7.9 | 16.2 | 14.0 | 2.2 | yes | Additive |
| 23 | 43.9 | 16.0 | 52.9 | 59.9 | −7.0 | yes | Additive |
| 26 | 59.4 | 25.0 | 68.2 | 84.4 | −16.2 | yes | Less than additive |
| 29 | 61.7 | 29.0 | 77.6 | 90.7 | −13.1 | yes | Less than additive |
| 33 | 72.0 | 39.8 | 87.9 | 111.8 | −23.9 | yes | Less than additive |
| 40 | 81.8 | 59.9 | 93.4 | 141.7 | −48.3 | yes | Less than additive |
| 48 | 84.4 | 65.1 | 94.7 | 149.5 | −54.8 | yes | Less than additive |

Table 60 shows the % of tumor growth inhibition of PM00104 and irinotecan administered as single agents and in combination at a dose of 0.9 mg/kg/day of PM00104 and 10 mg/kg/day of irinotecan. Additionally, the potentiation and the degree of additivity of the combination of PM00104 with irinotecan at said doses are provided.

TABLE 60

| | % Inhibition | | | Expected | Actual | | Degree of |
|---|---|---|---|---|---|---|---|
| Day | G2 | G6 | G12 | Response | Response | Potentiation | Response |
| 16 | 2.0 | 1.6 | −1.9 | 3.6 | −5.5 | no | — |
| 20 | 6.1 | 7.5 | 20.5 | 13.6 | 6.9 | yes | Additive |
| 23 | 43.9 | 7.0 | 55.8 | 50.9 | 4.9 | yes | Additive |
| 26 | 59.4 | 9.8 | 71.7 | 69.2 | 2.5 | yes | Additive |
| 29 | 61.7 | 11.4 | 80.1 | 73.1 | 7.0 | yes | Additive |
| 33 | 72.0 | 25.7 | 86.5 | 97.7 | −11.2 | yes | Less than additive |
| 40 | 81.8 | 50.8 | 90.3 | 132.6 | −42.3 | yes | Less than additive |
| 48 | 84.4 | 58.6 | 93.8 | 143.0 | −49.2 | yes | Less than additive |

Table 61 shows the % of tumor growth inhibition of PM00104 and paclitaxel administered as single agents and in combination at a dose of 0.9 mg/kg/day of PM00104 and 25 mg/kg/day of paclitaxel. Additionally, the potentiation and the degree of additivity of the combination of PM00104 with paclitaxel at said doses are provided.

TABLE 61

| | % Inhibition | | | Expected | Actual | | Degree of |
|---|---|---|---|---|---|---|---|
| Day | G2 | G7 | G13 | Response | Response | Potentiation | Response |
| 16 | 2.0 | 3.9 | 4.6 | 5.9 | −1.3 | no | — |
| 20 | 6.1 | 21.1 | 20 | 27.2 | −7.2 | no | — |
| 23 | 43.9 | 48.4 | 57.7 | 92.3 | −34.6 | yes | Less than additive |
| 26 | 59.4 | 59.5 | 58.9 | 118.9 | −60.0 | no | — |
| 29 | 61.7 | 63.6 | 80.4 | 125.3 | −44.9 | yes | Less than additive |
| 33 | 72.0 | 73.9 | 85.8 | 145.9 | −60.1 | yes | Less than additive |
| 40 | 81.8 | 78.8 | 93.7 | 160.6 | −66.9 | yes | Less than additive |
| 48 | 84.4 | 80.7 | 93.2 | 165.1 | −71.9 | yes | Less than additive |

Table 62 shows the % of tumor growth inhibition of PM00104 and paclitaxel administered as single agents and in combination at a dose of 0.9 mg/kg/day of PM00104 and 12.5 mg/kg/day of paclitaxel. Additionally, the potentiation and the degree of additivity of the combination of PM00104 with paclitaxel at said doses are provided.

TABLE 62

| | % Inhibition | | | Expected | Actual | | Degree of |
|---|---|---|---|---|---|---|---|
| Day | G2 | G8 | G14 | Response | Response | Potentiation | Response |
| 16 | 2.0 | 0.1 | 0.3 | 2.1 | −1.8 | no | — |
| 20 | 6.1 | 1.4 | 12.4 | 7.5 | 4.9 | yes | Additive |
| 23 | 43.9 | 30.4 | 52.3 | 74.3 | −22.0 | yes | Less than additive |
| 26 | 59.4 | 31.5 | 59.0 | 90.9 | −31.9 | no | — |
| 29 | 61.7 | 34.7 | 72.6 | 96.4 | −23.8 | yes | Less than additive |
| 33 | 72.0 | 39.3 | 79.6 | 111.3 | −31.7 | yes | Less than additive |
| 40 | 81.8 | 36.9 | 87.4 | 118.7 | −31.3 | yes | Less than additive |
| 48 | 84.4 | 40.1 | 90.5 | 124.5 | −34.0 | yes | Less than additive |

According to this assay it was found that:

a. The combination of PM00104 and cisplatin resulted in a statistically significant (p<0.001) potentiation of antitumor activity over results obtained with cisplatin as a single agent control group but not with PM00104 as a single agent control group, with the potentiation being graded as less than additive at the end of the experiment.
b. The combination of PM00104 and irinotecan resulted in a highly statistically significant (p<0.001) potentiation of antitumor activity over results obtained with irinotecan as a single agent control group but not with PM00104 as a single agent control group, with the potentiation being graded as less than additive at the end of the experiment.
c. The combination of PM00104 and paclitaxel resulted in a potentiation of antitumor activity, which was statistically significant (p<0.001) at a paclitaxel dose of 12.5 mg/kg/day. This potentiation was graded as less than additive.

### Example 15

In vivo studies to determine the effect of PM00104 in combination with Sorafenib in human hepatoma xenografts.

The aim of these studies was to evaluate the ability of PM00104 to potentiate the antitumor activity of sorafenib by using a xenograft model of human hepatoma.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation with a tumor cell suspension. The Vehicle Control group contained 15 mice and the treated groups had each 9 mice/group.

The tumor model used in these studies was HepG2 cell line, which was obtained from the ATCC (Manassas, Va.).

HepG2 cells were grown in MEM supplemented with 10% FBS, 1.5 g/L sodium bicarbonate, 0.1 mM non-essential amino acids, 1.0 mM sodium pyruvate, and 2 mM L-glutamine. Each animal was implanted SC on the right flank, using a 13G trochar, with 5×10⁶ HepG2 cells in a 0.2 mL suspension of 50% Matrigel and 50% serum free medium, without antibiotics. Bacterial cultures were performed on aliquots of the cells prepared for implantation. All cultures were negative for bacterial contamination at both 24 and 48 hours post-implant.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 19.

PM00104 was provided in the form of vials of lyophilized PM00104 powder which was reconstituted with water for injection. Sorafenib was provided in the form of a tablet which was dissolved in Cremophor EL/ethanol/water (CEW) (12.5, 12.5, 75) final proportion.

Study groups and treatment regimens are listed in table 63.

TABLE 63

| Group | Dose | Route | Schedule | Test material |
|---|---|---|---|---|
| G1 | 10 ml/kg/day | IV | A | 0.18% Placebo in Saline |
| (Control | 10 ml/kg/day | PO | B | 0.9% Saline |
| group) | | | | |
| G2 | 0.90 mg/kg/day | IV | A | PM00104 |
| G3 | 0.60 mg/kg/day | IV | A | PM00104 |
| G4 | 60 mg/kg/day | PO | B | Sorafenib |
| G5 | 30 mg/kg/day | PO | B | Sorafenib |
| G6 | 0.90 mg/kg/day | IV | A | PM00104 |
| | 60 mg/kg/day | PO | B | Sorafenib |
| G7 | 0.90 mg/kg/day | IV | A | PM00104 |
| | 30 mg/kg/day | PO | B | Sorafenib |
| G8 | 0.60 mg/kg/day | IV | A | PM00104 |
| | 60 mg/kg/day | PO | B | Sorafenib |
| G9 | 0.60 mg/kg/day | IV | A | PM00104 |
| | 30 mg/kg/day | PO | B | Sorafenib |

A: DPI 19, 26, and 33;
B: DPI 19-33
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and sorafenib) against sorafenib mean tumor weight, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

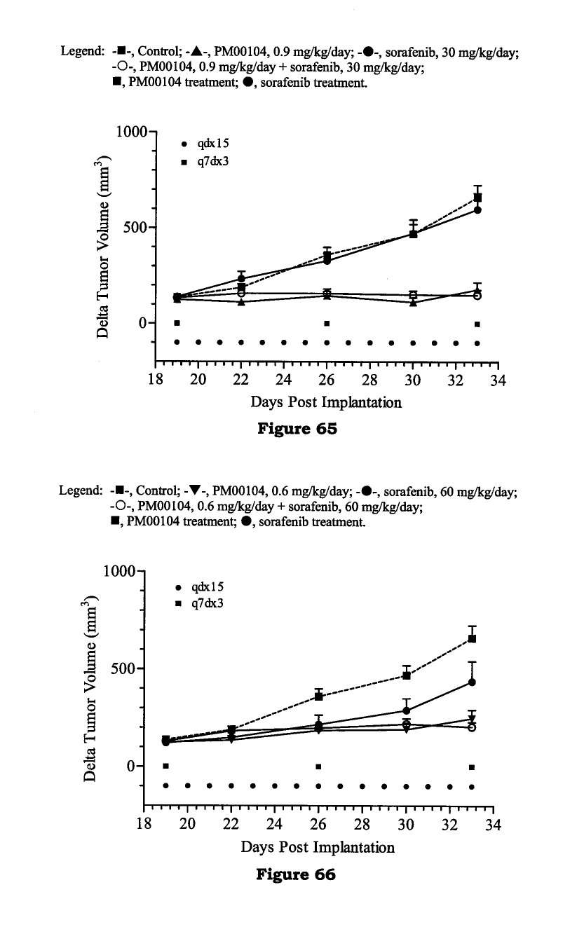

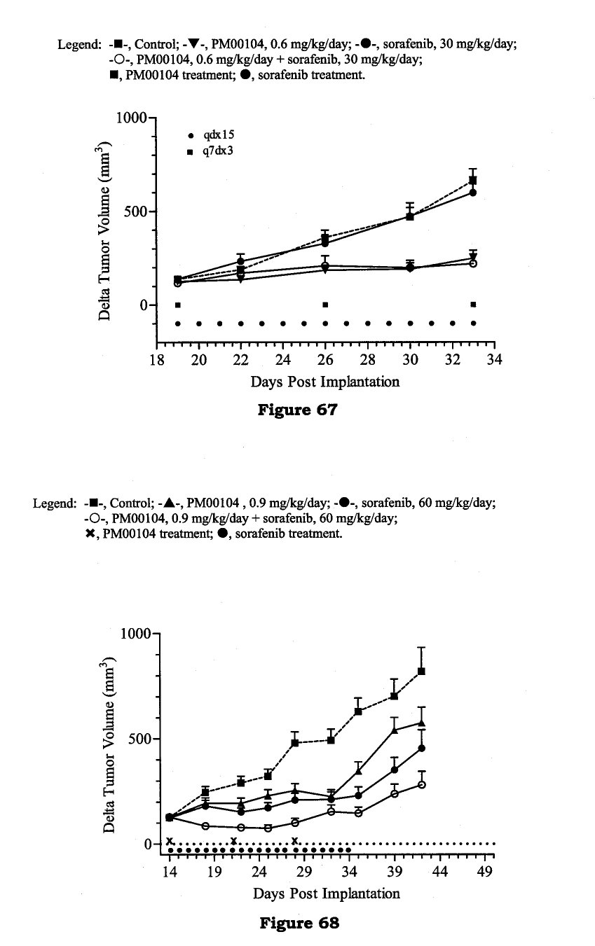

Table 64 reports the % T/C values obtained with each of the treatments and FIG. 64-67 show the tumor volume evaluation (mean±SEM) of HepG2 tumors in mice treated with control (vehicle), PM00104, sorafenib, and the corresponding combinations.

TABLE 64

| | % T/C on day | | | | |
|---|---|---|---|---|---|
| Group | 19 | 22 | 26 | 30 | 33 |
| G1 | — | — | — | — | — |
| (Control group) | | | | | |
| G2 | 91.5 | 59.7 | 40.5 | 23.9 | 27.0 |
| G3 | 91.6 | 71.9 | 51.7 | 40.8 | 37.6 |
| G4 | 88.8 | 78.7 | 60.1 | 61.8 | 66.2 |
| G5 | 101.4 | 123.5 | 91.3 | 100.9 | 90.4 |
| G6 | 96.4 | 75.9 | 41.1 | 33.3 | 19.2 |
| G7 | 97.9 | 83.5 | 43.7 | 32.4 | 22.6 |
| G8 | 94.1 | 97.1 | 55.0 | 46.8 | 30.9 |
| G9 | 85.5 | 90.5 | 58.4 | 42.4 | 33.3 |

Table 65 shows the % of tumor growth inhibition of PM00104 and sorafenib administered as single agents and in combination at a dose of 0.9 mg/kg/day of PM00104 and 60 mg/kg/day of sorafenib. Additionally, the potentiation and the degree of additivity of the combination of PM00104 with sorafenib at said doses are provided.

TABLE 65

| | % Inhibition | | | Expected | Actual | | Degree of |
|---|---|---|---|---|---|---|---|
| Day | G2 | G4 | G6 | Response | Response | Potentiation | Response |
| 19 | 8.5 | 11.2 | 3.6 | 19.7 | −16.1 | no | — |
| 22 | 40.3 | 21.3 | 24.1 | 61.6 | −37.5 | no | — |
| 26 | 59.5 | 39.9 | 58.9 | 99.4 | −40.5 | no | — |
| 30 | 76.1 | 38.2 | 66.7 | 114.3 | −47.6 | no | — |
| 33 | 73.0 | 33.8 | 80.8 | 106.8 | −26.0 | yes | Less than additive |

Table 66 shows the % of tumor growth inhibition of PM00104 and sorafenib administered as single agents and in combination at a dose of 0.9 mg/kg/day of PM00104 and 30 mg/kg/day of sorafenib. Additionally, the potentiation and the degree of additivity of the combination of PM00104 with sorafenib at said doses are provided.

TABLE 66

| | % Inhibition | | | Expected | Actual | Poten- | Degree of |
|---|---|---|---|---|---|---|---|
| Day | G2 | G5 | G7 | Response | Response | tiation | Response |
| 19 | 8.5 | −1.4 | 2.1 | 7.1 | −5.0 | no | — |
| 22 | 40.3 | −23.5 | 16.5 | 16.8 | −0.3 | no | — |
| 26 | 59.5 | 8.7 | 56.3 | 68.2 | −11.9 | no | — |
| 30 | 76.1 | −0.9 | 67.6 | 75.2 | −7.6 | no | — |
| 33 | 73.0 | 9.6 | 77.4 | 82.6 | −5.2 | yes | Additive |

Table 67 shows the % of tumor growth inhibition of PM00104 and sorafenib administered as single agents and in combination at a dose of 0.6 mg/kg/day of PM00104 and 60 mg/kg/day of sorafenib. Additionally, the potentiation and the degree of additivity of the combination of PM00104 with sorafenib at said doses are provided.

TABLE 67

| | % Inhibition | | | Expected | Actual | | Degree of |
|---|---|---|---|---|---|---|---|
| Day | G2 | G4 | G8 | Response | Response | Potentiation | Response |
| 19 | 8.4 | 11.2 | 5.9 | 19.6 | −13.7 | no | — |
| 22 | 28.1 | 21.3 | 2.9 | 49.4 | −46.5 | no | — |
| 26 | 48.3 | 39.9 | 45.0 | 88.2 | −43.2 | no | — |
| 30 | 59.2 | 38.2 | 53.2 | 97.4 | −44.2 | no | — |
| 33 | 62.4 | 33.8 | 69.1 | 96.2 | −27.1 | yes | Less than additive |

Table 68 shows the % of tumor growth inhibition of PM00104 and sorafenib administered as single agents and in combination at a dose of 0.6 mg/kg/day of PM00104 and 30 mg/kg/day of sorafenib. Additionally, the potentiation and the degree of additivity of the combination of PM00104 with sorafenib at said doses are provided.

TABLE 68

| | % Inhibition | | | Expected | Actual | Poten- | Degree of |
|---|---|---|---|---|---|---|---|
| Day | G2 | G5 | G9 | Response | Response | tiation | Response |
| 19 | 8.4 | −1.4 | 14.5 | 7.0 | 7.5 | yes | Additive |
| 22 | 28.1 | −23.5 | 9.5 | 4.6 | 4.9 | no | — |
| 26 | 48.3 | 8.7 | 41.6 | 57.0 | −15.4 | no | — |
| 30 | 59.2 | −0.9 | 57.6 | 58.3 | −0.7 | no | — |
| 33 | 62.4 | 9.6 | 66.7 | 72.0 | −5.3 | yes | Additive |

According to this assay it was found that the combination of PM00104 and sorafenib resulted in a statistically significant (p<0.01) potentiation of antitumor activity over results obtained with Sorafenib control groups. The potentiation observed was graded as less than additive.

### Example 16

In vivo studies to determine the effect of PM00104 in combination with Sorafenib in human hepatoma xenografts.

The aim of these studies was to evaluate the ability of PM00104 to potentiate the antitumor activity of sorafenib by using a xenograft model of human hepatoma.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation with a tumor cell suspension. The Vehicle Control group contained 15 mice and the treated groups had each 10 mice/group.

The tumor model used in these studies was PLC/PRF/5 cell line, which was obtained from the ATCC (Manassas, Va.).

PLC/PRF/5 were grown in Eagle's minimum essential medium supplemented with 10% FBS and 1% L-glutamine. Each animal was implanted SC on the right flank, using a 13G trochar and 1 mL syringe, with 5×10⁶ PLC/PRF/5 cells in a 0.2 mL suspension of 50% Matrigel and 50% serum free MEM medium, without antibiotics. Bacterial cultures were performed on aliquots of the cells prepared for implantation. All cultures were negative for bacterial contamination at both 24 and 48 hours post-implant.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 14.

PM00104 was provided in the form of vials of lyophilized PM00104 powder which was reconstituted with water for injection. Sorafenib was provided in the form of a tablet which was dissolved in Cremophor EL/ethanol/water (CEW) (12.5, 12.5, 75) final proportion.

Study groups and treatment regimens are listed in table 69.

TABLE 69

| Group | Dose | Route | Schedule | Test material |
|---|---|---|---|---|
| G1 | 10 ml/kg/day | IV | A | 0.18% Placebo in Saline |
| (Control | 10 ml/kg/day | PO | B | 12.5/12.5/75 CEW |
| group) | | | | |
| G2 | 0.90 mg/kg/day | IV | A | PM00104 |
| G3 | 0.45 mg/kg/day | IV | A | PM00104 |
| G4 | 60 mg/kg/day | PO | B | Sorafenib |
| G5 | 30 mg/kg/day | PO | B | Sorafenib |
| G6 | 0.90 mg/kg/day | IV | A | PM00104 |
| | 60 mg/kg/day | PO | B | Sorafenib |
| G7 | 0.90 mg/kg/day | IV | A | PM00104 |
| | 30 mg/kg/day | PO | B | Sorafenib |
| G8 | 0.45 mg/kg/day | IV | A | PM00104 |
| | 60 mg/kg/day | PO | B | Sorafenib |
| G9 | 0.45 mg/kg/day | IV | A | PM00104 |
| | 30 mg/kg/day | PO | B | Sorafenib |

A: DPI 14, 21 and 28;
B: DPI 14-34
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and sorafenib) against sorafenib mean tumor weight, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

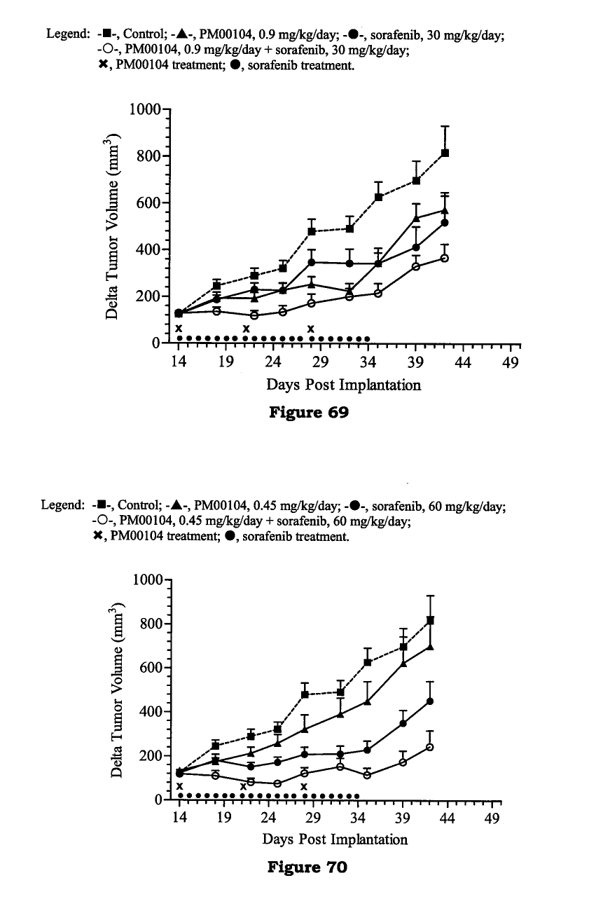

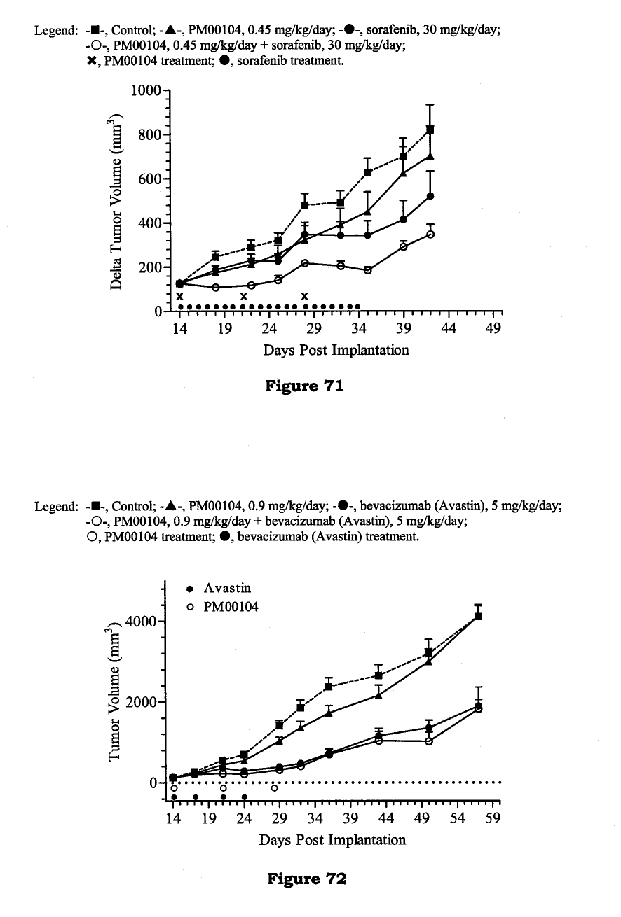

Table 70 reports the % T/C values obtained with each of the treatments and FIG. 68-71 show the tumor volume evaluation (mean±SEM) of PLC/PRF/5 tumors in mice treated with control (vehicle), PM00104, sorafenib, and the corresponding combinations.

TABLE 70

| | % T/C on day | | | | | | | | |
|---|---|---|---|---|---|---|---|---|---|
| Group | 14 | 18 | 22 | 25 | 28 | 32 | 35 | 39 | 42 |
| G1 | — | — | — | — | — | — | — | — | — |
| (Control | | | | | | | | | |
| group) | | | | | | | | | |
| G2 | 100.8 | 78.8 | 66.7 | 71.0 | 52.9 | 45.6 | 54.9 | 77.1 | 70.1 |
| G3 | 106.5 | 71.1 | 73.5 | 80.4 | 67.3 | 79.7 | 71.7 | 89.2 | 85.7 |
| G4 | 98.5 | 73.6 | 52.2 | 53.4 | 43.4 | 42.9 | 36.5 | 50.2 | 55.4 |
| G5 | 99.5 | 76.1 | 79.5 | 70.7 | 72.4 | 69.9 | 54.7 | 59.3 | 72.0 |
| G6 | 101.7 | 34.7 | 27.0 | 23.3 | 20.9 | 31.2 | 23.2 | 33.9 | 34.3 |
| G7 | 102.9 | 55.5 | 41.0 | 41.7 | 36.0 | 40.9 | 34.4 | 47.4 | 45.0 |
| G8 | 95.4 | 45.1 | 28.5 | 23.5 | 25.6 | 31.3 | 18.4 | 24.9 | 29.8 |
| G9 | 100.3 | 43.9 | 40.8 | 43.6 | 45.3 | 41.5 | 29.3 | 41.7 | 42.4 |

Table 71 shows the % of tumor growth inhibition of PM00104 and sorafenib administered as single agents and in combination at a dose of 0.9 mg/kg/day of PM00104 and 60 mg/kg/day of sorafenib. Additionally, the potentiation and the degree of additivity of the combination of PM00104 with sorafenib at said doses are provided.

TABLE 71

| | % Inhibition | | | Expected | Actual | | Degree of |
|---|---|---|---|---|---|---|---|
| Day | G2 | G4 | G6 | Response | Response | Potentiation | Response |
| 14 | −0.8 | 1.5 | −1.7 | 0.7 | −2.4 | no | — |
| 18 | 21.2 | 26.4 | 65.3 | 47.6 | 17.7 | yes | Greater than additive |
| 22 | 33.3 | 47.8 | 73.0 | 81.1 | −8.1 | yes | Additive |
| 25 | 29.0 | 46.6 | 76.7 | 75.6 | 1.1 | yes | Additive |
| 28 | 47.1 | 56.6 | 79.1 | 103.7 | −24.6 | yes | Less than additive |
| 32 | 54.4 | 57.1 | 68.8 | 111.5 | −42.7 | yes | Less than additive |
| 35 | 45.1 | 63.5 | 76.8 | 108.6 | −31.8 | yes | Less than additive |
| 39 | 22.9 | 49.8 | 66.1 | 72.7 | −6.6 | yes | Additive |
| 42 | 29.9 | 44.6 | 65.7 | 74.5 | −8.8 | yes | Additive |

Table 72 shows the % of tumor growth inhibition of PM00104 and sorafenib administered as single agents and in combination at a dose of 0.9 mg/kg/day of PM00104 and 30 mg/kg/day of sorafenib. Additionally, the potentiation and the degree of additivity of the combination of PM00104 with sorafenib at said doses are provided.

TABLE 72

| | % Inhibition | | | Expected | Actual | | Degree of |
|---|---|---|---|---|---|---|---|
| Day | G2 | G5 | G7 | Response | Response | Potentiation | Response |
| 14 | −0.8 | 0.5 | −2.9 | −0.3 | −2.6 | no | — |
| 18 | 21.2 | 23.9 | 44.5 | 45.1 | −0.6 | yes | Additive |
| 22 | 33.3 | 20.5 | 59.0 | 53.8 | 5.2 | yes | Additive |
| 25 | 29.0 | 29.3 | 58.3 | 58.3 | 0.0 | yes | Additive |
| 28 | 47.1 | 27.6 | 64.0 | 74.7 | −10.7 | yes | Additive |
| 32 | 54.4 | 30.1 | 59.1 | 84.5 | −25.4 | yes | Less than additive |
| 35 | 45.1 | 45.3 | 65.6 | 90.4 | −24.8 | yes | Less than additive |
| 39 | 22.9 | 40.7 | 52.6 | 63.6 | −11.0 | yes | Less than additive |
| 42 | 29.9 | 28.0 | 55.0 | 57.9 | −2.9 | yes | Additive |

Table 73 shows the % of tumor growth inhibition of PM00104 and sorafenib administered as single agents and in combination at a dose of 0.45 mg/kg/day of PM00104 and 60 mg/kg/day of sorafenib. Additionally, the potentiation and the degree of additivity of the combination of PM00104 with sorafenib at said doses are provided.

TABLE 73

| | % Inhibition | | | Expected | Actual | | Degree of |
|---|---|---|---|---|---|---|---|
| Day | G2 | G4 | G8 | Response | Response | Potentiation | Response |
| 14 | −6.5 | 1.5 | 4.6 | −5.0 | 9.6 | no | — |
| 18 | 28.9 | 26.4 | 54.9 | 55.3 | −0.4 | yes | Additive |
| 22 | 26.5 | 47.8 | 71.5 | 74.3 | −2.8 | yes | Additive |
| 25 | 19.6 | 46.6 | 76.5 | 66.2 | 10.3 | yes | Greater than additive |
| 28 | 32.7 | 56.6 | 74.4 | 89.3 | −14.9 | yes | Less than additive |
| 32 | 20.3 | 57.1 | 68.7 | 77.4 | −8.7 | yes | Additive |
| 35 | 28.3 | 63.5 | 81.6 | 91.8 | −10.2 | yes | Additive |
| 39 | 10.8 | 49.8 | 75.1 | 60.6 | 14.5 | yes | Greater than additive |
| 42 | 14.3 | 44.6 | 70.2 | 58.9 | 11.3 | yes | Greater than additive |

Table 74 shows the % of tumor growth inhibition of PM00104 and sorafenib administered as single agents and in combination at a dose of 0.45 mg/kg/day of PM00104 and 30 mg/kg/day of sorafenib. Additionally, the potentiation and the degree of additivity of the combination of PM00104 with sorafenib at said doses are provided.

TABLE 74

| | % Inhibition | | | Expected | Actual | | Degree of |
|---|---|---|---|---|---|---|---|
| Day | G2 | G5 | G9 | Response | Response | Potentiation | Response |
| 14 | −6.5 | 0.5 | −1.5 | −6.0 | 4.5 | no | — |
| 18 | 28.9 | 23.9 | 56.9 | 52.8 | 4.1 | yes | Additive |
| 22 | 26.5 | 20.5 | 59.2 | 47.0 | 12.2 | yes | Greater than additive |
| 25 | 19.6 | 29.3 | 56.4 | 48.9 | 7.5 | yes | Additive |
| 28 | 32.7 | 27.6 | 54.7 | 60.3 | −5.6 | yes | Additive |
| 32 | 20.3 | 30.1 | 58.5 | 50.4 | 8.1 | yes | Additive |
| 35 | 28.3 | 45.3 | 70.7 | 73.6 | −2.9 | yes | Additive |
| 39 | 10.8 | 40.7 | 58.3 | 51.5 | 6.8 | yes | Additive |
| 42 | 14.3 | 28.0 | 57.6 | 42.3 | 15.3 | yes | Greater than additive |

According to this assay it was found that the combination of PM00104 and sorafenib resulted in a statistically significant (p<0.01) potentiation of antitumor activity over results obtained with sorafenib control groups, being the best results observed at the lower doses of both drugs. This best potentiation was graded as greater than additive.

### Example 17

In vivo studies to determine the effect of PM00104 in combination with bevacizumab in human ovarian tumor xenografts.

The aim of these studies was to evaluate the ability of PM00104 to potentiate the antitumor activity of bevacizumab by using a xenograft model of human ovarian cancer.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation with tumor fragments. The Vehicle Control group contained 15 mice and the treated groups had each 10 mice/group.

The tumor model used in these studies was SK-OV-3 cell line, which was obtained from the ATCC (Manassas, Va.).

SK-OV-3 fragments were removed from donor animals and tissue was debrided of membrane and any hemorrhagic and necrotic areas and 3-4 mm³ fragments, from in vivo passage 2, were implanted SC on the right flank of each animal, using a 13G trochar. Bacterial culture was taken on cells used to implant the study. All cultures were negative for bacterial contamination at both 24 and 48 hours post-implant.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 14.

PM00104 was provided in the form of vials of lyophilized PM00104 powder which was reconstituted with water for injection. Bevacizumab was provided as a solution which was further diluted with 0.9% Saline.

Study groups and treatment regimens are listed in table 75.

TABLE 75

| Group | Dose | Route | Schedule | Test material |
|---|---|---|---|---|
| G1 | 10 ml/kg/day | IV | A | 0.18% Placebo |
| (Control group) | 10 ml/kg/day | IP | B | 0.9% Saline |
| G2 | 0.90 mg/kg/day | IV | A | PM00104 |
| G3 | 5 mg/kg/day | IP | B | Bevacizumab |
| G4 | 2.5 mg/kg/day | IP | B | Bevacizumab |
| G5 | 0.90 mg/kg/day | IV | A | PM00104 |
| | 5 mg/kg/day | IP | B | Bevacizumab |
| G6 | 0.90 mg/kg/day | IV | A | PM00104 |
| | 2.5 mg/kg/day | IP | B | Bevacizumab |

A: DPI 14, 21, and 28;
B: DPI 14, 17, 21, 24, 28, and 31
Placebo: 500 mg Sucrose + 34 mg Potassium Phosphate + Phosphoric acid q.s. pH 3.8-4.4

Tumor size measurements were recorded twice weekly from the treatment initiation until the termination of the study. Tumor growth inhibition was assessed comparing the mean tumor weight between the two agents in combination (PM00104 and bevacizumab) against bevacizumab mean tumor weight, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

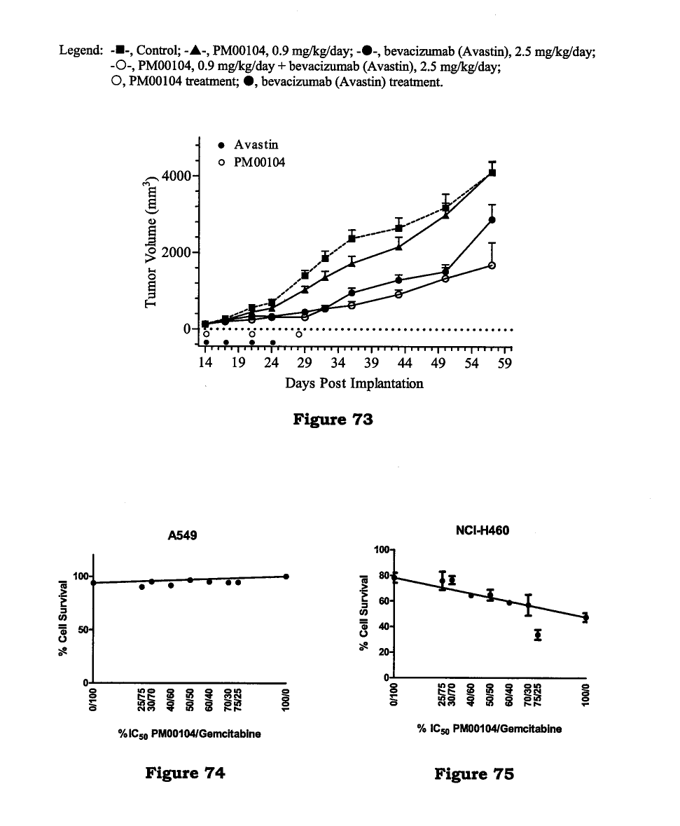

Table 76 reports the % T/C values obtained with each of the treatments and FIG. 72-73 show the tumor volume evaluation (mean±SEM) of SK-OV-3 tumors in mice treated with control (vehicle), PM00104, bevacizumab, and the corresponding combinations.

TABLE 76

| | % T/C on day | | | | | | | | | |
|---|---|---|---|---|---|---|---|---|---|---|
| Group | 14 | 17 | 21 | 24 | 29 | 32 | 36 | 43 | 50 | 57 |
| G1 (Control | — | — | — | — | — | — | — | — | | |
| group) | | | | | | | | | | |
| G2 | 100.9 | 87.6 | 79.4 | 78.8 | 73.5 | 73.1 | 72.7 | 81.5 | 93.8 | 100.4 |
| G3 | 100.2 | 76.6 | 64.0 | 42.6 | 28.0 | 25.8 | 30.7 | 43.8 | 42.5 | 46.1 |
| G4 | 100.5 | 85.0 | 61.0 | 48.4 | 32.1 | 29.6 | 40.3 | 48.7 | 47.5 | 70.0 |
| G5 | 100.9 | 78.1 | 40.5 | 31.2 | 22.6 | 22.2 | 29.5 | 39.2 | 32.0 | 44.3 |
| G6 | 103.4 | 73.0 | 43.8 | 45.2 | 22.4 | 29.3 | 26.3 | 34.6 | 42.0 | 41.0 |

Table 77 shows the % of tumor growth inhibition of PM00104 and bevacizumab administered as single agents and in combination at a dose of 0.9 mg/kg/day of PM00104 and 5 mg/kg/day of bevacizumab. Additionally, the potentiation and the degree of additivity of the combination of PM00104 with bevacizumab at said doses are provided.

TABLE 77

| | % Inhibition | | | Expected | Actual | | Degree of |
|---|---|---|---|---|---|---|---|
| Day | G2 | G3 | G5 | Response | Response | Potentiation | Response |
| 14 | −0.9 | −0.2 | −0.9 | −1.1 | 0.2 | no | — |
| 17 | 12.4 | 23.4 | 21.9 | 35.8 | −13.9 | yes | Less than additive |
| 21 | 20.6 | 36.0 | 59.5 | 56.6 | 2.9 | yes | Additive |
| 24 | 21.2 | 57.4 | 68.8 | 78.6 | −9.8 | yes | Additive |
| 29 | 26.5 | 72.0 | 77.4 | 98.5 | −21.1 | yes | Less than additive |
| 32 | 26.9 | 74.2 | 77.8 | 101.1 | −23.3 | yes | Less than additive |
| 36 | 27.3 | 69.3 | 70.5 | 96.6 | −26.1 | yes | Less than additive |
| 43 | 18.5 | 56.2 | 60.8 | 74.7 | −13.9 | yes | Less than additive |
| 50 | 6.2 | 57.5 | 68.0 | 63.7 | 4.3 | yes | Additive |
| 57 | −0.4 | 53.9 | 55.7 | 53.5 | 2.2 | yes | Additive |

Table 78 shows the % of tumor growth inhibition of PM00104 and bevacizumab administered as single agents and in combination at a dose of 0.9 mg/kg/day of PM00104 and 2.5 mg/kg/day of bevacizumab. Additionally, the potentiation and the degree of additivity of the combination of PM00104 with bevacizumab at said doses are provided.

TABLE 78

| | % Inhibition | | | Expected | Actual | | Degree of |
|---|---|---|---|---|---|---|---|
| Day | G2 | G4 | G6 | Response | Response | Potentiation | Response |
| 14 | −0.9 | −0.5 | −3.4 | −1.4 | −2.0 | no | — |
| 17 | 12.4 | 15.0 | 27.0 | 27.4 | −0.4 | yes | Additive |
| 21 | 20.6 | 39.0 | 56.2 | 59.6 | −3.4 | yes | Additive |
| 24 | 21.2 | 51.6 | 54.8 | 72.8 | −18.0 | yes | Less than additive |
| 29 | 26.5 | 67.9 | 77.6 | 94.4 | −16.8 | yes | Less than additive |
| 32 | 26.9 | 70.4 | 70.7 | 97.3 | −26.6 | yes | Less than additive |
| 36 | 27.3 | 59.7 | 73.7 | 87.0 | −13.3 | yes | Less than additive |
| 43 | 18.5 | 51.3 | 65.4 | 69.8 | −4.4 | yes | Additive |
| 50 | 6.2 | 52.5 | 58.0 | 58.7 | −0.7 | yes | Additive |
| 57 | −0.4 | 30.0 | 59.0 | 29.6 | 29.4 | yes | Greater than additive |

According to this assay it was found that the combination of PM00104 and bevacizumab resulted in potentiation of antitumor activity over results obtained with either of the single agent control groups. At the lower bevacizumab dose this potentiation was graded as greater than additive at the end of the assay.

### Example 18

In vitro studies to determine the effect of PM00104 in combination with chemotherapeutic agents on human lung, breast and colon cancer cell lines.

The objective of this study was to determine the ability of PM00104 to potentiate the antitumor activity of chemotherapeutic agents used in the treatment of lung, breast and colon cancer.

The following agents were evaluated in combination with PM00104: paclitaxel, cisplatin, gemcitabine, doxorubicin, 5-fluorouracil (5-FU), irinotecan, and oxaliplatin. The human cancer cell lines selected for this assay were the following: A-549 (lung cancer), NCI-H460 (lung cancer), NCI-H23 (lung cancer), MDA-MB-231 (breast cancer), BT-474 (breast cancer), MCF-7 (breast cancer), LoVo (colon cancer), HCT-116 (colon cancer), and HT-29 (colon cancer) cell lines.

A-549, NCI-H460, MDA-MB-231, MCF-7, LoVo and HT-29 cells were grown in Dulbecco's Modified Eagle's Medium (DMEM) supplemented with 10% Fetal Bovine Serum (FBS), 1% penicillin/streptomycin and 2 mM L-glutamine.

NCI-H23 cells were grown in RPMI supplemented with 10% Fetal Bovine Serum (FBS), 1% penicillin/streptomycin and 2 mM L-glutamine.

BT-474 cells were grown in RPMI supplemented with 1% ITS (insulin, transferrin and selenium), 10% Fetal Bovine Serum (FBS), 1% penicillin/streptomycin and 2 mM L-glutamine.

HCT-116 cells were grown in McCoy's supplemented with 10% Fetal Bovine Serum (FBS), 1% penicillin/streptomycin and 2 mM L-glutamine.

The screening was performed in two parts:

a. In the first set of assays, IC₅₀ values were determined for each drug after 72 hours of drug exposure in each of the tumor cell lines.

All cell lines were maintained in their respective growth medium at 37° C., 5% CO₂ and 98% humidity. Before plating, cell cultures were trypsinized and cell number estimated using an automated flow cytometer.

Cells were harvested and seeded in 96 well microtiter plates at the appropriate cell density (5,000-10,000 cells) in 150 μL of media and incubated for 24 hours to allow the cells to attach before drug addition.

Stock solutions of PM00104, paclitaxel, gemcitabine, doxorubicin, 5-fluorouracil (5-FU) and irinotecan were prepared in 100% DMSO at 1.0 mg/mL. Stock solutions of cisplatin and oxaliplatin were prepared in double sterile water for tissue culture at 1.0 mg/mL. Additional serial dilutions were prepared in serum-free culture medium to achieve a final 4× treatment concentration. 50 μL of each diluted test articles was added per well.

The cytotoxic effect was measured by the MIT Assay (Tetrazolium), which is a colorimetric method for determining the number of viable cells. After the incubation period (72 hours), 50 μL of MIT solution was added to each microtiter well and incubated for further 8 hours at 37° C. The culture medium was then removed and 50 μl, of DMSO were added to dissolve the MIT crystals. Optical densities were read at 540 nm on spectrophotometer microplate reader.

IC₅₀ values were calculated from an average of two to four assays for each of the test agents. A regression curve was generated using Prism v5.02 software (GraphPad) and then 50% inhibition concentration was automatically interpolated.

The individual IC₅₀ values of each agent for each cell line are shown in table 79.

TABLE 79
IC₅₀ values (Molar) for each of the agent

| Cell Line | Compound | IC₅₀ |
|---|---|---|
| A-549 | PM00104 | 7.2E−09 |
| | Gemcitabine | 5.0E−10 |
| | Paclitaxel | 4.7E−08 |
| | Cisplatin | 5.0E−05 |
| NCI-H460 | PM00104 | 7.2E−09 |
| | Gemcitabine | 1.0E−09 |
| | Paclitaxel | 1.2E−08 |
| | Cisplatin | 2.5E−05 |
| NCI-H23 | PM00104 | 5.0E−09 |
| | Gemcitabine | 2.8E−10 |
| | Paclitaxel | 4.7E−08 |
| | Cisplatin | 1.0E−05 |
| MDA-MB-231 | PM00104 | 6.0E−09 |
| | Gemcitabine | 2.4E−08 |
| | Paclitaxel | 6.0E−09 |
| | Doxorubicin | 1.9E−06 |
| BT-474 | PM00104 | 1.0E−09 |
| | Gemcitabine | 1.4E−08 |
| | Paclitaxel | 1.7E−09 |
| | Doxorubicin | 2.5E−06 |
| MCF-7 | PM00104 | 1.0E−09 |
| | Gemcitabine | 1.3E−09 |
| | Paclitaxel | 7.0E−08 |
| | Doxorubicin | 4.0E−06 |
| HCT-116 | PM00104 | 9.0E−09 |
| | 5-Fluorouracil | 6.0E−06 |
| | Oxaliplatin | 1.4E−04 |
| | Irinotecan | 8.0E−06 |
| LOVO | PM00104 | 3.0E−09 |
| | 5-Fluorouracil | 1.8E−06 |
| | Oxaliplatin | 3.2E−06 |
| | Irinotecan | 6.0E−06 |
| HT-29 | PM00104 | 6.0E−09 |
| | 5-Fluorouracil | 4.0E−06 |
| | Oxaliplatin | 3.8E−05 |
| | Irinotecan | 1.8E−05 |

b. In a second set of assays, the cell lines were incubated with PM00104 in combination with each of the agents mentioned above in the following combination of unique IC₅₀ concentrations:

| IC₅₀ of PM00104 | IC₅₀ of Agent |
|---|---|
| 100% | 0% |
| 75% | 25% |
| 70% | 30% |
| 60% | 40% |
| 50% | 50% |
| 40% | 60% |
| 30% | 70% |
| 25% | 75% |
| 0% | 100% |

Cell culture and cell plating were performed as described before. Stock solutions of each drug were also prepared as described before at a drug concentration of 1.0 mg/mL. These stock solutions were serially diluted further as needed to reach the starting concentration. Additional serial dilutions were prepared in serum-free culture medium to achieve a final 8× treatment concentration. 25 μL of each diluted test articles was added per well.

The cytotoxic effect was measured by the MIT Assay as described above. Data was analyzed as follows:

1. Prism v5.02 software (Graphpad) program was used to normalize the data to control values (100%=cell growth in the absence of agent (vehicle alone); 0%=blank control).
2. Normalized data were plotted as x/y graphs. A line was drawn connecting the values of 100% ICₛₒ for each agent (drug). Values significantly above the line indicated antagonism, significantly below indicated synergy, and on the line indicated additivity.

Synergistic cytotoxicity to tumor cells is an optimal effect and implies that the combination of PM00104 with another drug is more effective than either drug alone. A statistically significant observation requires that a difference exists between the combination (PM00104+another drug) % cell survival value and both endpoint values (PM00104 and the other drug alone). If the majority of the values are statistically above or below the line (endpoints) then antagonism or synergy is described, respectively, otherwise the pattern is more consistent with an additive interaction.

According to this assay it was found that:

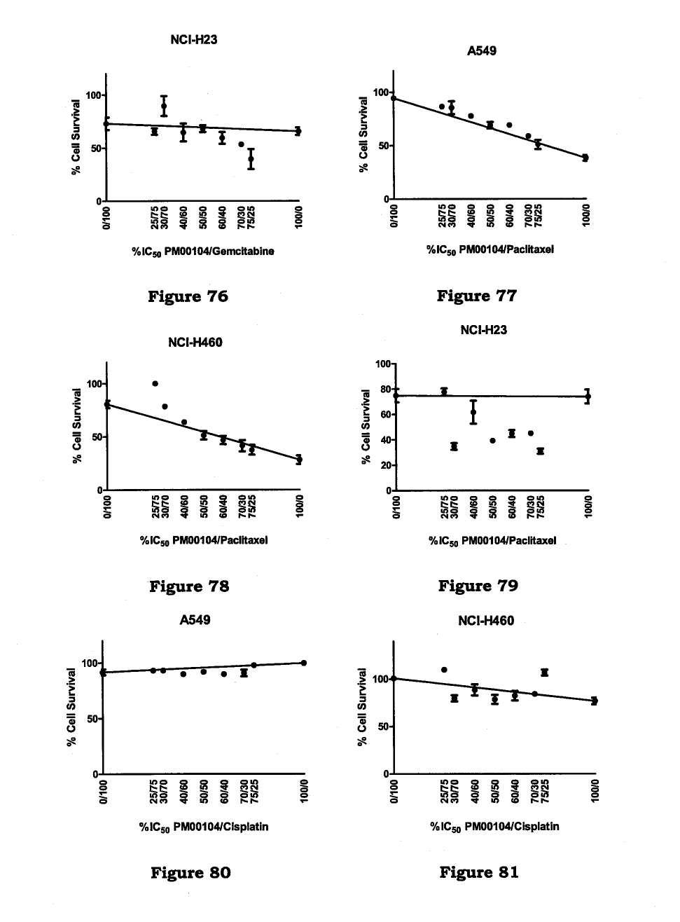

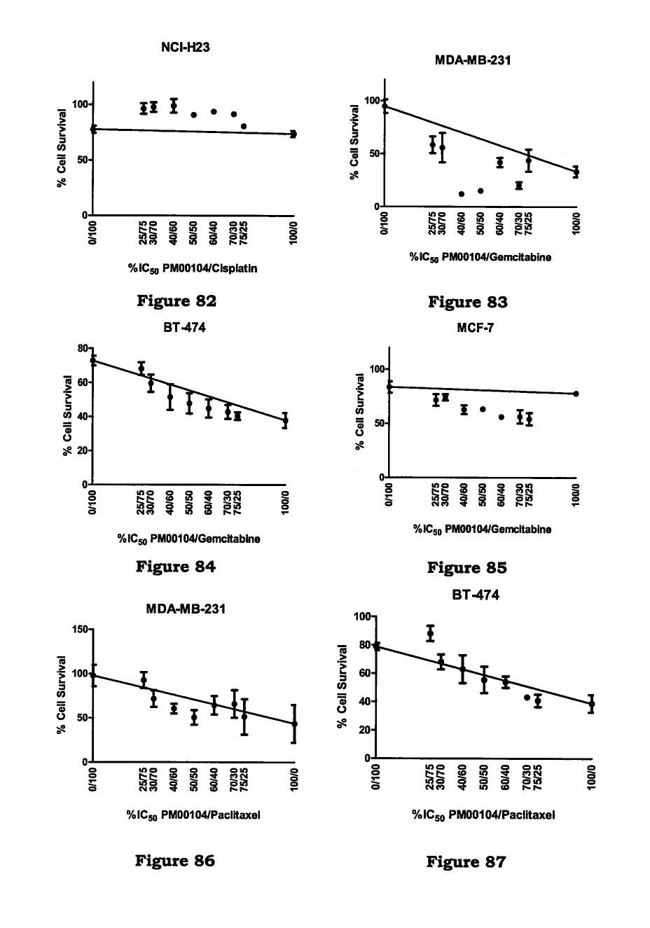

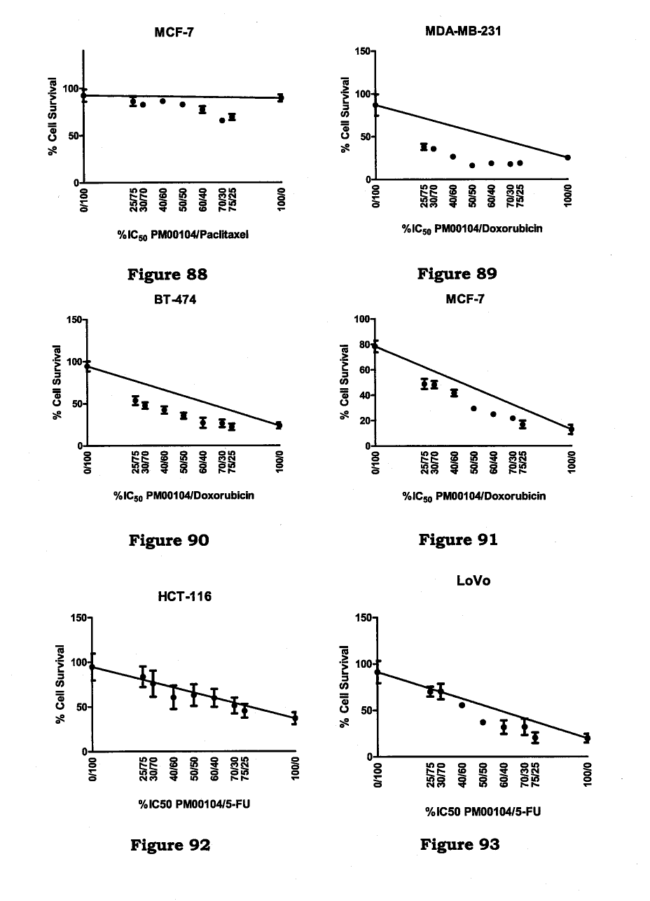

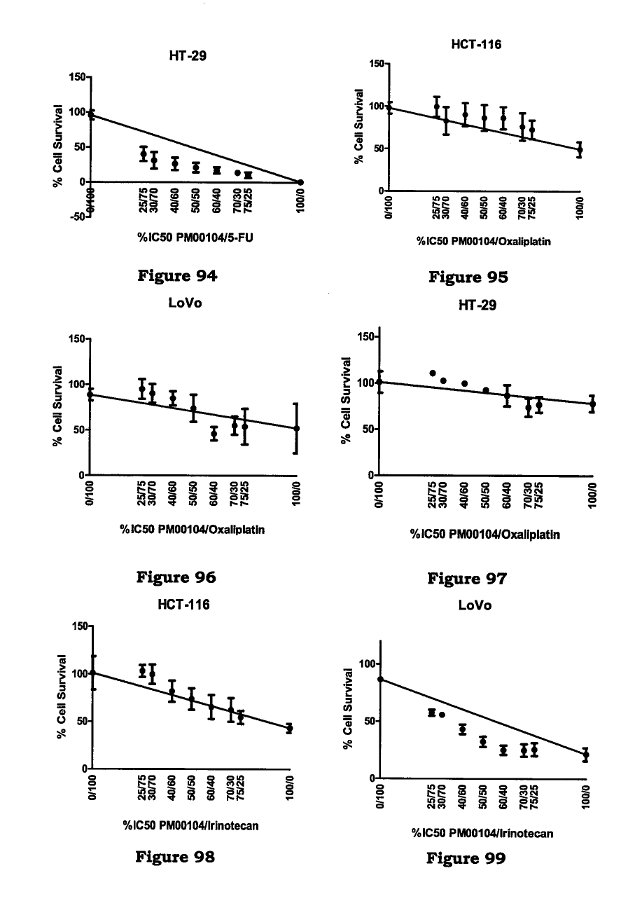

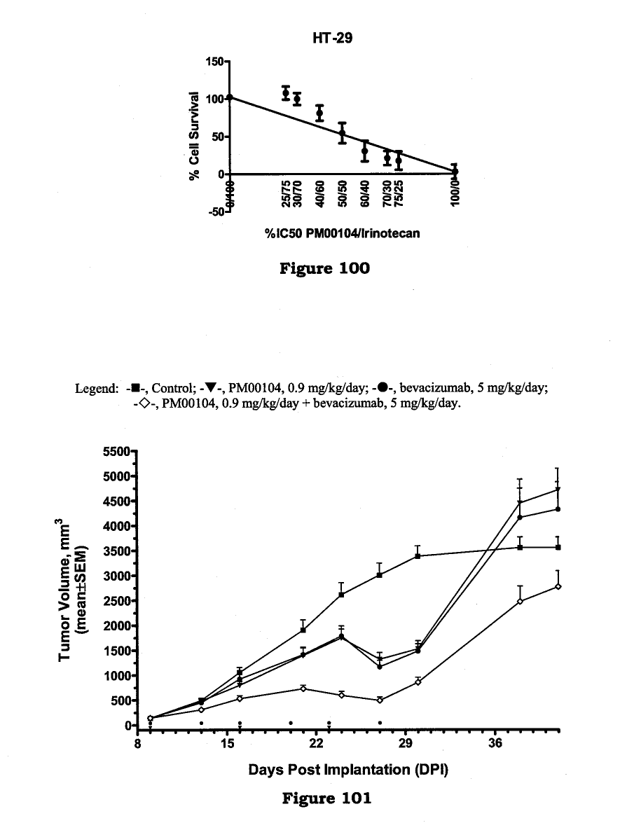

a. The combination of PM00104 with gemcitabine in human lung cancer cells was synergistic in NCI-H23 (FIG. 76) cell line at 60/40, 70/30 and 75/25 dose ratios and in NCI-H460 (FIG. 75) cell line it showed an additive trend having a synergistic effect at 75/25 dose ratio. Additionally, in A549 (FIG. 74) cell line the combination showed an additive trend.
b. The combination of PM00104 with paclitaxel in human lung cancer cells was synergistic in NCI-H23 (FIG. 79) cell line, and showed an additive trend in NCI-H460 (FIG. 78) and A549 (FIG. 77) cell lines.
c. The combination of PM00104 with cisplatin in human lung cancer cells was synergistic in NCI-H460 (FIG. 81) cell line at 30/70 and 50/50 dose ratios. In A549 (FIG. 80) cell line showed an additive trend and in NCI-H23 (FIG. 82) cell line an antagonistic trend.
d. The combination of PM00104 with gemcitabine in human breast cancer cells was synergistic in MDA-MB-231 (FIG. 83), BT-474 (FIG. 84) and MCF-7 (FIG. 85) cell lines.
e. The combination of PM00104 with paclitaxel in human breast cancer cells was synergistic in MCF-7 (FIG. 88) cell line and it showed an additive trend in MDA-MB-231 (FIG. 86) and BT-474 (FIG. 87) cell lines.
f. The combination of PM00104 with doxorubicin in human breast cancer cells was synergistic in MDA-MB-231 (FIG. 89), BT-474 (FIG. 90) and MCF-7 (FIG. 91) cell lines.
g. The combination of PM00104 with 5-fluorouracil in colon cancer cells was synergistic in HT-29 (FIG. 94) and LoVo (FIG. 93) cell lines at all or almost all dose ratios, and it showed an additive trend in HCT-116 (FIG. 92).
h. The combination of PM00104 with oxaliplatin in human colon cancer cells showed an additive trend in LoVo (FIG. 96), HT-29 (FIG. 97) and HCT-116 (FIG. 95) cell lines.
i. The combination of PM00104 with irinotecan in human colon cancer cells was synergistic in LoVo (FIG. 99) cell line and it showed an additive trend in HT-29 (FIG. 100) and HCT-116 (FIG. 98) cell lines.

### Example 19

In vivo studies to determine the effect of PM00104 in combination with temsirolimus and bevacizumab in human lung cancer xenografts.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation. The Vehicle Control group contained 15 mice and the treated groups had each 10 mice/group.

The tumor model used in this study was NCI-H460 cell line which is a human NSCLC cell line obtained from the ATCC (Manassas, Va.). NCI-H460 cells were grown in RPMI-1640 medium, 10% FBS, 10 mM Hepes, 1 mM sodium pyruvate, 4.5 g/l glucose, 1.5 g/l sodium bicarbonate and 2 mM L-glutamine. Cells from in vitro passage 7 were implanted SC into study mice using a 1 ml syringe with a 13G trocar: 5×10⁶ cells/mouse in 0.2 ml 50% Matrigel/50% RPMI medium of NCI-H460 without serum or antibiotics. Bacterial culture was taken on cells used to implant the study. All cultures were negative for bacterial contamination at both 24 and 48 hours post-implant.

Tumor measurements were determined as disclosed in Example 4. When tumors reached an appropriated volume, within the size range of 139±36 mm³ (mean±SD), mice were randomized into the treatment and control groups based on tumor weight by using LabCat® In Life module V 8.0 SP1 tumor tracking and measurement software. Treatments were initiated on DPI 9.

PM00104 was provided in the form of vials of lyophilized PM00104 powder which was reconstituted with water for injection. Temsirolimus was provided in the form of a non-aqueous ethanolic solution which was further diluted with a diluent solution containing polysorbate 80 (40% w/v), polyethylene glycol 400 (42.8% w/v) and dehydrated alcohol (19.9% w/v) and, then, further diluted in 0.9% saline to the dosing concentrations. Bevacizumab was provided as a solution which was further diluted with 0.9% saline.

Study groups and treatment regimens are listed in table 80.

TABLE 80

| Group | Dose | Route | Schedule | Test material |
|---|---|---|---|---|
| G1 | 10 ml/kg/day | IV | A | 0.18% Placebo |
| (Control group) | 10 ml/kg/day | IP | B | 0.9% Saline |
| G2 | 0.9 mg/kg/day | IV | A | PM00104 |
| G3 | 5 mg/kg/day | IP | C | Bevacizumab |
| G4 | 2.5 mg/kg/day | IP | C | Bevacizumab |
| G5 | 20 mg/kg/day | IP | B | Temsirolimus |
| G6 | 10 mg/kg/day | IP | B | Temsirolimus |
| G7 | 0.9 mg/kg/day | IV | A | PM00104 |
| | 5 mg/kg/day | IP | C | Bevacizumab |
| G8 | 0.9 mg/kg/day | IV | A | PM00104 |
| | 2.5 mg/kg/day | IP | C | Bevacizumab |
| G9 | 0.9 mg/kg/day | IV | A | PM00104 |
| | 20 mg/kg/day | IP | B | Temsirolimus |
| G10 | 0.9 mg/kg/day | IV | A | PM00104 |
| | 10 mg/kg/day | IP | B | Temsirolimus |

A: DPI 9, 16, and 23;
B: DPI 9-13, 16-20, and 23-27;
C: DPI 9, 13, 16, 20, 23, and 27
Placebo: 500 mg Sucrose + 34 mg Potassium Phosphate + Phosphoric acid q.s. pH 3.8-4.4

Tumor size measurements were recorded 2-3 times/week from the treatment initiation until the termination of the study. Tumor growth inhibition was assessed comparing the mean tumor weight between the two agents in combination (PM00104 and bevacizumab or PM00104 and temsirolimus) against bevacizumab or temsirolimus mean tumor weight, respectively, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

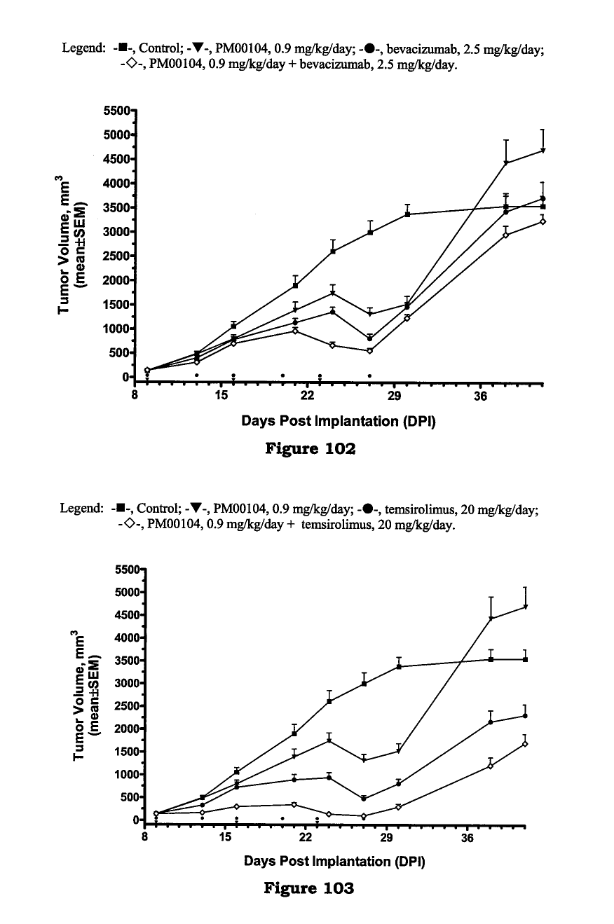

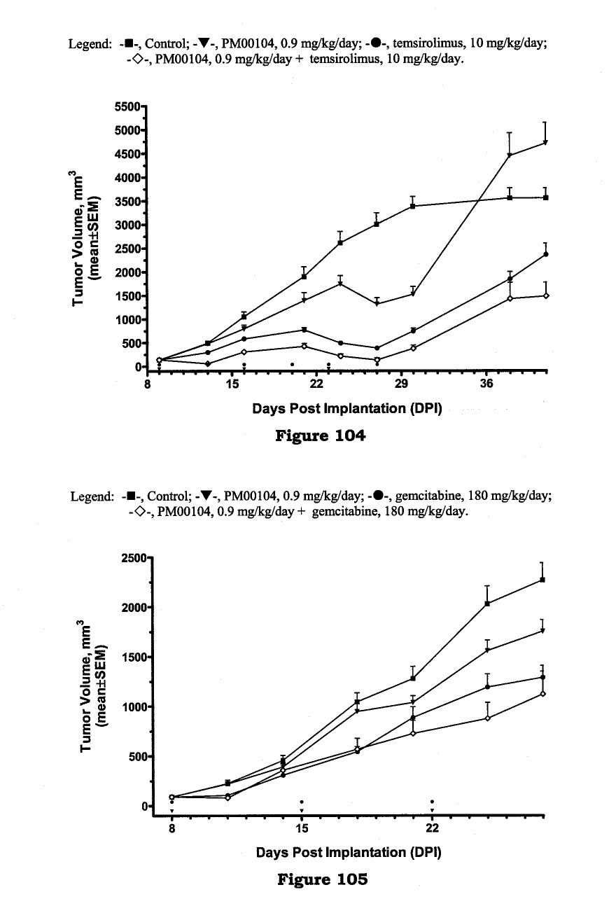

Table 81 reports the % T/C values obtained with each of the treatments and FIGS. 101-104 show the tumor volume evaluation (mean±SEM) of NCI-H460 tumors in mice treated with control, PM00104, bevacizumab, temsirolimus and the corresponding combinations.

TABLE 81

| | % T/C on day | | | | | | | | |
|---|---|---|---|---|---|---|---|---|---|
| Group | 9 | 13 | 16 | 21 | 24 | 27 | 30 | 38 | 41 |
| G1 | — | — | — | — | — | — | — | — | |
| G2 | 96.3 | 97.8 | 75.9 | 73.2 | 66.8 | 43.8 | 45.1 | 125.1 | 132.5 |
| G3 | 102.1 | 91.3 | 87.6 | 74.2 | 68.3 | 38.7 | 43.7 | 116.9 | 121.6 |
| G4 | 99.4 | 81.7 | 74.3 | 59.7 | 52.0 | 26.9 | 43.3 | 96.8 | 104.7 |
| G5 | 104.4 | 66.4 | 68.4 | 46.8 | 35.9 | 15.9 | 23.9 | 61.2 | 65.2 |
| G6 | 101.9 | 60.4 | 55.5 | 40.5 | 18.9 | 12.9 | 22.0 | 52.0 | 66.4 |
| G7 | 104.1 | 62.3 | 50.2 | 38.2 | 22.7 | 16.4 | 25.2 | 69.5 | 77.9 |
| G8 | 105.5 | 63.1 | 66.4 | 50.5 | 25.6 | 18.5 | 36.5 | 83.4 | 91.4 |
| G9 | 100.5 | 33.0 | 28.2 | 18.0 | 5.3 | 3.4 | 8.8 | 34.0 | 47.9 |
| G10 | 103.0 | 11.7 | 29.2 | 22.3 | 8.3 | 4.5 | 11.2 | 40.1 | 41.7 |

Table 82 shows the % of tumor growth inhibition of PM00104 and bevacizumab administered as single agents and in combination at a dose of 0.9 mg/kg/day of PM00104 and 5 mg/kg/day of bevacizumab. Additionally, the potentiation and the degree of additivity of the combination of PM00104 with bevacizumab at said doses are provided.

TABLE 82

| | % Inhibition | | | Expected | Actual | | Degree of |
|---|---|---|---|---|---|---|---|
| Day | G2 | G3 | G7 | Response | Response | Potentiation | Response |
| 9 | 3.7 | −2.1 | −4.1 | 1.6 | −5.7 | no | — |
| 13 | 2.2 | 8.7 | 37.7 | 10.9 | 26.8 | yes | Greater than additive |
| 16 | 24.1 | 12.4 | 49.8 | 36.5 | 13.3 | yes | Greater than additive |
| 21 | 26.8 | 25.8 | 61.8 | 52.6 | 9.2 | yes | Greater than additive |
| 24 | 33.2 | 31.7 | 77.3 | 64.9 | 12.4 | yes | Greater than additive |
| 27 | 56.2 | 61.3 | 83.6 | 117.5 | −33.9 | yes | Less than additive |
| 30 | 54.9 | 56.3 | 74.8 | 111.2 | −36.4 | yes | Less than additive |
| 38 | −25.1 | −16.9 | 30.5 | −42.0 | 72.5 | yes | Greater than additive |
| 41 | −32.5 | −21.6 | 22.1 | −54.1 | 76.2 | yes | Greater than additive |

Table 83 shows the % of tumor growth inhibition of PM00104 and bevacizumab administered as single agents and in combination at a dose of 0.9 mg/kg/day of PM00104 and 2.5 mg/kg/day of bevacizumab. Additionally, the potentiation and the degree of additivity of the combination of PM00104 with bevacizumab at said doses are provided.

TABLE 83

| | % Inhibition | | | Expected | Actual | | Degree of |
|---|---|---|---|---|---|---|---|
| Day | G2 | G4 | G8 | Response | Response | Potentiation | Response |
| 9 | 3.7 | 0.6 | −5.5 | 4.3 | −9.8 | no | — |
| 13 | 2.2 | 18.3 | 36.9 | 20.5 | 16.4 | yes | Greater than additive |
| 16 | 24.1 | 25.7 | 33.6 | 49.8 | −16.2 | yes | Less than additive |
| 21 | 26.8 | 40.3 | 49.5 | 67.1 | −17.6 | yes | Less than additive |
| 24 | 33.2 | 48.0 | 74.4 | 81.2 | −6.8 | yes | Less than additive |
| 27 | 56.2 | 73.1 | 81.5 | 129.3 | −47.8 | yes | Less than additive |
| 30 | 54.9 | 56.7 | 63.5 | 111.6 | −48.1 | yes | Less than additive |
| 38 | −25.1 | 3.2 | 16.6 | −21.9 | 38.5 | yes | Greater than additive |
| 41 | −32.5 | −4.7 | 8.6 | −37.2 | 45.8 | yes | Greater than additive |

Table 84 shows the % of tumor growth inhibition of PM00104 and temsirolimus administered as single agents and in combination at a dose of 0.9 mg/kg/day of PM00104 and 20 mg/kg/day of temsirolimus. Additionally, the potentiation and the degree of additivity of the combination of PM00104 with temsirolimus at said doses are provided.

TABLE 84

| | % Inhibition | | | Expected | Actual | | Degree of |
|---|---|---|---|---|---|---|---|
| Day | G2 | G5 | G9 | Response | Response | Potentiation | Response |
| 9 | 3.7 | −4.4 | −0.5 | −0.7 | 0.2 | no | — |
| 13 | 2.2 | 33.6 | 67.0 | 35.8 | 31.2 | yes | Greater than additive |
| 16 | 24.1 | 31.6 | 71.8 | 55.7 | 16.1 | yes | Greater than additive |
| 21 | 26.8 | 53.2 | 82.0 | 80.0 | 2.0 | yes | Additive |
| 24 | 33.2 | 64.1 | 94.7 | 97.3 | −2.6 | yes | Less than additive |
| 27 | 56.2 | 84.1 | 96.6 | 140.3 | −43.7 | yes | Less than additive |
| 30 | 54.9 | 76.1 | 91.2 | 131.0 | −39.8 | yes | Less than additive |
| 38 | −25.1 | 38.8 | 66.0 | 13.7 | 52.3 | yes | Greater than additive |
| 41 | −32.5 | 34.8 | 52.1 | 2.3 | 49.8 | yes | Greater than additive |

Table 85 shows the % of tumor growth inhibition of PM00104 and temsirolimus administered as single agents and in combination at a dose of 0.9 mg/kg/day of PM00104 and 10 mg/kg/day of temsirolimus. Additionally, the potentiation and the degree of additivity of the combination of PM00104 with temsirolimus at said doses are provided.

TABLE 85

| | % Inhibition | | | Expected | Actual | | Degree of |
|---|---|---|---|---|---|---|---|
| Day | G2 | G6 | G10 | Response | Response | Potentiation | Response |
| 9 | 3.7 | −1.9 | −3.0 | 1.8 | −4.8 | no | — |
| 13 | 2.2 | 39.6 | 88.3 | 41.8 | 46.5 | yes | Greater than additive |
| 16 | 24.1 | 44.5 | 70.8 | 68.6 | 2.2 | yes | Additive |
| 21 | 26.8 | 59.5 | 77.7 | 86.3 | −8.6 | yes | Less than additive |
| 24 | 33.2 | 81.1 | 91.7 | 114.3 | −22.6 | yes | Less than additive |
| 27 | 56.2 | 87.1 | 95.5 | 143.3 | −47.8 | yes | Less than additive |
| 30 | 54.9 | 78.0 | 88.8 | 132.9 | −44.1 | yes | Less than additive |
| 38 | −25.1 | 48.0 | 59.9 | 22.9 | 37.0 | yes | Greater than additive |
| 41 | −32.5 | 33.6 | 58.3 | 1.1 | 57.2 | yes | Greater than additive |

According to this assay it was found that the combination of PM00104 and bevacizumab resulted in potentiation of antitumor activity over results obtained with either of the single agent control groups. At both doses of bevacizumab this potentiation was graded as greater than additive at the end of the assay.

According to this assay it was found that the combination of PM00104 and temsirolimus resulted in potentiation of antitumor activity over results obtained with either of the single agent control groups. At both doses of temsirolimus this potentiation was graded as greater than additive at the end of the assay.

### Example 20

In vivo studies to determine the effect of PM00104 in combination with gemcitabine in human lung cancer xenografts.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation. The Vehicle Control group contained 15 mice and the treated groups had each 9 mice/group.

The tumor model used in this study was NCI-H460 cell line which is a human NSCLC cell line obtained from the ATCC (Manassas, Va.). This cell line was grown and implanted to the animals as described in Example 19. Cells from in vitro passage 9 were those implanted SC into study mice.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 8.

PM00104 was provided in the form of vials of lyophilized PM00104 powder which was reconstituted with water for injection. Gemcitabine was provided in the form of a solid white powder containing gemcitabine HCl, which was reconstituted in 0.9% saline.

Study groups and treatment regimens are listed in table 86.

TABLE 86

| Group | Dose | Route | Schedule | Test material |
|---|---|---|---|---|
| G1 | 10 ml/kg/day | IV | A | 0.18% Placebo |
| (Control group) | 10 ml/kg/day | IP | A | 0.9% Saline |
| G2 | 0.9 mg/kg/day | IV | A | PM00104 |
| G3 | 180 mg/kg/day | IP | A | Gemcitabine |
| G4 | 90 mg/kg/day | IP | A | Gemcitabine |
| G5 | 0.9 mg/kg/day | IV | A | PM00104 |
| | 180 mg/kg/day | IP | A | Gemcitabine |
| G6 | 0.9 mg/kg/day | IV | A | PM00104 |
| | 90 mg/kg/day | IP | A | Gemcitabine |

A: DPI 8, 15, and 22;
Placebo: 500 mg Sucrose + 34 mg Potassium Phosphate + Phosphoric acid q.s. pH 3.8-4.4

Tumor size measurements were recorded 2-3 times/week from the treatment initiation until the termination of the study. Tumor growth inhibition was assessed comparing the mean tumor weight between the two agents in combination (PM00104 and gemcitabine) against gemcitabine mean tumor weight, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

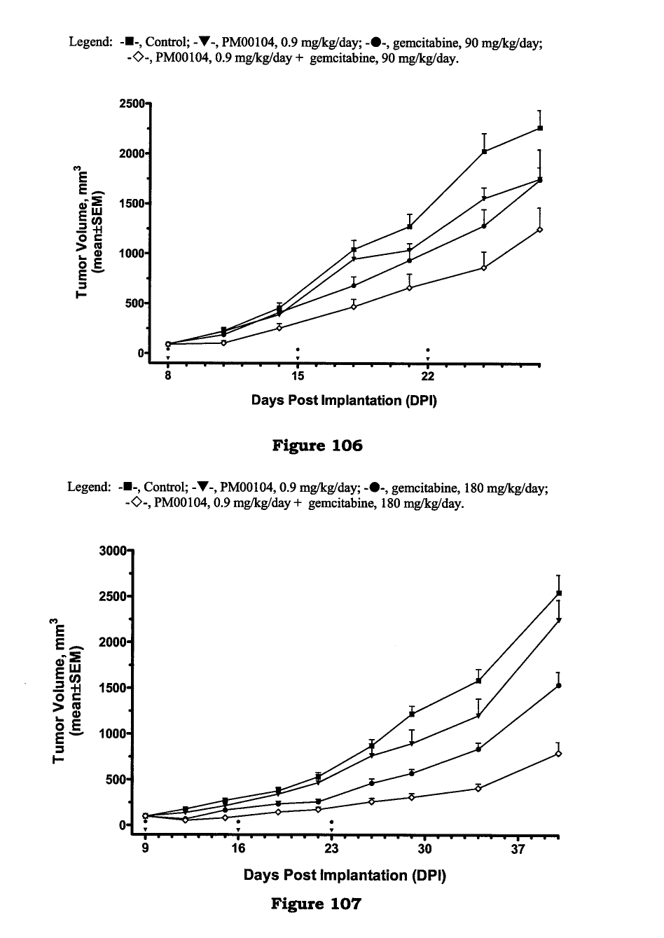

Table 87 reports the % T/C values obtained with each of the treatments and FIGS. 105-106 show the tumor volume evaluation (mean±SEM) of NCI-H460 tumors in mice treated with control, PM00104, gemcitabine and the corresponding combinations.

TABLE 87

| | % T/C on day | | | | | | |
|---|---|---|---|---|---|---|---|
| Group | 8 | 11 | 14 | 18 | 21 | 25 | 28 |
| G1 | — | — | — | — | — | — | — |
| G2 | 103.0 | 98.5 | 85.6 | 90.8 | 81.2 | 76.7 | 77.3 |
| G3 | 101.3 | 47.4 | 67.5 | 52.0 | 69.3 | 58.7 | 56.8 |
| G4 | 108.6 | 83.2 | 90.2 | 65.3 | 73.4 | 63.2 | 76.9 |
| G5 | 100.8 | 35.5 | 78.3 | 54.3 | 56.8 | 43.2 | 49.4 |
| G6 | 100.1 | 45.9 | 55.5 | 45.0 | 51.8 | 42.6 | 55.2 |

Table 88 shows the % of tumor growth inhibition of PM00104 and gemcitabine administered as single agents and in combination at a dose of 0.9 mg/kg/day of PM00104 and 180 mg/kg/day of gemcitabine. Additionally, the potentiation and the degree of additivity of the combination of PM00104 with gemcitabine at said doses are provided.

TABLE 88

| | % Inhibition | | | Expected | Actual | | Degree of |
|---|---|---|---|---|---|---|---|
| Day | G2 | G3 | G5 | Response | Response | Potentiation | Response |
| 8 | −3.0 | −1.3 | −0.8 | −4.3 | 3.5 | no | — |
| 11 | 1.5 | 52.6 | 64.5 | 54.1 | 10.4 | yes | Greater than additive |
| 14 | 14.4 | 32.5 | 21.7 | 46.9 | −25.2 | no | — |
| 18 | 9.2 | 48.0 | 45.7 | 57.2 | −11.5 | yes | Less than additive |
| 21 | 18.8 | 30.7 | 43.2 | 49.5 | −6.3 | yes | Less than additive |
| 25 | 23.3 | 41.3 | 56.8 | 64.6 | −7.8 | yes | Less than additive |
| 28 | 22.7 | 43.2 | 50.6 | 65.9 | −15.3 | yes | Less than additive |

Table 89 shows the % of tumor growth inhibition of PM00104 and gemcitabine administered as single agents and in combination at a dose of 0.9 mg/kg/day of PM00104 and 90 mg/kg/day of gemcitabine. Additionally, the potentiation and the degree of additivity of the combination of PM00104 with gemcitabine at said doses are provided.

TABLE 89

| | % Inhibition | | | Expected | Actual | | Degree of |
|---|---|---|---|---|---|---|---|
| Day | G2 | G4 | G6 | Response | Response | Potentiation | Response |
| 8 | −3.0 | −8.6 | −0.1 | −11.6 | 11.5 | no | — |
| 11 | 1.5 | 16.8 | 54.1 | 18.3 | 35.8 | yes | Greater than additive |
| 14 | 14.4 | 9.8 | 44.5 | 24.2 | 20.3 | yes | Greater than additive |
| 18 | 9.2 | 34.7 | 55.0 | 43.9 | 11.1 | yes | Greater than additive |
| 21 | 18.8 | 26.6 | 48.2 | 45.4 | 2.8 | yes | Additive |
| 25 | 23.3 | 36.8 | 57.4 | 60.1 | −2.7 | yes | Additive |
| 28 | 22.7 | 23.1 | 44.8 | 45.8 | −1.0 | yes | Additive |

According to this assay it was found that the combination of PM00104 and gemcitabine resulted in potentiation of antitumor activity. The potentiation of the combination having a lower dose of gemcitabine was graded as additive at the end of the study.

### Example 21

In vivo studies to determine the effect of PM00104 in combination with gemcitabine in human lung cancer xenografts.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation. The Vehicle Control group contained 13 mice and the treated groups had each 9 mice/group.

The tumor model used in this study was CaLu-6 cell line which is a human lung cancer cell line obtained from the ATCC (Manassas, Va.). CaLu-6 cells were grown in Eagle's Minimum Essential Medium (MEM), 10% FBS and 2 mM L-glutamine. Cells from in vitro passage 12 were implanted SC into study mice using a 1 ml syringe with a 13G trocar: 5×10⁶ cells/mouse in 0.2 ml 50% Matrigel/50% MEM medium of CaLu-6 without serum or antibiotics. Bacterial culture was taken on cells used to implant the study. All cultures were negative for bacterial contamination at both 24 and 48 hours post-implant.

Tumor measurements were determined as disclosed in Example 4. When tumors reached an appropriated volume, within the size range of 99±17 mm³ (mean±SD), mice were randomized into the treatment and control groups based on tumor weight by using LabCat® In Life module V 8.0 SP1 tumor tracking and measurement software. Treatments were initiated on DPI 9.

PM00104 was provided in the form of vials of lyophilized PM00104 powder which was reconstituted with water for injection. Gemcitabine was provided in the form of a solid white powder containing gemcitabine HCl, which was reconstituted in 0.9% saline.

Study groups and treatment regimens are listed in table 90.

TABLE 90

| Group | Dose | Route | Schedule | Test material |
|---|---|---|---|---|
| G1 | 10 ml/kg/day | IV | A | 0.18% Placebo |
| (Control group) | 10 ml/kg/day | IP | A | 0.9% Saline |
| G2 | 0.9 mg/kg/day | IV | A | PM00104 |
| G3 | 180 mg/kg/day | IP | A | Gemcitabine |
| G4 | 90 mg/kg/day | IP | A | Gemcitabine |
| G5 | 0.9 mg/kg/day | IV | A | PM00104 |
| | 180 mg/kg/day | IP | A | Gemcitabine |
| G6 | 0.9 mg/kg/day | IV | A | PM00104 |
| | 90 mg/kg/day | IP | A | Gemcitabine |

A: DPI 9, 16, and 23;
Placebo: 500 mg Sucrose + 34 mg Potassium Phosphate + Phosphoric acid q.s. pH 3.8-4.4

Tumor size measurements were recorded 2-3 times/week from the treatment initiation until the termination of the study. Tumor growth inhibition was assessed comparing the mean tumor weight between the two agents in combination (PM00104 and gemcitabine) against gemcitabine mean tumor weight, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

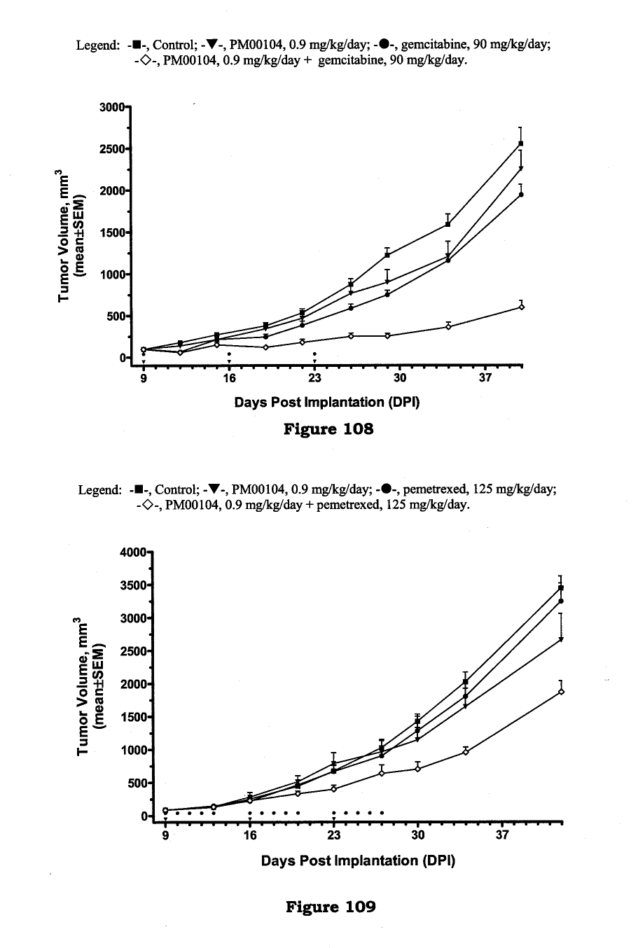

Table 91 reports the % T/C values obtained with each of the treatments and FIGS. 107-108 show the tumor volume evaluation (mean±SEM) of CaLu-6 tumors in mice treated with control, PM00104, gemcitabine and the corresponding combinations.

TABLE 91

| | % T/C on day | | | | | | | | |
|---|---|---|---|---|---|---|---|---|---|
| Group | 9 | 12 | 15 | 19 | 22 | 26 | 29 | 34 | 40 |
| G1 | — | — | — | — | — | — | — | — | — |
| G2 | 102.6 | 77.9 | 78.8 | 90.6 | 87.8 | 87.6 | 73.5 | 75.8 | 88.3 |
| G3 | 99.6 | 39.0 | 61.0 | 62.5 | 48.7 | 53.0 | 46.8 | 52.8 | 60.4 |
| G4 | 100.6 | 37.8 | 78.8 | 64.8 | 72.0 | 67.1 | 61.3 | 72.8 | 76.1 |
| G5 | 100.9 | 30.7 | 30.4 | 39.0 | 32.8 | 29.8 | 25.4 | 25.8 | 31.2 |
| G6 | 98.5 | 31.8 | 55.0 | 31.3 | 33.4 | 28.5 | 20.7 | 22.6 | 23.3 |

Table 92 shows the % of tumor growth inhibition of PM00104 and gemcitabine administered as single agents and in combination at a dose of 0.9 mg/kg/day of PM00104 and 180 mg/kg/day of gemcitabine. Additionally, the potentiation and the degree of additivity of the combination of PM00104 with gemcitabine at said doses are provided.

TABLE 92

| | % Inhibition | | | Expected | Actual | | Degree of |
|---|---|---|---|---|---|---|---|
| Day | G2 | G3 | G5 | Response | Response | Potentiation | Response |
| 9 | −2.6 | 0.4 | −0.9 | −2.2 | 1.3 | no | — |
| 12 | 22.1 | 61.0 | 69.3 | 83.1 | −13.8 | yes | Less than additive |
| 15 | 21.2 | 39.0 | 69.6 | 60.2 | 9.4 | yes | Greater than additive |
| 19 | 9.4 | 37.5 | 61.0 | 46.9 | 14.1 | yes | Greater than additive |
| 22 | 12.2 | 51.3 | 67.2 | 63.5 | 3.7 | yes | Additive |
| 26 | 12.4 | 47.0 | 70.2 | 59.4 | 10.8 | yes | Greater than additive |
| 29 | 26.5 | 53.2 | 74.6 | 79.7 | −5.1 | yes | Additive |
| 34 | 24.2 | 47.2 | 74.2 | 71.4 | 2.8 | yes | Additive |
| 40 | 11.7 | 39.6 | 68.8 | 51.3 | 17.5 | yes | Greater than additive |

Table 93 shows the % of tumor growth inhibition of PM00104 and gemcitabine administered as single agents and in combination at a dose of 0.9 mg/kg/day of PM00104 and 90 mg/kg/day of gemcitabine. Additionally, the potentiation and the degree of additivity of the combination of PM00104 with gemcitabine at said doses are provided.

TABLE 93

| | % Inhibition | | | Expected | Actual | | Degree of |
|---|---|---|---|---|---|---|---|
| Day | G2 | G4 | G6 | Response | Response | Potentiation | Response |
| 9 | −2.6 | −0.6 | 1.5 | −3.2 | 4.7 | no | — |
| 12 | 22.1 | 62.2 | 68.2 | 84.3 | −16.1 | yes | Less than additive |
| 15 | 21.2 | 21.2 | 45.0 | 42.4 | 2.6 | yes | Additive |
| 19 | 9.4 | 35.2 | 68.7 | 44.6 | 24.1 | yes | Greater than additive |
| 22 | 12.2 | 28.0 | 66.6 | 40.2 | 26.4 | yes | Greater than additive |
| 26 | 12.4 | 32.9 | 71.5 | 45.3 | 26.2 | yes | Greater than additive |
| 29 | 26.5 | 38.7 | 79.3 | 65.2 | 14.1 | yes | Greater than additive |
| 34 | 24.2 | 27.2 | 77.4 | 51.4 | 26.0 | yes | Greater than additive |
| 40 | 11.7 | 23.9 | 76.7 | 35.6 | 41.1 | yes | Greater than additive |

According to this assay it was found that the combination of PM00104 and gemcitabine resulted in potentiation of antitumor activity. At both doses of gemcitabine this potentiation was graded as greater than additive at the end of the assay.

### Example 22

In vivo studies to determine the effect of PM00104 in combination with pemetrexed in human lung cancer xenografts.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation. The Vehicle Control group contained 15 mice and the treated groups had each 10 mice/group.

The tumor model used in this study was CaLu-6 cell line which is a human lung cell line obtained from the ATCC (Manassas, Va.). This cell line was grown and implanted to the animals as described in Example 21. Cells from in vitro passage 10 were those implanted SC into study mice.

Tumor measurements were determined as disclosed in Example 4. When tumors reached an appropriated volume, within the size range of 86±16 mm³ (mean±SD), mice were randomized into the treatment and control groups based on tumor weight by using LabCat® In Life module V 8.0 SP1 tumor tracking and measurement software. Treatments were initiated on DPI 9.

PM00104 was provided in the form of vials of lyophilized PM00104 powder which was reconstituted with water for injection. Pemetrexed was provided in the form of a solid powder containing pemetrexed disodium, which was reconstituted in 0.9% saline.

Study groups and treatment regimens are listed in table 94.

TABLE 94

| Group | Dose | Route | Schedule | Test material |
|---|---|---|---|---|
| G1 | 10 ml/kg/day | IV | A | 0.18% Placebo |
| (Control group) | 10 ml/kg/day | IP | B | 0.9% Saline |
| G2 | 0.9 mg/kg/day | IV | A | PM00104 |
| G3 | 125 mg/kg/day | IP | B | Pemetrexed |
| G4 | 100 mg/kg/day | IP | B | Pemetrexed |
| G5 | 0.9 mg/kg/day | IV | A | PM00104 |
| | 125 mg/kg/day | IP | B | Pemetrexed |
| G6 | 0.9 mg/kg/day | IV | A | PM00104 |
| | 100 mg/kg/day | IP | B | Pemetrexed |

A: DPI 9, 16, and 23;
B: DPI 9-13, 16-20, and 23-27
Placebo: 500 mg Sucrose + 34 mg Potassium Phosphate + Phosphoric acid q.s. pH 3.8-4.4

Tumor size measurements were recorded 2-3 times/week from the treatment initiation until the termination of the study. Tumor growth inhibition was assessed comparing the mean tumor weight between the two agents in combination (PM00104 and pemetrexed) against pemetrexed mean tumor weight, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

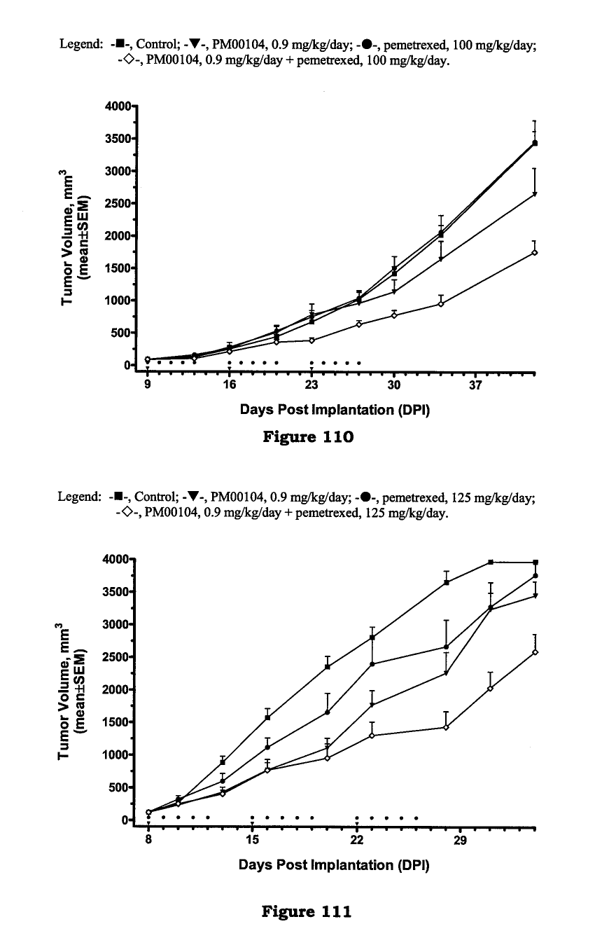

Table 95 reports the % T/C values obtained with each of the treatments and FIGS. 109-110 show the tumor volume evaluation (mean±SEM) of CaLu-6 tumors in mice treated with control, PM00104, pemetrexed and the corresponding combinations.

TABLE 95

| | % T/C on day | | | | | | | | |
|---|---|---|---|---|---|---|---|---|---|
| Group | 9 | 13 | 16 | 20 | 23 | 27 | 30 | 34 | 42 |
| G1 | — | — | — | — | — | — | — | — | |
| G2 | 104.1 | 106.0 | 111.4 | 115.4 | 116.8 | 93.9 | 80.3 | 81.5 | 77.2 |
| G3 | 99.6 | 116.6 | 86.6 | 104.8 | 98.7 | 87.9 | 90.0 | 89.1 | 94.2 |
| G4 | 103.4 | 126.5 | 103.5 | 120.6 | 111.5 | 101.8 | 105.8 | 102.2 | 100.5 |
| G5 | 99.7 | 103.1 | 89.0 | 74.9 | 59.2 | 62.0 | 49.3 | 47.2 | 54.2 |
| G6 | 102.5 | 82.3 | 84.1 | 81.2 | 57.3 | 62.0 | 54.6 | 47.5 | 51.0 |

Table 96 shows the % of tumor growth inhibition of PM00104 and pemetrexed administered as single agents and in combination at a dose of 0.9 mg/kg/day of PM00104 and 125 mg/kg/day of pemetrexed. Additionally, the potentiation and the degree of additivity of the combination of PM00104 with pemetrexed at said doses are provided.

TABLE 96

| | % Inhibition | | | Expected | Actual | | Degree of |
|---|---|---|---|---|---|---|---|
| Day | G2 | G3 | G5 | Response | Response | Potentiation | Response |
| 9 | −4.1 | 0.4 | 0.3 | −3.7 | 4.0 | no | — |
| 13 | −6.0 | −16.6 | −3.1 | −22.6 | 19.5 | no | — |
| 16 | −11.4 | 13.4 | 11.0 | 2.0 | 9.0 | no | — |
| 20 | −15.4 | −4.8 | 25.1 | −20.2 | 45.3 | yes | Greater than additive |
| 23 | −16.8 | 1.3 | 40.8 | −15.5 | 56.3 | yes | Greater than additive |
| 27 | 6.1 | 12.1 | 38.0 | 18.2 | 19.8 | yes | Greater than additive |
| 30 | 19.7 | 10.0 | 50.7 | 29.7 | 21.0 | yes | Greater than additive |
| 34 | 18.5 | 10.9 | 52.8 | 29.4 | 23.4 | yes | Greater than additive |
| 42 | 22.8 | 5.8 | 45.8 | 28.6 | 17.2 | yes | Greater than additive |

Table 97 shows the % of tumor growth inhibition of PM00104 and pemetrexed administered as single agents and in combination at a dose of 0.9 mg/kg/day of PM00104 and 100 mg/kg/day of pemetrexed. Additionally, the potentiation and the degree of additivity of the combination of PM00104 with pemetrexed at said doses are provided.

TABLE 97

| | % Inhibition | | | Expected | Actual | | Degree of |
|---|---|---|---|---|---|---|---|
| Day | G2 | G4 | G6 | Response | Response | Potentiation | Response |
| 9 | −4.1 | −3.4 | −2.5 | −7.5 | 5.0 | no | — |
| 13 | −6.0 | −26.5 | 17.7 | −32.5 | 50.2 | yes | Greater than additive |
| 16 | −11.4 | −3.5 | 15.9 | −14.9 | 30.8 | yes | Greater than additive |
| 20 | −15.4 | −20.6 | 18.8 | −36.0 | 54.8 | yes | Greater than additive |
| 23 | −16.8 | −11.5 | 42.7 | −28.3 | 71.0 | yes | Greater than additive |
| 27 | 6.1 | −1.8 | 38.0 | 4.3 | 33.7 | yes | Greater than additive |
| 30 | 19.7 | −5.8 | 45.4 | 13.9 | 31.5 | yes | Greater than additive |
| 34 | 18.5 | −2.2 | 52.5 | 16.3 | 36.2 | yes | Greater than additive |
| 42 | 22.8 | −0.5 | 49.0 | 22.3 | 26.7 | yes | Greater than additive |

According to this assay it was found that the combination of PM00104 and pemetrexed resulted in potentiation of antitumor activity. At both doses of pemetrexed dose this potentiation was graded as greater than additive at the end of the assay.

### Example 23

In vivo studies to determine the effect of PM00104 in combination with pemetrexed in human lung cancer xenografts.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation. The Vehicle Control group contained 14 mice and the treated groups had each 9 mice/group.

The tumor model used in this study was NCI-H460 cell line which is a human lung cell line obtained from the ATCC (Manassas, Va.). This cell line was grown and implanted to the animals as described in Example 19. Cells from in vitro passage 16 were those implanted SC into study mice.

Tumor measurements were determined as disclosed in Example 4. When tumors reached an appropriated volume, within the size range of 118±32 mm³ (mean±SD), mice were randomized into the treatment and control groups based on tumor weight by using LabCat® In Life module V 8.0 SP1 tumor tracking and measurement software. Treatments were initiated on DPI 8.

PM00104 was provided in the form of vials of lyophilized PM00104 powder which was reconstituted with water for injection. Pemetrexed was provided in the form of a solid powder containing pemetrexed disodium, which was reconstituted in 0.9% saline.

Study groups and treatment regimens are listed in table 98.

TABLE 98

| Group | Dose | Route | Schedule | Test material |
|---|---|---|---|---|
| G1 | 10 ml/kg/day | IV | A | 0.18% Placebo |
| (Control group) | 10 ml/kg/day | IP | B | 0.9% Saline |
| G2 | 0.9 mg/kg/day | IV | A | PM00104 |
| G3 | 125 mg/kg/day | IP | B | Pemetrexed |
| G4 | 100 mg/kg/day | IP | B | Pemetrexed |
| G5 | 0.9 mg/kg/day | IV | A | PM00104 |
| | 125 mg/kg/day | IP | B | Pemetrexed |
| G6 | 0.9 mg/kg/day | IV | A | PM00104 |
| | 100 mg/kg/day | IP | B | Pemetrexed |

A: DPI 8, 15, and 22;
B: DPI 8-12, 15-19, and 22-26
Placebo: 500 mg Sucrose + 34 mg Potassium Phosphate + Phosphoric acid q.s. pH 3.8-4.4

Tumor size measurements were recorded 2-3 times/week from the treatment initiation until the termination of the study. Tumor growth inhibition was assessed comparing the mean tumor weight between the two agents in combination (PM00104 and pemetrexed) against pemetrexed mean tumor weight, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

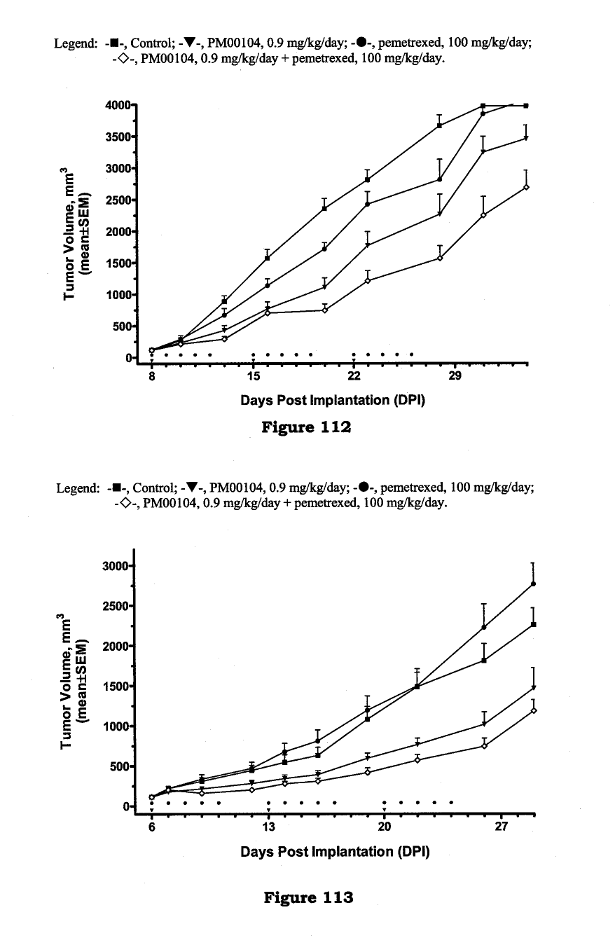

Table 99 reports the % T/C values obtained with each of the treatments and FIGS. 111-112 show the tumor volume evaluation (mean±SEM) of NCI-H460 tumors in mice treated with control, PM00104, pemetrexed and the corresponding combinations.

TABLE 99

| | % T/C on day | | | | | | | |
|---|---|---|---|---|---|---|---|---|
| Group | 8 | 10 | 13 | 16 | 20 | 23 | 28 | 31 |
| G1 | — | — | — | — | — | — | — | — |
| G2 | 101.5 | 86.5 | 48.5 | 49.0 | 47.2 | 63.0 | 61.9 | 81.7 |
| G3 | 102.6 | 119.0 | 67.5 | 71.0 | 70.4 | 85.4 | 72.9 | 82.6 |
| G4 | 103.0 | 107.3 | 75.7 | 72.4 | 72.9 | 86.3 | 76.8 | 96.9 |
| G5 | 99.5 | 92.7 | 45.4 | 48.7 | 40.5 | 46.4 | 39.2 | 51.2 |
| G6 | 97.9 | 78.3 | 32.9 | 44.7 | 31.6 | 43.1 | 42.8 | 56.6 |

Table 100 shows the % of tumor growth inhibition of PM00104 and pemetrexed administered as single agents and in combination at a dose of 0.9 mg/kg/day of PM00104 and 125 mg/kg/day of pemetrexed. Additionally, the potentiation and the degree of additivity of the combination of PM00104 with pemetrexed at said doses are provided.

TABLE 100

| | % Inhibition | | | Expected | Actual | | Degree of |
|---|---|---|---|---|---|---|---|
| Day | G2 | G3 | G5 | Response | Response | Potentiation | Response |
| 8 | −1.5 | −2.6 | 0.5 | −4.1 | 4.6 | no | — |
| 10 | 13.5 | −19.0 | 7.3 | −5.5 | 12.8 | no | — |
| 13 | 51.5 | 32.5 | 54.6 | 84.0 | −29.4 | no | — |
| 16 | 51.0 | 29.0 | 51.3 | 80.0 | −28.7 | no | — |
| 20 | 52.8 | 29.6 | 59.5 | 82.4 | −22.9 | yes | Less than additive |
| 23 | 37.0 | 14.6 | 53.6 | 51.6 | 2.0 | yes | Additive |
| 28 | 38.1 | 27.1 | 60.8 | 65.2 | −4.4 | yes | Additive |
| 31 | 18.3 | 17.4 | 48.8 | 35.7 | 13.1 | yes | Greater than additive |

Table 101 shows the % of tumor growth inhibition of PM00104 and pemetrexed administered as single agents and in combination at a dose of 0.9 mg/kg/day of PM00104 and 100 mg/kg/day of pemetrexed. Additionally, the potentiation and the degree of additivity of the combination of PM00104 with pemetrexed at said doses are provided.

TABLE 101

| | % Inhibition | | | Expected | Actual | | Degree of |
|---|---|---|---|---|---|---|---|
| Day | G2 | G4 | G6 | Response | Response | Potentiation | Response |
| 8 | −1.5 | −3.0 | 2.1 | −4.5 | 6.6 | yes | Additive |
| 10 | 13.5 | −7.3 | 21.7 | 6.2 | 15.5 | yes | Greater than additive |
| 13 | 51.5 | 24.3 | 67.1 | 75.8 | −8.7 | yes | Less than additive |
| 16 | 51.0 | 27.6 | 55.3 | 78.6 | −23.3 | yes | Less than additive |
| 20 | 52.8 | 27.1 | 68.4 | 79.9 | −11.5 | yes | Less than additive |
| 23 | 37.0 | 13.7 | 56.9 | 50.7 | 6.2 | yes | Additive |
| 28 | 38.1 | 23.2 | 57.2 | 61.3 | −4.1 | yes | Additive |
| 31 | 18.3 | 3.1 | 43.4 | 21.4 | 22.0 | yes | Greater than additive |

According to this assay it was found that the combination of PM00104 and pemetrexed resulted in potentiation of antitumor activity. At both doses of pemetrexed this potentiation was graded as greater than additive at the end of the assay.

### Example 24

In vivo studies to determine the effect of PM00104 in combination with pemetrexed in human mesothelioma xenografts.

Female athymic nude mice (Harlan Sprague Dawley, Madison, Wis.) were utilized for all experiments. Animals were housed in ventilated rack caging with food and water ad libitum. The mice were acclimated for at least 5 days prior to tumor implantation. The Vehicle Control group contained 15 mice and the treated groups had each 10 mice/group.

The tumor model used in this study was H-Meso-1 cell line which is a human mesothelioma cell line obtained from the DTP, DCTD tumor repository. H-Meso-1 cells were grown in RPMI-1640 medium, 10% FBS, and 2 mM L-glutamine. Cells were implanted SC into study mice using a 1 ml syringe with a 13G trocar: 5×10⁶ cells/mouse in 0.2 ml 50% Matrigel/50% RPMI medium of H-Meso-1 without serum or antibiotics. Bacterial culture was taken on cells used to implant the study. All cultures were negative for bacterial contamination at both 24 and 48 hours post-implant.

Tumor measurements were determined as disclosed in Example 4. When tumors reached an appropriated volume, within the size range of 175±100 mm³ (mean±SD), mice were randomized into the treatment and control groups based on tumor weight by using LabCat® In Life module V 8.0 SP1 tumor tracking and measurement software. Treatments were initiated on DPI 6.

PM00104 was provided in the form of vials of lyophilized PM00104 powder which was reconstituted with water for injection. Pemetrexed was provided in the form of a solid powder containing pemetrexed disodium, which was reconstituted in 0.9% saline.

Study groups and treatment regimens are listed in table 102.

TABLE 102

| Group | Dose | Route | Schedule | Test material |
|---|---|---|---|---|
| G1 | 10 ml/kg/day | IV | A | 0.18% Placebo |
| (Control group) | 10 ml/kg/day | IP | B | 0.9% Saline |
| G2 | 0.9 mg/kg/day | IV | A | PM00104 |
| G3 | 0.45 mg/kg/day | IV | A | PM00104 |
| G4 | 100 mg/kg/day | IP | B | Pemetrexed |
| G5 | 0.9 mg/kg/day | IV | A | PM00104 |
| | 100 mg/kg/day | IP | B | Pemetrexed |
| G6 | 0.45 mg/kg/day | IV | A | PM00104 |
| | 100 mg/kg/day | IP | B | Pemetrexed |

A: DPI 6, 13, and 20;
B: DPI 6-10, 13-17, and 20-24
Placebo: 500 mg Sucrose + 34 mg Potassium Phosphate + Phosphoric acid q.s. pH 3.8-4.4

Tumor size measurements were recorded 2-3 times/week from the treatment initiation until the termination of the study. Tumor growth inhibition was assessed comparing the mean tumor weight between the two agents in combination (PM00104 and pemetrexed) against pemetrexed mean tumor weight, at the different concentrations assayed.

Mean, standard deviation and standard error of the mean were determined for tumor volume for all animal groups at all assessments. Student's t test was performed on tumor volumes at each measurement day, including at the end of the study, to determine whether there were any statistically significant differences between combination treatment groups and single monotherapy treatment groups.

The definition and criteria for the evaluation of potentiation and the degree of additivity for the combination therapy were the same as those disclose in Example 4.

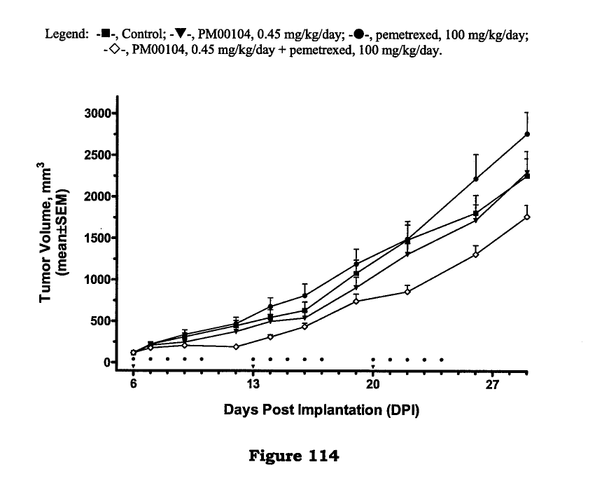

Table 103 reports the % T/C values obtained with each of the treatments and FIGS. 113-114 show the tumor volume evaluation (mean±SEM) of H-Meso-1 tumors in mice treated with control, PM00104, pemetrexed and the corresponding combinations.

TABLE 103

| | % T/C on day | | | | | | | | | |
|---|---|---|---|---|---|---|---|---|---|---|
| Group | 6 | 7 | 9 | 12 | 14 | 16 | 19 | 22 | 26 | 29 |
| G1 | — | — | — | — | — | — | — | — | — | — |
| G2 | 98.5 | 80.3 | 69.7 | 63.3 | 62.9 | 62.1 | 55.1 | 51.6 | 56.0 | 64.9 |
| G3 | 100.4 | 94.8 | 79.5 | 84.0 | 91.3 | 85.6 | 84.0 | 88.4 | 95.1 | 101.9 |
| G4 | 102.1 | 100.2 | 109.0 | 105.7 | 124.2 | 128.6 | 110.4 | 100.8 | 122.9 | 122.4 |
| G5 | 99.0 | 91.8 | 51.9 | 45.4 | 51.1 | 49.0 | 38.5 | 38.3 | 41.0 | 52.4 |
| G6 | 101.0 | 79.4 | 66.3 | 42.7 | 56.5 | 68.8 | 68.8 | 57.9 | 72.4 | 78.2 |

Table 104 shows the % of tumor growth inhibition of PM00104 and pemetrexed administered as single agents and in combination at a dose of 0.9 mg/kg/day of PM00104 and 100 mg/kg/day of pemetrexed. Additionally, the potentiation and the degree of additivity of the combination of PM00104 with pemetrexed at said doses are provided.

TABLE 104

| | % Inhibition | | | Expected | Actual | | Degree of |
|---|---|---|---|---|---|---|---|
| Day | G2 | G4 | G5 | Response | Response | Potentiation | Response |
| 6 | 1.5 | −2.1 | 1.0 | −0.6 | 1.6 | no | — |
| 7 | 19.7 | −0.2 | 8.2 | 19.5 | −11.3 | no | — |
| 9 | 30.3 | −9.0 | 48.1 | 21.3 | 26.8 | yes | Greater than additive |
| 12 | 36.7 | −5.7 | 54.6 | 31.0 | 23.6 | yes | Greater than additive |
| 14 | 37.1 | −24.2 | 48.9 | 12.9 | 36.0 | yes | Greater than additive |
| 16 | 37.9 | −28.6 | 51.0 | 9.3 | 41.7 | yes | Greater than additive |
| 19 | 44.9 | −10.4 | 61.5 | 34.5 | 27.0 | yes | Greater than additive |
| 22 | 48.4 | −0.8 | 61.7 | 47.6 | 14.1 | yes | Greater than additive |
| 26 | 44.0 | −22.9 | 59.0 | 21.1 | 37.9 | yes | Greater than additive |
| 29 | 35.0 | −22.4 | 47.6 | 12.6 | 35.0 | yes | Greater than additive |

Table 105 shows the % of tumor growth inhibition of PM00104 and pemetrexed administered as single agents and in combination at a dose of 0.45 mg/kg/day of PM00104 and 100 mg/kg/day of pemetrexed. Additionally, the potentiation and the degree of additivity of the combination of PM00104 with pemetrexed at said doses are provided.

TABLE 105

| | % Inhibition | | | Expected | Actual | | Degree of |
|---|---|---|---|---|---|---|---|
| Day | G3 | G4 | G6 | Response | Response | Potentiation | Response |
| 6 | −0.4 | −2.1 | −1.0 | −2.5 | 1.5 | no | — |
| 7 | 5.2 | −0.2 | 20.6 | 5.0 | 15.6 | yes | Greater than additive |
| 9 | 20.5 | −9.0 | 33.7 | 11.5 | 22.2 | yes | Greater than additive |
| 12 | 16.0 | −5.7 | 57.3 | 10.3 | 47.0 | yes | Greater than additive |
| 14 | 8.7 | −24.2 | 43.5 | −15.5 | 59.0 | yes | Greater than additive |
| 16 | 14.4 | −28.6 | 31.2 | −14.2 | 45.4 | yes | Greater than additive |
| 19 | 16.0 | −10.4 | 31.2 | 5.6 | 25.6 | yes | Greater than additive |
| 22 | 11.6 | −0.8 | 42.1 | 10.8 | 31.3 | yes | Greater than additive |
| 26 | 4.9 | −22.9 | 27.6 | −18.0 | 45.6 | yes | Greater than additive |
| 29 | −1.9 | −22.4 | 21.8 | −24.3 | 46.1 | yes | Greater than additive |

According to this assay it was found that the combination of PM00104 and pemetrexed resulted in potentiation of antitumor activity. At both doses of PM00104 this potentiation was graded as greater than additive at the end of the assay.

1. A method of treating cancer comprising administering to a patient in need of such treatment a therapeutically effective amount of PM00104, or a pharmaceutically acceptable salt thereof, and a therapeutically effective amount of another anticancer drug selected from tyrosine kinase inhibitors, mTOR inhibitors, antitumor platinum coordination complexes, antimetabolites, mitotic inhibitors, anthracyclines, topoisomerase I and/or II inhibitors, and antitumor monoclonal antibodies.

2. A method of potentiating the therapeutic efficacy of an anticancer drug selected from tyrosine kinase inhibitors, mTOR inhibitors, antitumor platinum coordination complexes, antimetabolites, mitotic inhibitors, anthracyclines; topoisomerase I and/or II inhibitors, and antitumor monoclonal antibodies in the treatment of cancer, which comprises administering to a patient in need thereof a therapeutically effective amount of PM00104, or a pharmaceutically acceptable salt thereof, in conjunction with said anticancer drug.

3-27. (canceled)

28. The method according to claim 1, wherein the cancer to be treated is selected from lung cancer, sarcoma, malignant melanoma, pleural mesothelioma, bladder carcinoma, prostate cancer, pancreas carcinoma, gastric carcinoma, ovarian cancer, hepatoma, breast cancer, colorectal cancer, kidney cancer, esophageal cancer, suprarenal cancer, parotid gland cancer, head and neck carcinoma, cervix cancer, mesothelioma, leukaemia, and lymphoma.

29. The method according to claim 28, wherein
PM00104 or a pharmaceutically acceptable salt thereof, and
the other anticancer drug selected from tyrosine kinase inhibitors, mTOR inhibitors, antitumor platinum coordination complexes, antimetabolites, mitotic inhibitors, anthracyclines, topoisomerase I and/or II inhibitors, and antitumor monoclonal antibodies,
form part of the same composition.

30. The method according to claim 28, wherein PM00104, or a pharmaceutically acceptable salt thereof, and the other anticancer drug selected from tyrosine kinase inhibitors, mTOR inhibitors, antitumor platinum coordination complexes, antimetabolites, mitotic inhibitors, anthracyclines, topoisomerase I and/or II inhibitors, and antitumor monoclonal antibodies, are provided as separate compositions for administration at the same time or at different times.

31. The method according to claim 30, wherein PM00104, or a pharmaceutically acceptable salt thereof, and the other anticancer drug selected from tyrosine kinase inhibitors, mTOR inhibitors, antitumor platinum coordination complexes, antimetabolites, mitotic inhibitors, anthracyclines, topoisomerase I and/or II inhibitors, and antitumor monoclonal antibodies, are provided as separate compositions for administration at different times.

32. A method according to claim 31, wherein the anticancer drug combined with PM00104 is a tyrosine kinase inhibitor.

33. A method according to claim 32, wherein the anticancer drug combined with PM00104 is a tyrosine kinase inhibitor selected from erlotinib sorafenib axitinib, bosutinib, cediranib, dasatinib, gefitinib, imatinib, canertinib, lapatinib, lestaurtinib, semaxanib, sunitinib, and vandetanib.

34. A method according to claim 31 wherein the anticancer drug combined with PM00104 is an mTOR inhibitor.

35. A method according to claim 34 wherein the anticancer drug combined with PM00104 is an mTOR inhibitor selected from temsirolimus, sirolimus, everolimus, and deforolimus.

36. A method according to claim 31 wherein the anticancer drug combined with PM00104 is an antitumor platinum coordination complex.

37. A method according to claim 36, wherein the anticancer drug combined with PM0104 is an antitumor platinum coordination complex selected from cisplatin, oxaliplatin, carboplatin, BBR3464, satraplatin, tetraplatin, ormiplatin, and iproplatin.

38. A method according to claim 31, wherein the anticancer drug combined with PM00104 is an antimetabolite.

39. A method according to claim 38, wherein the anticancer drug combined with PM00104 is an antimetabolite selected from 5-fluorouracil, gemcitabine, cytarabine, capecitabine, decitabine, floxuridine, 6-mercaptopurine, methotrexate, fludarabine, aminopterin, pemetrexed, raltitrexed, cladribine clofarabine, fludarabine, mercaptopurine, pentostatin, and thioguanine.

40. A method according to claim 31, wherein the anticancer drug combined with PM00104 is a mitotic inhibitor.

41. A method according to claim 40, wherein the anticancer drug combined with PM00104 is a mitotic inhibitor selected from paclitaxel, docetaxel, vinblastine, vincristine, vindesine, and vinorelbine.

42. A method according to claim 31, wherein the anticancer drug combined with PM00104 is an anthracycline.

43. A method according to claim 42, wherein the anticancer drug combined with PM00104 is an anthracycline selected from aunorubicin, doxorubicin, epirubicin, idarubicin, mitoxantrone, pixantrone, and valrubicin.

44. A method according to claim 31, wherein the anticancer drug combined with PM0104 is a topoisomerase I and/or II inhibitor.

45. A method according to claim 44, wherein the anticancer drug combined with PM00104 is a topoisomerase I and/or 11 inhibitor selected from topotecan, SN-38, irinotecan, camptothecine, rubitecan, etoposide, and teniposide.

46. A method according to claim 31, wherein the anticancer drug combined with PM00104 is an antitumor monoclonal antibody.

47. A method according to claim 46, Wherein the anticancer drug combined with PM00104 is an antitumor monoclonal antibody selected from bevacizumab, cetuximan, panitumumab, trastuzumab, rituximab, tositumomab, alemtuzumab, and gemtuzumab.

48. A kit for administering PM00104, or a pharmaceutically acceptable salt thereof, in combination with another anticancer drug selected from tyrosine kinase inhibitors, mTOR inhibitors, antitumor platinum coordination complexes, an metabolites, mitotic inhibitors, anthracyclines, topoisomerase I and/or II inhibitors, and antitumor monoclonal antibodies, comprising a dosage form of PM00104, or a pharmaceutically acceptable salt thereof, and/or a dosage form of another anticancer drug selected from tyrosine kinase inhibitors, mTOR inhibitors, antitumor platinum coordination complexes, antimetabolites, mitotic inhibitors, anthracyclines, topoisomerase I and/or II inhibitors, and antitumor monoclonal antibodies, and instructions for administering both drugs in combination.
